(19) United States
(12) Patent Application Publication
(10) Pub. No.: US 2022/0220211 A1
(43) Pub. Date: Jul. 14, 2022

(54) METHODS FOR TREATING PEANUT ALLERGY AND ENHANCING PEANUT ALLERGEN-SPECIFIC IMMUNOTHERAPY BY ADMINISTERING AN IL-4R ANTAGONIST

(71) Applicants: REGENERON PHARMACEUTICALS, INC., Tarrytown, NY (US); SANOFI BIOTECHNOLOGY, Paris (FR)

(72) Inventors: Jamie M. ORENGO, Cortlandt Manor, NY (US); Allen RADIN, New York, NY (US); Marcella RUDDY, Wellesley, MA (US); Heribert STAUDINGER, Green Brook, NJ (US); Qiufan WANG, New York, NY (US)

(21) Appl. No.: 17/570,547

(22) Filed: Jan. 7, 2022

Related U.S. Application Data
(60) Provisional application No. 63/135,238, filed on Jan. 8, 2021.

(30) Foreign Application Priority Data
Nov. 10, 2021 (EP) 21315241.6

(51) Int. Cl. C07K 16/28; A61P 37/08

(52) U.S. Cl. CPC C07K 16/2866; A61K 2039/545; A61P 37/08

(57) ABSTRACT

Methods for enhancing the efficacy, safety, and/or tolerability of a peanut allergen-specific immunotherapy regimen in a subject having a peanut allergy, comprising administering an interleukin-4 receptor (IL-4R) antagonist such as an anti-IL-4R antibody or antigen-binding fragment thereof in combination with the immunotherapy, are provided.

### CROSS-REFERENCE TO RELATED APPLICATIONS

This application claims priority to U.S. Provisional Patent Application No. 63/135,238, filed Jan. 8, 2021, and to European Patent Application No. 21315241.6, filed Nov. 10, 2021, the entire contents of each of which are incorporated by reference herein.

### SEQUENCE LISTING

The instant application contains a Sequence Listing which has been submitted electronically in ASCII format and is hereby incorporated by reference in its entirety. Said ASCII copy, created on Jan. 3, 2022, is named 40848_0110USU1_SL.txt and is 231,417 bytes in size.

### FIELD OF THE INVENTION

The present disclosure relates to the use of interleukin-4 receptor (IL-4R) antagonists to treat or reduce symptoms of peanut allergy and to improve the efficacy and/or tolerability of peanut allergen-specific immunotherapy regimens.

### BACKGROUND

Food allergy is a potentially life-threatening condition that affects up to 8% of young children and 3% to 5% of the entire United States population (Gupta et al., Pediatrics 2011, 128: e9-e17; Sicherer, JACI 2010, 125:1322-6). Unlike many other childhood allergies, peanut allergy typically persists into adulthood and is associated with a higher incidence of severe anaphylaxis as compared with other food allergies (Dyer, Allergy, Asthma, Proc 2015, 36:58-64). The current remedies for food allergy are food avoidance and treatment with medications such as injectable epinephrine for accidental exposures associated with severe allergic symptoms.

Although recent progress has been made in the treatment of food allergy through allergen-specific oral immunotherapy (OIT), there is an unmet need for a new therapy in food allergy. The aim of OIT is to induce desensitization and increase the threshold for allergen (e.g., peanut) ingestion and reduce the risks of allergic reactions after accidental ingestion. Like other forms of allergy immunotherapy, peanut OIT involves a slow up-dosing of exposure to allergen (peanut allergen) over time to desensitize or increase the threshold of reactivity to peanut. Once reaching a target level of peanut protein, subjects are continued on a maintenance dose of peanut protein to maintain desensitization.

Although many subjects on a maintenance dose of peanut protein have demonstrated desensitization to peanut (i.e., the ability to tolerate a level of exposure to peanut without an allergic reaction), up to 80% of subjects exhibit related adverse events (AEs) during OIT, with 42% experiencing systemic reactions and 49% experiencing gastrointestinal (GI) symptoms; although the majority of these reactions and symptoms are mild and decline with prolonged treatment, up to 20% of subjects are unable to complete the up-dosing regimen due to side effects (Virkud et al., JACI 2016, 1-7e). An additional issue with current OIT is its limited ability to induce clinical tolerance when subjects are taken off daily peanut intake (Vickery et al., JACI 2014, 133: 468-475). In many of the studies with peanut OIT, despite years of immunotherapy, subjects never achieve tolerance and are re-sensitized within weeks of halting daily peanut intake, with a small percentage (˜10%) maintaining a sustained unresponsiveness even after 3 months off OIT. Thus, there remains a new for new therapies for the treatment of food allergies such as peanut allergy.

### SUMMARY

Provided herein are methods for enhancing the efficacy, tolerability, and/or safety of a peanut or tree nut allergen-specific immunotherapy regimen in a subject having a peanut allergy and/or a tree nut allergy. In some embodiments, methods for enhancing the efficacy, tolerability, and/or safety of a peanut allergen-specific immunotherapy regimen in a subject having a peanut allergy are provided.

In one aspect, methods for enhancing the efficacy, safety, and/or tolerability of a peanut allergen immunotherapy regimen in a subject having a peanut allergy are provided. In some embodiments, the method comprises administering to the subject one or more doses of an interleukin-4 receptor (IL-4R) antagonist in combination with the immunotherapy regimen, wherein at least one dose of the IL-4R antagonist is administered prior to the start of the immunotherapy regimen. In some embodiments, the IL-4R antagonist is an anti-IL-4R antibody, or an antigen-binding fragment thereof, that comprises the heavy chain complementarity determining regions (HCDRs) of a heavy chain variable region (HCVR) comprising the amino acid sequence of SEQ ID NO:1 and the light chain complementarity determining regions (LCDRs) of a light chain variable region (LCVR) comprising the amino acid sequence of SEQ ID NO:2.

In some embodiments, the immunotherapy regimen is an oral immunotherapy (OIT) regimen.

In some embodiments, the peanut allergen is a composition comprising peanut flour.

In some embodiments, the immunotherapy regimen comprises an up-dosing phase followed by a maintenance phase, wherein the up-dosing phase comprises administering increasing doses of the peanut allergen over a period of at least 24 weeks and wherein the maintenance phase comprises administering one or more maintenance doses of the peanut allergen at the highest dose administered during the up-dosing phase. In some embodiments, the up-dosing phase comprises an initial dose escalation day (IDED) regimen followed by increasing dose escalations every two weeks. In some embodiments, the up-dosing phase comprises up-dosing from an initial dose of 0.5 mg peanut protein to a dose of 300 mg peanut protein and wherein the maintenance phase comprises administering one or more maintenance doses at 300 mg peanut protein.

In some embodiments, the subject is aged 6 years old to <18 years old.

In some embodiments, the subject has one or more of the following baseline characteristics:

(a) a clinical history of allergy to peanuts or peanut-containing foods;

(b) experiences dose-limiting symptoms at or before a challenge dose of 100 mg peanut protein, or at a cumulative dose of ≤144 mg of peanut protein, in a double-blind, placebo-controlled food challenge (DBPCFC);

(c) has a serum IgE to peanut of ≥10 kUA/L; or

(d) has a skin prick test (SPT) to peanut ≥8 mm.

In some embodiments, the subject has concomitant atopic dermatitis, asthma, eosinophilic esophagitis, and/or multiple food allergies.

In some embodiments, the IL-4R antagonist is administered at a dose of about 50 mg to about 600 mg. In some embodiments, the IL-4R antagonist is administered as an initial dose followed by one or more secondary doses, wherein each secondary dose is administered 1 to 4 weeks after the immediately preceding dose. In some embodiments, each secondary dose of the IL-4R antagonist is administered two weeks after the immediately preceding dose.

In some embodiments, the IL-4R antagonist is administered subcutaneously or intravenously.

In some embodiments, the IL-4R antagonist is subcutaneously administered at an initial dose followed by one or more secondary doses, wherein each secondary dose is administered 1 to 4 weeks after the immediately preceding dose, and wherein:

(i) for a subject weighing <30 kg, the initial dose of the IL-4R antagonist is 200 mg and each secondary dose is 100 mg; or

(ii) for a subject having a body weight of ≥30 kg to <60 kg, the initial dose of the IL-4R antagonist is 400 mg and each secondary dose is 200 mg; or

(iii) for a subject having a body weight of ≥60 kg, the initial dose of the IL-4R antagonist is 600 mg and each secondary dose is 300 mg.

In some embodiments, the subject has a body weight of <30 kg and the IL-4R antagonist is administered at an initial dose of 200 mg followed by one or more secondary doses of 100 mg every two weeks (Q2W). In some embodiments, the subject has a body weight of ≥30 kg to <60 mg and the IL-4R antagonist is administered at an initial dose of 400 mg followed by one or more secondary doses of 200 mg every two weeks (Q2W). In some embodiments, the subject has a body weight of ≥60 mg and the IL-4R antagonist is administered at an initial dose of 600 mg followed by one or more secondary doses of 300 mg every two weeks (Q2W).

In some embodiments, the initial dose of the IL-4R antagonist is administered at least two weeks before the start of the immunotherapy regimen. In some embodiments, the IL-4R antagonist is administered for at least four weeks before the start of the immunotherapy regimen.

In some embodiments, treatment with the IL-4R antagonist:

(i) increases the cumulative tolerated dose of peanut protein as measured by a DBPCFC; and/or

(ii) decreases the frequency and/or severity of peanut allergic symptoms, gastrointestinal symptoms, and/or itching symptoms.

In another aspect, methods for enhancing the efficacy, safety, and/or tolerability of a tree nut allergen immunotherapy regimen in a subject having a tree nut allergy are provided. In some embodiments, the tree nut is almond, brazil nut, cashew, hazelnut, pecan, pistachio, or walnut. In some embodiments, the method comprises administering to the subject one or more doses of an interleukin-4 receptor (IL-4R) antagonist in combination with the immunotherapy regimen, wherein at least one dose of the IL-4R antagonist is administered prior to the start of the immunotherapy regimen. In some embodiments, the IL-4R antagonist is an anti-IL-4R antibody, or an antigen-binding fragment thereof, that comprises the heavy chain complementarity determining regions (HCDRs) of a heavy chain variable region (HCVR) comprising the amino acid sequence of SEQ ID NO:1 and the light chain complementarity determining regions (LCDRs) of a light chain variable region (LCVR) comprising the amino acid sequence of SEQ ID NO:2.

In some embodiments, the anti-IL-4R antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO:3; the HCDR2 comprises the amino acid sequence of SEQ ID NO:4; the HCDR3 comprises the amino acid sequence of SEQ ID NO:5; the LCDR1 comprises the amino acid sequence of SEQ ID NO:6; the LCDR2 comprises the amino acid sequence of SEQ ID NO:7; and the LCDR3 comprises the amino acid sequence of SEQ ID NO:8. In some embodiments, the anti-IL-4R antibody or antigen-binding fragment thereof comprises a HCVR comprising the amino acid sequence of SEQ ID NO:1 and comprises a LCVR comprising the amino acid sequence of SEQ ID NO:2. In some embodiments, the anti-IL-4R antibody comprises a heavy chain comprising the amino acid sequence of SEQ ID NO:9 and a light chain comprising the amino acid sequence of SEQ ID NO:10. In some embodiments, the IL-4R antagonist is dupilumab.

In some embodiments, the IL-4R antagonist is contained in a container selected from the group consisting of a glass vial, a syringe, a pre-filled syringe, a pen delivery device, and an autoinjector. In some embodiments, the IL-4R antagonist is contained in a pre-filled syringe. In some embodiments, the pre-filled syringe is a single-dose pre-filled syringe. In some embodiments, the IL-4R antagonist is contained in an autoinjector.

Other embodiments will be apparent from a review of the ensuing detailed description.

### BRIEF DESCRIPTION OF THE DRAWINGS

FIG. 1. Schematic of study design for clinical trial disclosed in Example 1. Enroll 2-arms (2:1), up-dose AR101 (peanut oral immunotherapy) from week 4 to week 28 to 40. Subjects in DUP (dupilumab)+AR101 group will be re-randomized to DUP+AR101 and PBO (placebo)+AR101, but only those subjects who achieve 300 mg/day AR101 for at least 2 weeks will be eligible to enter the 24-week maintenance treatment. Subjects who do not achieve 300 mg/day AR101 will enter a 12-week follow-up. Abbreviations: DBPCFC, double-blind placebo-controlled food challenge; DUP, dupilumab; Exc, exclusion; Inc, inclusion; PBO, placebo.

FIG. 2. Frequency and severity of allergic reactions during the clinical trial Visit 16 DBPCFC. Left panel: percentage of subjects having symptoms in the dupilumab+AR101 group at each of challenge doses 1 mg, 3 mg, 10 mg, 30 mg, 100 mg, 300 mg, 600 mg, and 1000 mg. Right panel: percentage of subjects having symptoms in the placebo+AR101 group at each of challenge doses 1 mg, 3 mg, 10 mg, 30 mg, 100 mg, 300 mg, 600 mg, and 1000 mg.

### DETAILED DESCRIPTION

Before the present invention is described, it is to be understood that the invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

As used herein, the terms “treat,” “treating,” or the like, mean to alleviate symptoms, eliminate the causation of symptoms either on a temporary or permanent basis, or to prevent or slow the appearance of symptoms of the named disorder or condition.

The terms “prevent,” “preventing,” or the like, as used with reference to an allergic reaction or allergic condition, refer to preventing development of allergy, an allergic reaction, or an allergic condition. The term, as used herein, also includes reducing or abrogating allergen sensitization to prevent an allergic reaction. In some embodiments, the term refers to decreasing the level of serum allergen-specific IgE by at least 10%, at least 20%, at least 30%, at least 40%, or at least 50%, as compared to baseline, upon administration of an IL-4R antagonist as provided by the methods of the present disclosure.

As used herein, the term “subject in need thereof” refers to a human or non-human animal that (i) exhibits one or more symptoms or indicia of allergy, (ii) has been diagnosed with allergy to an allergen; and/or (iii) is at an increased risk for developing an allergy or an allergic response to an allergen. In certain embodiments, the term includes subjects that show allergen sensitization to one or more allergens (e.g., one or more peanut allergen components). In certain embodiments, the methods of the present disclosure may be used to treat subjects that show elevated levels of one or more serum biomarkers including, but not limited to, total IgE, allergen-specific IgE, thymus and activation-regulated chemokine (TARC), pulmonary and activation-regulated chemokine (PARC), lactate dehydrogenase (LDH), and periostin. For example, in some embodiments, the methods of the present disclosure comprise administering an IL-4R antagonist to patients with elevated levels of allergen-specific IgE. The terms “subject” and “patient” are used interchangeably herein.

As used herein, the terms “allergic response,” “allergic reaction,” “allergic symptom,” and the like, include one or more signs or symptoms selected from the group consisting of urticaria (e.g., hives), angioedema, rhinitis, asthma, vomiting, sneezing, runny nose, sinus inflammation, watery eyes, wheezing, bronchospasm, reduced peak expiratory flow (PEF), gastrointestinal distress, flushing, swollen lips, swollen tongue, reduced blood pressure, anaphylaxis, and organ dysfunction/failure. An “allergic response,” “allergic reaction,” “allergic symptom,” etc., also includes immunological responses and reactions such as, e.g., increased IgE production and/or increased allergen-specific immunoglobulin production.

The term “allergen,” refers to a substance, chemical, particle or composition that is capable of stimulating an allergic response in a susceptible individual. Allergens may be contained within or derived from a food item such as, e.g., dairy products (e.g., cow's milk), egg, celery, seeds (e.g., sesame), wheat, soy, fish, shellfish, sugars (e.g., sugars present on meat such as alpha-galactose), peanuts, other legumes (e.g., beans, peas, soybeans, etc.), and tree nuts (e.g., almonds, brazil nuts, cashews, hazelnuts, pecans, pistachios, and walnuts). Alternatively, an allergen may be contained within or derived from a non-food item such as, e.g., dust (e.g., containing dust mite), pollen, insect venom (e.g., venom of bees, wasps, mosquitos, fire ants, etc.), mold, animal fur, animal dander, wool, latex, metals (e.g., nickel), household cleaners, detergents, medication, cosmetics (e.g., perfumes, etc.), drugs (e.g., penicillin, sulfonamides, salicylate, etc.), therapeutic monoclonal antibodies (e.g., cetuximab), ragweed, grass and birch. In some embodiments, an allergen is contained within or derived from peanut. In some embodiments, the allergen is a peanut protein allergen component such as but not limited to Ara h 1, Ara h 2, or Ara h 3. The terms “allergen” and “antigen” are used interchangeably through the disclosure.

Although any methods and materials similar or equivalent to those described herein can be used in the practice of the invention, the typical methods and materials are now described. All publications mentioned herein are incorporated herein by reference in their entirety.

### Introduction

Provided herein are methods for enhancing the efficacy, tolerability, and/or safety of an allergen-specific immunotherapy in a patient having an allergy one or more doses of an interleukin-4 receptor (IL-4R) antagonist prior to or concurrent with the allergen-specific immunotherapy. In some embodiments, the use of an IL-4R antagonist (e.g., dupilumab or a bioequivalent thereof) may significantly enhance the success of allergen-specific immunotherapy such as peanut allergy immunotherapy by enhancing the tolerability of up-dosing to the allergen, speeding up the immunotherapy process, allowing greater protection against accidental allergen exposure in a shorter period of time, and/or improving the durable immune tolerance after the allergen-specific immunotherapy is discontinued.

### Therapeutic Methods

In one aspect, methods for enhancing the efficacy, tolerability and/or safety of a peanut allergen-specific immunotherapy regimen are provided. In some embodiments, the methods comprise administering to a subject having a peanut allergy one or more doses of an interleukin-4 receptor (IL-4R) antagonist prior to or concurrent with the immunotherapy regimen.

In another aspect, methods for enhancing the efficacy, tolerability and/or safety of a tree nut (e.g., almond, brazil nut, cashew, hazelnut, pecan, pistachio, or walnut) allergen-specific immunotherapy regimen are provided. In some embodiments, the methods comprise administering to a subject having a tree nut allergy one or more doses of an interleukin-4 receptor (IL-4R) antagonist prior to or concurrent with the immunotherapy regimen.

As used herein, “allergen-specific immunotherapy” refers to the repeated administration of gradually increasing doses of an allergen to a subject over time in order to induce immunologic tolerance to the allergen in the subject. In some embodiments, the allergen-specific immunotherapy comprises administering a peanut or tree nut allergen (e.g., whole peanut or tree nut; a protein, extract, or component isolated, purified, or derived from the peanut or tree nut; or a foodstuff comprising the peanut or tree nut, such as nut butter or nut flour). In some embodiments, the allergen-specific immunotherapy comprises administering a peanut allergen, e.g., peanut protein, or a composition comprising peanut protein, peanut extract, peanut allergen, or a peanut allergen component (e.g., Ara h 1, Ara h 2, or Ara h 3). In some embodiments, the immunotherapy comprises administering peanut (e.g., whole peanut or a portion thereof), peanut butter, peanut extract, or peanut flour or a composition comprising peanut, peanut butter, or peanut flour. In some embodiments, the immunotherapy (e.g., peanut allergen-specific immunotherapy) is oral immunotherapy, subcutaneous immunotherapy, epicutaneous immunotherapy, or sublingual immunotherapy.

An immunotherapy regimen can be a “conventional” immunotherapy regimen or an “accelerated” immunotherapy regimen. In some embodiments, the IL-4R antagonist is administered prior to or concurrent with a conventional immunotherapy regimen. Typically in conventional immunotherapy, increasing doses of the allergen are administered to the patient at weekly intervals over the course of several weeks to months (e.g., over 1, 2, 3, 4, 5, 6, 7, 8, 9, 10, 11, or 12 months or longer), under tightly monitored medical supervision.

In some embodiments, an IL-4R antagonist as disclosed herein is administered prior to or concurrent with an accelerated immunotherapy regimen. Accelerated immunotherapy regimens accelerate the up-dosing schedule of the immunotherapy as compared to conventional immunotherapy, and include “rush immunotherapy” and “cluster immunotherapy.” Typically in rush immunotherapy, increasing dosages of the allergen are administered per day over several consecutive days (e.g., over 2 days, 3 days, 4 days, 5 days, 6 days, or one week) until the maximum tolerated dose is reached. In cluster immunotherapy, typically several (e.g., 2-3) increasing dosages of the allergen are administered in a single day, over nonconsecutive days until the maximum tolerated dose is reached, usually within 4 to 8 weeks. In some embodiments, an IL-4R antagonist is administered prior to and/or concurrent with a conventional immunotherapy regimen. In some embodiments, the IL-4R antagonist is administered prior to and/or concurrent with a cluster immunotherapy regimen. In some embodiments, the IL-4R antagonist is administered prior to and/or concurrent with a rush immunotherapy regimen.

In some embodiments, the allergen-specific (e.g., peanut-specific) immunotherapy is an oral immunotherapy. As used herein, “oral immunotherapy” or “OIT” refers to the repeated oral administration of an allergen to a subject over time as means for treating or preventing allergies and allergic reactions, or to reduce or eliminate allergic responses. Typically, OIT involves orally administering gradually increasing quantities of an allergen to the subject until a dose is reached that is effective in inducing immunologic tolerance to the allergen. In some embodiments, the OIT comprises administering a composition comprising a peanut or tree nut allergen (e.g., a composition comprising peanut (Arachis hypogaea) allergen flour). In some embodiments, the OIT comprises administering PALFORZIA (Aimmune Therapeutics, Inc., Brisbane, Calif.). Exemplary compositions for peanut-specific immunotherapy (e.g., OIT) are disclosed in U.S. Pat. Nos. 9,198,869 and 9,492,535, incorporated by reference herein.

In some embodiments, the immunotherapy (e.g., OIT) comprises an up-dosing regimen, followed by a maintenance regimen. Generally, the up-dosing regimen comprises administering increasing doses of the allergen over a period of time until an effective and safe dose is achieved, and the maintenance regimen comprises administering one or more doses of the allergen at the highest dose administered during the up-dosing regimen. In some embodiments, the up-dosing regimen comprises an initial dose escalation period (e.g., over the course of a single day) followed by a subsequent dose escalation period (e.g., escalating doses every week or every two weeks). An exemplary up-dosing regimen comprising initial dose escalation and subsequent bi-weekly dose escalation is disclosed in Example 1 and at Tables 1-2.

In some embodiments, the OIT regimen comprises administering a composition comprising a peanut allergen in a dosing regimen that comprises (i) up-dosing over a single day from 0.5 mg peanut allergen to a maximum of 6 mg peanut protein (e.g., 1.5, 3, or 6 mg), (ii) up-dosing for at least 22 weeks (e.g., increasing the dosage weekly or every other week for at least about 24 or 28 weeks, or for about 22, 24, 26, 28, 30, 32, or 34 weeks) starting from the maximum single day dose (e.g., 1.5, 3, or 6 mg) to a dose of up to 300 mg peanut allergen (e.g., up to a dose of 120 mg, 160 mg, 200 mg, 240 mg, or 300 mg), and (iii) dosing at a maintenance dose that is equal to the highest tolerated dose from (ii) (e.g., a dose of 120 mg, 160 mg, 200 mg, 240 mg, or 300 mg). In some embodiments, the maintenance dose (e.g., a dose of 120 mg, 160 mg, 200 mg, 240 mg, or 300 mg) is administered daily for at least 2 weeks, at least 4 weeks, at least 6 weeks, at least 8 weeks, at least 12 weeks, or at least 16 weeks, or for at least 1 month, 2 months, 3 months, 4 months, 5 months, 6 months, 7 months, 8 months, 9 months, 10 months, 11 months, 12 months or longer.

In some embodiments, the efficacy, tolerability, and/or safety of an immunotherapy regimen is “enhanced” if one or more of the following outcomes or phenomena are observed or achieved in a subject: (1) the duration of the up-dosing phase is decreased without compromising efficacy or safety; (2) the duration of the maintenance phase is decreased without compromising efficacy or safety; (3) the number of doses of allergen administered during the up-dosing or maintenance phase is reduced without compromising efficacy or safety; (4) the frequency of allergen administration during the up-dosing or maintenance phase is reduced without compromising efficacy or safety; (5) the dose of allergen administered during the up-dosing or maintenance phase is increased without compromising efficacy or safety; (6) the frequency of allergic responses or adverse side-effects triggered by the immunotherapy regimen is reduced or eliminated; (7) the use of or need for conventional allergy medications (e.g., steroids, antihistamines, decongestants, anti-IgE agents, etc.) is reduced or eliminated during the up-dosing and/or maintenance phases; (8) the level of total IgE expression is reduced; (9) the level of allergen-specific IgG4 expression is increased; (10) the ratio of serum allergen-specific IgG4 to serum allergen-specific IgE is increased; (11) the frequency of anaphylactic reactions is reduced or eliminated; or (12) the need for a rescue medication (e.g., epinephrine or an oral steroid) is reduced or eliminated. In some embodiments, the efficacy of an immunotherapy regimen is “enhanced” if a subject experiences fewer and/or less severe allergic reactions following the immunotherapy regimen in combination with IL-4R blockade than with the immunotherapy regimen alone. In some embodiments, the efficacy of an immunotherapy regimen is “enhanced” if the maximum immunotherapy dose that is tolerated by the subject is increased when an IL-4R antagonist is administered, relative to immunotherapy alone. In some embodiments, the efficacy of an immunotherapy regimen is “enhanced” if there is a decreased need for rescue medication (e.g., epinephrine or oral steroids) for treating a systemic reaction when an IL-4R antagonist is administered, relative to immunotherapy alone.

In another aspect, methods are provided for treating, preventing or reducing the severity of an allergic reaction or allergic symptoms in a subject having a peanut or tree nut allergy by administering an IL-4R antagonist. In some embodiments, at least one dose of the IL-4R antagonist is administered prior to or concurrent with an immunotherapy regimen (e.g., OIT). In some embodiments, the IL-4R antagonist is administered for at least 1 week, at least 2 weeks, at least 3 weeks, or at least 4 weeks prior to the start of the immunotherapy regimen (e.g., OIT). In some embodiments, the IL-4R antagonist is administered for at least 4 weeks prior to the start of the immunotherapy regimen. In some embodiments, at least 1, 2, 3, 4 or more doses are administered to the subject prior to the start of the immunotherapy regimen. In some embodiments, the IL-4R antagonist is administered for at least 1 month, at least 2 months, at least 3 months, or at least 4 months prior to the start of the immunotherapy regimen (e.g., OIT).

In some embodiments, treatment with an IL-4R antagonist concurrent with an immunotherapy regimen improves a subject's desensitization to the allergen (e.g., peanut or tree nut). In some embodiments, treatment with an IL-4R antagonist concurrent with an immunotherapy regimen improves a subject's desensitization to peanut allergen (e.g., increases a subject's tolerance for peanut protein). As used herein, “desensitization,” as used with reference to a food allergen (e.g., peanut allergen), refers to the ability to tolerate a higher threshold of food allergen without an allergic reaction. In some embodiments, treatment with an IL-4R antagonist concurrent with an immunotherapy regimen (e.g., peanut OIT) improves a subject's desensitization to peanut allergen (e.g., to peanut protein or to one or more peanut protein allergen components, such as Ara h 1, Ara h 2, or Ara h 3) by at least 10%, 15%, 20%, 25%, 30%, 35%, 40%, 45%, 50%, 60%, 70%, 80%, 90%, or more relative to a baseline value for the subject (e.g., as measured in a double-blind placebo-controlled food challenge (DBPCFC) prior to the onset of treatment). In some embodiments, treatment with an IL-4R antagonist concurrent with an immunotherapy regimen (e.g., peanut OIT) improves a subject's desensitization to peanut allergen (e.g., to peanut protein or to one or more peanut protein allergen components, such as Ara h 1, Ara h 2, or Ara h 3) as measured by whether the subject can pass a DBPCFC (i.e., not exhibit any objective Grade 1 (mild) reaction by the CoFAR grading system) with 2044 mg cumulative peanut protein. In some embodiments, treatment with an IL-4R antagonist improves a subject's maintenance of desensitization to peanut allergen (e.g., following completion of an immunotherapy regimen).

In some embodiments, treatment increases the cumulative dose of peanut protein that the subject is able to tolerate, e.g., as measured in a double-blind placebo-controlled food challenge (DBPCFC) relative to a baseline value for the subject. In some embodiments, the baseline value is the cumulative dose of peanut protein that the subject is able to tolerate as measured in a DBPCFC prior to the start of treatment with the IL-4R inhibitor and/or the immunotherapy regimen. In some embodiments, treatment increases the cumulative dose of peanut protein that the subject is able to tolerate as measured in a DBPCFC at the completion of the immunotherapy regimen up-dosing phase. In some embodiments, treatment increases the cumulative dose of peanut protein that the subject is able to tolerate as measured in a DBPCFC at the start of, during, or at the completion of the immunotherapy regimen maintenance phase. In some embodiments, treatment results in a subject being able to maintain desensitization to the peanut allergen, e.g., as measured by whether the subject can pass a DBPCFC at or after the completion of the immunotherapy maintenance phase.

In some embodiments, the DBPCFC comprises administering increasing doses of peanut allergen to the subject, and monitoring the subject in between administered doses to determine whether the subject exhibits a reaction to the peanut allergen (e.g., as measured by the CoFAR grading system; see, e.g., Sampson et al., J Allergy Clin Immunol 2012, 130:1260-1274)). In some embodiments, the DBPCFC comprises administering increasing doses of peanut allergen up to a cumulative dose of at least 444 mg, at least 1044 mg, or at least 2044 mg, e.g., as shown in the schedule of dosing disclosed Table 3 below. In some embodiments, a subject who is treated with the IL-4R inhibitor is able to pass a DBPCFC with a cumulative dose of 444 mg at the completion of the immunotherapy regimen up-dosing phase or maintenance phase. In some embodiments, a subject who is treated with the IL-4R inhibitor is able to pass a DBPCFC with a cumulative dose of 1044 mg at the completion of the immunotherapy regimen up-dosing phase or maintenance phase. In some embodiments, a subject who is treated with the IL-4R inhibitor is able to pass a DBPCFC with a cumulative dose of 2044 mg at the completion of the immunotherapy regimen up-dosing phase or maintenance phase.

In some embodiments, treatment with an IL-4R antagonist concurrent with an immunotherapy regimen improves the efficacy, tolerability, and/or safety of an immunotherapy regimen as measured by an improvement in one or more biomarkers, e.g., a biomarker associated with Type 2 immune activity and/or an allergen-specific biomarker. In some embodiments, the biomarker is a serum biomarker. In some embodiments, the biomarker is total IgE, allergen-specific IgG4, or thymus and activation-regulated chemokine (TARC).

In some embodiments, the biomarker is a biomarker of Type 2 immune activity, such as but not limited to serum TARC or serum total IgE. In some embodiments, the biomarker is an allergen-specific biomarker, e.g., a peanut-specific biomarker, such as but not limited to peanut-specific IgE (e.g., serum peanut sIgE), peanut-specific IgG (e.g., serum peanut IgG), or peanut-specific IgG4 (e.g., serum peanut sIgG4). In some embodiments, the methods of the disclosure decrease the level of a Type 2 biomarker or inhibit the induction of a Type 2 biomarker by immunotherapy. In some embodiments, administration of the IL-4R antagonist reduces or inhibits a rise in sIgE that is induced during immunotherapy (e.g., during the up-dosing phase and/or the maintenance phase).

In some embodiments, the biomarker is peanut-specific IgG4 (e.g., serum peanut-specific sIgG4). Without being bound to a particular theory, it is hypothesized that the induction of allergen specific antibodies, especially of the IgG4 isotype, has a protective effect against IgE-mediated allergic symptoms, as IgG4 competes with IgE, blocking IgE-mediated effector cell activation, suppresses histamine release and inhibits antigen-presentation of IgE-allergen complex by dendritic and B-cells. In some embodiments, the methods of the disclosure increase the level of an allergen-specific biomarker (e.g., serum peanut allergen-specific IgG4) relative to a baseline for the subject or relative to a control value.

In some embodiments, both total IgE and allergen-specific IgG (e.g., IgG4) biomarkers are measured and a ratio of the allergen-specific IgG or IgG4 marker to the total IgE marker (e.g., a ratio of peanut allergen-specific IgG or IgG4 to total IgE) is calculated. In some embodiments, treatment with an IL-4R antagonist concurrent with a peanut immunotherapy regimen increases the ratio of allergen-specific IgG4 to total IgE in a sample from the subject, e.g., as compared to a baseline value for the subject or as compared to a control value (e.g., from a subject treated with peanut immunotherapy alone). In some embodiments, the methods of the disclosure increase the ratio of allergen-specific IgG4 to total IgE relative to a baseline for the subject or relative to a control value.

As will be appreciated by a person of ordinary skill in the art, an increase or decrease in a serum biomarker can be determined by comparing (i) the level of the biomarker measured in a subject at a defined time point after administration of the IL-4R antagonist to (ii) the level of the biomarker measured in the patient prior to the onset of treatment with the IL-4R antagonist (i.e., the “baseline measurement”). The defined time point at which the biomarker is measured can be, e.g., at about 4 hours, 8 hours, 12 hours, 1 day, 2 days, 3 days, 4 days, 5 days, 6 days, 7 days, 8 days, 9 days, 10 days, 15 days, 20 days, 35 days, 40 days, 50 days, 55 days, 60 days, 65 days, 70 days, 75 days, 80 days, 85 days, 100 days, 150 days, or more after the onset of treatment with the IL-4R antagonist.

Methods for detecting and/or quantifying a serum biomarker, such as allergen-specific IgE, total IgE, or TARC, are known in the art; kits for measuring such a biomarker are available from various commercial sources; and various commercial diagnostic laboratories offer services which provide measurement of such biomarkers as well.

For example, Phadiatop™ is a commercially available variant of serum specific or antigen-specific IgE assay test that was introduced for the screening of allergic sensitization (Merrett et al 1987, Allergy 17: 409-416). The test provides for simultaneous testing for serum specific IgE to a mixture of relevant allergens causing common inhalant allergies. The test gives a qualitative result, either positive or negative depending upon a fluorescence response obtained. When a patient sample gives a fluorescence response higher than or equal to the reference, a positive test result is indicated. A patient sample with a lower fluorescence response indicates a negative test result.

As another example, an exemplary assay system for measuring the level of the biomarker TARC is the TARC quantitative ELISA kit offered as Cat. No. DDN00 by R&D Systems, Minneapolis, Minn.

### Treatment Populations

The methods disclosed herein include administering to a subject in need thereof an IL-4R antagonist or a pharmaceutical composition comprising an IL-4R antagonist. In some embodiments, a subject in need of treatment according to the methods disclosed herein is a subject who exhibits one or more symptoms or indicia of a peanut allergy (e.g., an allergy to peanut, peanut-containing foods, and/or peanut allergen or peanut allergen components, such as Ara h1, Ara h2, or Ara h3), (ii) has been diagnosed with allergy to a peanut allergen; and/or (iii) is at an increased risk for developing a peanut allergy or an allergic response to a peanut allergen. In some embodiments, a subject in need of treatment according to the methods disclosed herein is a subject who exhibits one or more symptoms or indicia of a tree nut allergy (e.g., an allergy to a tree nut such as almond, brazil nut, cashew, hazelnut, pecan, pistachio, or walnut, foods containing the tree nut, and/or tree nut allergen components, such as Pru du6 (almond), Ber e1 or Ber e2 (brazil nut), Ano o1, Ano 02, or Ano o3 (cashew), Cor a1, Cor a9, Cor 11, or Cor a14 (hazelnut), Car 11 or Car 12 (pecan), Pis v1, Pis v2, or Pis v3 (pistachio), or Jug r1, Jug r2, or Jug r4 (walnut)), (ii) has been diagnosed with allergy to a tree nut allergen; and/or (iii) is at an increased risk for developing a tree nut allergy or an allergic response to a tree nut allergen. In some embodiments, the subject is a pediatric subject less than 18 years old. In some embodiments, a subject to be treated is at least 6 years old. In some embodiments, the subject is aged 6 to 17 years (inclusive). In some embodiments, a subject to be treated is an adult.

In some embodiments, the subject to be treated meets one or more of the following criteria: (a) a clinical history of allergy to peanuts or peanut-containing foods (e.g., exhibiting symptom(s) of reaction due to exposure); (b) experiences dose-limiting symptoms in a DBPCFC at or before the 100 mg challenge dose (≤144 mg cumulative) of peanut protein (e.g., measured as 200 mg of peanut flour) and does not experience dose-limiting symptoms to placebo; (c) serum IgE to peanut of ≥10 kUA/L; and (d) skin prick test (SPT) to peanut ≥8 mm compared to a negative control. In some embodiments, the subject to be treated tolerates ≤43 mg of peanut protein, e.g., as measured in a DBPCFC. In some embodiments, the subject to be treated has a baseline sIgE to peanut of >100 kUA/L, >150 kUA/L, or >200 kUA/L. In some embodiments, the subject to be treated has a baseline sIgE to peanut of >0.35 to ≤100 kUA/L. In some embodiments, the subject to be treated has a baseline sIgE to peanut of >0.35 to ≤52.5 kUA/L.

In some embodiments, the subject to be treated has one or more comorbid type 2 inflammatory diseases or conditions (e.g., atopic dermatitis, asthma, eosinophilic esophagitis, or allergic rhinitis). In some embodiments, the subject to be treated has a concomitant asthma and/or atopic dermatitis. In some embodiments, the subject to be treated has one or more other allergies (e.g., food allergies or non-food allergies). In some embodiments, the subject to be treated has a history of multiple food allergies. In some embodiments, the subject to be treated has a history of peanut anaphylaxis.

### Interleukin-4 Receptor Antagonists

In some embodiments, the methods of the present disclosure comprise administering to a subject in need thereof (e.g., a subject having a peanut allergy) an interleukin-4 receptor (IL-4R) antagonist or a pharmaceutical composition comprising an IL-4R antagonist. As used herein, an “IL-4R antagonist” (also referred to herein as an “IL-4R inhibitor”, an “IL-4R blocker,” or an “IL-4Rα antagonist”) is any agent that binds to or interacts with IL-4Rα or an IL-4R ligand, and inhibits or attenuates the normal biological signaling function of a type 1 and/or a type 2 IL-4 receptor. Human IL-4Rα has the amino acid sequence of SEQ ID NO:11. A type 1 IL-4 receptor is a dimeric receptor comprising an IL-4Rα chain and a γc chain. A type 2 IL-4 receptor is a dimeric receptor comprising an IL-4Rα chain and an IL-13Rα1 chain. Type 1 IL-4 receptors interact with and are stimulated by IL-4, while type 2 IL-4 receptors interact with and are stimulated by both IL-4 and IL-13. Thus, the IL-4R antagonists that can be used in the methods of the present disclosure may function by blocking IL-4-mediated signaling, IL-13-mediated signaling, or both IL-4- and IL-13-mediated signaling. The IL-4R antagonists of the present disclosure may thus prevent the interaction of IL-4 and/or IL-13 with a type 1 or type 2 receptor.

Non-limiting examples of categories of IL-4R antagonists include small molecule IL-4R inhibitors, anti-IL-4R aptamers, peptide-based IL-4R inhibitors (e.g., “peptibody” molecules), “receptor-bodies” (e.g., engineered molecules comprising the ligand-binding domain of an IL-4R component), and antibodies or antigen-binding fragments of antibodies that specifically bind human IL-4Rα. As used herein, IL-4R antagonists also include antigen-binding proteins that specifically bind IL-4 and/or IL-13.

### Anti-IL-4Rα Antibodies and Antigen-Binding Fragments Thereof

In certain exemplary embodiments of the present disclosure, the IL-4R antagonist is an anti-IL-4Rα antibody or antigen-binding fragment thereof. The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In some embodiments, the FRs of the anti-IL-4R antibody (or antigen-binding portion thereof) are identical to the human germline sequences. In some embodiments, one or more FRs of the anti-IL-4R antibody (or antigen-binding portion thereof) are naturally or artificially modified.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed by the term “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2, (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The constant region of an antibody is important in the ability of an antibody to fix complement and mediate cell-dependent cytotoxicity. Thus, in some embodiments the isotype of an antibody may be selected on the basis of whether it is desirable for the antibody to mediate cytotoxicity.

The term “antibody,” as used herein, also includes multispecific (e.g., bispecific) antibodies. A multispecific antibody or antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antibody or antigen-binding fragment of an antibody of the present disclosure using routine techniques available in the art. For example, in some embodiments the methods of the present disclosure comprise the use of bispecific antibodies wherein one arm of an immunoglobulin is specific for IL-4Rα or a fragment thereof, and the other arm of the immunoglobulin is specific for a second therapeutic target or is conjugated to a therapeutic moiety. Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

In some embodiments, the antibodies used in the methods of the present disclosure are human antibodies. The term “human antibody,” as used herein, is intended to include antibodies having variable and constant regions derived from human germline immunoglobulin sequences. The human antibodies of the disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods of the present disclosure may be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see, e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

An “isolated antibody” refers to an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody.” An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

According to certain embodiments, the antibodies used in the methods of the present disclosure specifically bind IL-4Rα. The term “specifically binds,” as used herein, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. In some embodiments, an antibody that “specifically binds” IL-4Rα binds to IL-4Rα or a portion thereof with an equilibrium dissociation constant (K_{D}) of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 1 nM, less than about 0.5 nM, less than about 0.25 nM, less than about 0.1 nM or less than about 0.05 nM, as measured in a surface plasmon resonance assay (e.g., BIAcore™, Cytiva, Marlborough, Mass.). In some embodiments, an antibody that specifically binds to a target antigen (e.g., IL-4Rα) can also specifically bind to another antigen, e.g., an ortholog of the target antigen. For example, in some embodiments, an isolated antibody that specifically binds human IL-4Rα exhibits cross-reactivity to other antigens, such as IL-4Rα molecules from other (non-human) species.

In some embodiments, the IL-4R antagonist is an anti-IL-4Rα antibody, or antigen-binding fragment thereof, comprising a heavy chain variable region (HCVR), light chain variable region (LCVR), and/or complementarity determining regions (CDRs) comprising any of the amino acid sequences of the anti-IL-4R antibodies as set forth in U.S. Pat. No. 7,608,693. In some embodiments, the IL-4R antagonist is an anti-IL-4Rα antibody or antigen-binding fragment thereof that comprises the heavy chain complementarity determining regions (HCDRs) of a heavy chain variable region (HCVR) comprising the amino acid sequence of SEQ ID NO:1 and the light chain complementarity determining regions (LCDRs) of a light chain variable region (LCVR) comprising the amino acid sequence of SEQ ID NO:2. In some embodiments, the IL-4R antagonist is an anti-IL-4Rα antibody or antigen-binding fragment thereof that comprises three HCDRs (HCDR1, HCDR2 and HCDR3) and three LCDRs (LCDR1, LCDR2 and LCDR3), wherein the HCDR1 comprises the amino acid sequence of SEQ ID NO:3, the HCDR2 comprises the amino acid sequence of SEQ ID NO:4, the HCDR3 comprises the amino acid sequence of SEQ ID NO:5, the LCDR1 comprises the amino acid sequence of SEQ ID NO:6, the LCDR2 comprises the amino acid sequence of SEQ ID NO:7, and the LCDR3 comprises the amino acid sequence of SEQ ID NO:8.

In some embodiments, the anti-IL-4R antibody or antigen-binding fragment thereof comprises the HCDR1, HCDR2, HCDR3, LCDR1, LCDR2, and LCDR3 of SEQ ID NOs:3, 4, 5, 6, 7, and 8, respectively, and further comprises an HCVR having at least 85% sequence identity (e.g., at least 90%, 91%, 92%, 93%, 94%, 95%, 96%, 97%, 98%, or 99% sequence identity) to the amino acid sequence of SEQ ID NO:1 and an LCVR having at least 85% sequence identity (e.g., at least 90%, 91%, 92%, 93%, 94%, 95%, 96%, 97%, 98%, or 99% sequence identity) to the amino acid sequence of SEQ ID NO:2. In some embodiments, the anti-IL-4R antibody or antigen-binding fragment thereof comprises an HCVR comprising SEQ ID NO:1 and an LCVR comprising SEQ ID NO:2.

In some embodiments, the anti-IL-4R antibody comprises a heavy chain comprising the amino acid sequence of SEQ ID NO:9. In some embodiments, the anti-IL-4R antibody comprises a light chain comprising the amino acid sequence of SEQ ID NO:10.

An exemplary antibody comprising a heavy chain comprising the amino acid sequence of SEQ ID NO:9 and a light chain comprising the amino acid sequence of SEQ ID NO:10 is the fully human anti-IL-4R antibody known as dupilumab. According to certain exemplary embodiments, the methods of the present disclosure comprise the use of dupilumab. As used herein, “dupilumab” also includes bioequivalents of dupilumab. The term “bioequivalent,” as used herein with reference to dupilumab, refers to anti-IL-4R antibodies or IL-4R-binding proteins or fragments thereof that are pharmaceutical equivalents or pharmaceutical alternatives whose rate and/or extent of absorption do not show a significant difference with that of dupilumab when administered at the same molar dose under similar experimental conditions, either single dose or multiple dose. In some embodiments, the term refers to antigen-binding proteins that bind to IL-4R which do not have clinically meaningful differences with dupilumab in their safety, purity and/or potency.

Other anti-IL-4Rα antibodies that can be used in the context of the methods of the present disclosure include, e.g., the antibody referred to and known in the art as AMG317 (Corren et al., 2010, Am J Respir Crit Care Med., 181(8):788-796), or MEDI 9314, or any of the anti-IL-4Rα antibodies as set forth in U.S. Pat. Nos. 7,186,809, 7,605,237, 7,638,606, 8,092,804, 8,679,487, 8,877,189, 10,774,141, or International Patent Publication No. WO2020/096381, the contents of each of which are incorporated by reference herein.

In some embodiments, an anti-IL-4Rα antibody or antigen-binding fragment thereof for use in the methods of the present disclosure comprises one or more CDR, HCVR, and/or LCVR sequences set forth in Table 13 below.

In some embodiments, an anti-IL-4Rα antibody comprises (i) an HCVR comprising the amino acid sequence of SEQ ID NO:32 (SCB-VH-59), SEQ ID NO:33 (SCB-VH-60), SEQ ID NO:34 (SCB-VH-61), SEQ ID NO:35 (SCB-VH-62), SEQ ID NO:36 (SCB-VH-63), SEQ ID NO:37 (SCB-VH-64), SEQ ID NO:38 (SCB-VH-65), SEQ ID NO:39 (SCB-VH-66), SEQ ID NO:40 (SCB-VH-67), SEQ ID NO:41 (SCB-VH-68), SEQ ID NO:42 (SCB-VH-69), SEQ ID NO:43 (SCB-VH-70), SEQ ID NO:44 (SCB-VH-71), SEQ ID NO:45 (SCB-VH-72), SEQ ID NO:46 (SCB-VH-73), SEQ ID NO:47 (SCB-VH-74), SEQ ID NO:48 (SCB-VH-75), SEQ ID NO:49 (SCB-VH-76), SEQ ID NO:50 (SCB-VH-77), SEQ ID NO:51 (SCB-VH-78), SEQ ID NO:52 (SCB-VH-79), SEQ ID NO:53 (SCB-VH-80), SEQ ID NO:54 (SCB-VH-81), SEQ ID NO:55 (SCB-VH-82), SEQ ID NO:56 (SCB-VH-83), SEQ ID NO:57 (SCB-VH-84), SEQ ID NO:58 (SCB-VH-85), SEQ ID NO:59 (SCB-VH-86), SEQ ID NO:60 (SCB-VH-87), SEQ ID NO:61 (SCB-VH-88), SEQ ID NO:62 (SCB-VH-89), SEQ ID NO:63 (SCB-VH-90), SEQ ID NO:64 (SCB-VH-91), SEQ ID NO:65 (SCB-VH-92), or SEQ ID NO:66 (SCB-VH-93); and (ii) an LCVR comprising the amino acid sequence of SEQ ID NO:12 (SCB-VL-39), SEQ ID NO:13 (SCB-VL-40), SEQ ID NO:14 (SCB-VL-41), SEQ ID NO:15 (SCB-VL-42), SEQ ID NO:16 (SCB-VL-43), SEQ ID NO:17 (SCB-VL-44), SEQ ID NO:18 (SCB-VL-45), SEQ ID NO:19 (SCB-VL-46), SEQ ID NO:20 (SCB-VL-47), SEQ ID NO:21 (SCB-VL-48), SEQ ID NO:22 (SCB-VL-49), SEQ ID NO:23 (SCB-VL-50), SEQ ID NO:24 (SCB-VL-51), SEQ ID NO:25 (SCB-VL-52), SEQ ID NO:26 (SCB-VL-53), SEQ ID NO:27 (SCB-VL-54), SEQ ID NO:28 (SCB-VL-55), SEQ ID NO:29 (SCB-VL-56), SEQ ID NO:30 (SCB-VL-57), or SEQ ID NO:31 (SCB-VL-58). In some embodiments, the anti-IL-4Rα antibody comprises an HCVR comprising the amino acid sequence of SEQ ID NO:64 (SCB-VH-91) and an LCVR comprising the amino acid sequence of SEQ ID NO:17 (SCB-VL-44), SEQ ID NO:27 (SCB-VL-54), or SEQ ID NO:28 (SCB-VL-55).

In some embodiments, an anti-IL-4Rα antibody comprises an amino acid sequence pair selected from the group consisting of: SEQ ID NOs:67/68 (MEDI-1-VH/MEDI-1-VL); SEQ ID NOs:69/70 (MEDI-2-VH/MEDI-2-VL); SEQ ID NOs:71/72 (MEDI-3-VH/MEDI-3-VL); SEQ ID NOs:73/74 (MEDI-4-VH/MEDI-4-VL); SEQ ID NOs:75/76 (MEDI-5-VH/MEDI-5-VL); SEQ ID NOs:77/78 (MEDI-6-VH/MEDI-6/VL); SEQ ID NOs:79/80 (MEDI-7-VH/MEDI-7-VL); SEQ ID NOs:81/82 (MEDI-8-VH/MEDI-8-VL); SEQ ID NOs:83/84 (MEDI-9-VH/MEDI-9-VL); SEQ ID NOs:85/86 (MEDI-10-VH/MEDI-10-VL); SEQ ID NOs:87/88 (MEDI-11-VH/MEDI-11/VL); SEQ ID NOs:89/90 (MEDI-12-VH/MEDI-12-VL); SEQ ID NOs:91/92 (MEDI-13-VH/MEDI-13-VL); SEQ ID NOs:93/94 (MEDI-14-VH/MEDI-14-VL); SEQ ID NOs:95/96 (MEDI-15-VH/MEDI-15-VL); SEQ ID NOs:97/98 (MEDI-16-VH/MEDI-16/VL); SEQ ID NOs:99/100 (MEDI-17-VH/MEDI-17-VL); SEQ ID NOs:101/102 (MEDI-18-VH/MEDI-18-VL); SEQ ID NOs:103/104 (MEDI-19-VH/MEDI-19-VL); SEQ ID NOs:105/106 (MEDI-20-VH/MEDI-20-VL); SEQ ID NOs:107/108 (MEDI-21-VH/MEDI-21-VL); SEQ ID NOs:109/110 (MEDI-22-VH/MEDI-22-VL); SEQ ID NOs:111/112 (MEDI-23-VH/MEDI-23-VL); SEQ ID NOs:113/114 (MEDI-24-VH/MEDI-24-VL); SEQ ID NOs:115/116 (MEDI-25-VH/MEDI-25-VL); SEQ ID NOs:117/118 (MEDI-26-VH/MEDI-26-VL); SEQ ID NOs:119/120 (MEDI-27-VH/MEDI-27-VL); SEQ ID NOs:121/122 (MEDI-28-VH/MEDI-28-VL); SEQ ID NOs:123/124 (MEDI-29-VH/MEDI-29-VL); SEQ ID NOs:125/126 (MEDI-30-VH/MEDI-30-VL); SEQ ID NOs:127/128 (MEDI-31-VH/MEDI-31-VL); SEQ ID NOs:129/130 (MEDI-32-VH/MEDI-32-VL); SEQ ID NOs:131/132 (MEDI-33-VH/MEDI-33-VL); SEQ ID NOs:133/134 (MEDI-34-VH/MEDI-34-VL); SEQ ID NOs:135/136 (MEDI-35-VH/MEDI-35-VL); SEQ ID NOs:137/138 (MEDI-36-VH/MEDI-36-VL); SEQ ID NOs:139/140 (MEDI-37-VH/MEDI-37-VL); SEQ ID NOs:141/142 (MEDI-38-VH/MEDI-38-VL); SEQ ID NOs:143/144 (MEDI-39-VH/MEDI-39-VL); SEQ ID NOs:145/146 (MEDI-40-VH/MEDI-40-VL); SEQ ID NOs:147/148 (MEDI-41-VH/MEDI-41-VL); SEQ ID NOs:149/150 (MEDI-42-VH/MEDI-42-VL); and SEQ ID NOs:151/152 (MEDI-37GL-VH/MEDI-37GL-VL).

In some embodiments, an anti-IL-4Rα antibody comprises (i) an HCVR comprising the amino acid sequence of SEQ ID NO:153 (AJOU-1-VH), SEQ ID NO:154 (AJOU-2-VH), SEQ ID NO:155 (AJOU-3-VH), SEQ ID NO:156 (AJOU-4-VH), SEQ ID NO:157 (AJOU-5-VH), SEQ ID NO:158 (AJOU-6-VH), SEQ ID NO:159 (AJOU-7-VH), SEQ ID NO:160 (AJOU-8-VH), SEQ ID NO:161 (AJOU-9-VH), SEQ ID NO:162 (AJOU-10-VH), SEQ ID NO:163 (AJOU-69-VH), SEQ ID NO:164 (AJOU-70-VH), SEQ ID NO:165 (AJOU-71-VH), SEQ ID NO:166 (AJOU-72-VH), or SEQ ID NO:167 (AJOU-83-VH); and (ii) an LCVR comprising the amino acid sequence of SEQ ID NO:168 (AJOU-33-VL), SEQ ID NO:169 (AJOU-34-VL), SEQ ID NO:170 (AJOU-35-VL), SEQ ID NO:171 (AJOU-36-VL), SEQ ID NO:172 (AJOU-37-VL), SEQ ID NO:173 (AJOU-38-VL), SEQ ID NO:174 (AJOU-39-VL), SEQ ID NO:175 (AJOU-40-VL), SEQ ID NO:176 (AJOU-41-VL), SEQ ID NO:177 (AJOU-42-VL), SEQ ID NO:178 (AJOU-77-VL), SEQ ID NO:179 (AJOU-78-VL), SEQ ID NO:180 (AJOU-79-VL), SEQ ID NO:181 (AJOU-80-VL), SEQ ID NO:182 (AJOU-86-VL), SEQ ID NO:183 (AJOU-87-VL), SEQ ID NO:184 (AJOU-88-VL), SEQ ID NO:185 (AJOU-89-VL), SEQ ID NO:186 (AJOU-90-VL), or SEQ ID NO:187 (AJOU-91-VL).

In some embodiments, an anti-IL-4Rα antibody comprises (i) an HCVR comprising the amino acid sequence of SEQ ID NO:188 (REGN-VH-3), SEQ ID NO:189 (REGN-VH-19), SEQ ID NO:190 (REGN-VH-35), SEQ ID NO:191 (REGN-VH-51), SEQ ID NO:192 (REGN-VH-67), SEQ ID NO:193 (REGN-VH-83), SEQ ID NO:194 (REGN-VH-99), SEQ ID NO:195 (REGN-VH-115), SEQ ID NO:196 (REGN-VH-147), or SEQ ID NO:197 (REGN-VH-163); and (ii) an LCVR comprising the amino acid sequence of SEQ ID NO:198 (REGN-VL-11), SEQ ID NO:199 (REGN-VL-27), SEQ ID NO:200 (REGN-VL-43), SEQ ID NO:201 (REGN-VL-59), SEQ ID NO:202 (REGN-VL-75), SEQ ID NO:203 (REGN-VL-91), SEQ ID NO:204 (REGN-VL-107), SEQ ID NO:205 (REGN-VL-123), SEQ ID NO:206 (REGN-VL-155), or SEQ ID NO:207 (REGN-VL-171).

In some embodiments, an anti-IL-4Rα antibody or antigen-binding fragment thereof used in the methods of the present disclosure can have pH-dependent binding characteristics. For example, an anti-IL-4Rα antibody for use as disclosed herein may exhibit reduced binding to IL-4Rα at acidic pH as compared to neutral pH. Alternatively, an anti-IL-4Rα antibody for use as disclosed herein may exhibit enhanced binding to its antigen at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding to IL-4Rα at acidic pH as compared to neutral pH” is expressed in terms of a ratio of the K_{D} value of the antibody binding to IL-4Rα at acidic pH to the K_{D} value of the antibody binding to IL-4Rα at neutral pH (or vice versa). For example, an antibody or antigen-binding fragment thereof may be regarded as exhibiting “reduced binding to IL-4Rα at acidic pH as compared to neutral pH” for purposes of the present disclosure if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present disclosure can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0, 25.0, 30.0, 40.0, 50.0, 60.0, 70.0, 100.0, or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained.

### Preparation of Human Antibodies

Methods for generating human antibodies in transgenic mice are known in the art. Any such known methods can be used in the context of the present disclosure to make human antibodies that specifically bind to human IL-4R.

Using VELOCIMMUNE™ technology (see, for example, U.S. Pat. No. 6,596,541, Regeneron Pharmaceuticals) or any other known method for generating monoclonal antibodies, high affinity chimeric antibodies to IL-4R are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc., using standard procedures known to those skilled in the art. The mouse constant regions are replaced with a desired human constant region to generate the fully human antibody of the disclosure, for example wild-type or modified IgG1 or IgG4. While the constant region selected may vary according to specific use, high affinity antigen-binding and target specificity characteristics reside in the variable region.

In general, the antibodies that can be used in the methods of the present disclosure possess high affinities, as described above, when measured by binding to antigen either immobilized on solid phase or in solution phase. The mouse constant regions are replaced with desired human constant regions to generate the fully human antibodies of the disclosure. While the constant region selected may vary according to specific use, high affinity antigen-binding and target specificity characteristics reside in the variable region.

In one embodiment, a human antibody or antigen-binding fragment thereof that specifically binds IL-4R and that can be used in the methods disclosed herein comprises the three heavy chain CDRs (HCDR1, HCDR2, and HCDR3) contained within a heavy chain variable region (HCVR) having an amino acid sequence of SEQ ID NO:1, and the three light chain CDRs (LCVR1, LCVR2, and LCVR3) contained within a light chain variable region (LCVR) having an amino acid sequence of SEQ ID NO:2. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

### Pharmaceutical Compositions

In one aspect, the present disclosure provides pharmaceutical compositions comprising an IL-4R antagonist (e.g., an anti-IL-4R antibody) for use in a method disclosed herein (e.g., for preventing or treating a peanut allergen, or for enhancing the efficacy, tolerability and/or safety of a peanut allergen-specific immunotherapy regimen) or for use in the manufacture of a medicament for preventing or treating a peanut allergen, or for enhancing the efficacy, tolerability and/or safety of a peanut allergen-specific immunotherapy regimen. In another aspect, the present disclosure provides therapeutic methods (e.g., for preventing or treating a peanut allergen, or for enhancing the efficacy, tolerability and/or safety of a peanut allergen-specific immunotherapy regimen) that comprise administering an IL-4R antagonist to a subject, wherein the IL-4R antagonist (e.g., an anti-IL-4R antibody) is contained within a pharmaceutical composition that comprises one or more pharmaceutically acceptable vehicle, carriers, and/or excipients. Various pharmaceutically acceptable carriers and excipients are well-known in the art. See, e.g., Remington's Pharmaceutical Sciences, Mack Publishing Company, Easton, Pa. In some embodiments, the carrier is suitable for intravenous, intramuscular, oral, intraperitoneal, intrathecal, transdermal, topical, or subcutaneous administration.

Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. In some embodiments, a pharmaceutical composition as disclosed herein is administered intravenously. In some embodiments, a pharmaceutical composition as disclosed herein is administered subcutaneously.

In some embodiments, the pharmaceutical composition comprises an injectable preparation, such as a dosage form for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

The dose of antibody administered to a subject according to the methods of the present disclosure may vary depending upon the age and the size of the subject, symptoms, conditions, route of administration, and the like. The dose is typically calculated according to body weight or body surface area. Depending on the severity of the condition, the frequency and the duration of the treatment can be adjusted. Effective dosages and schedules for administering pharmaceutical compositions comprising anti-IL-4R antibodies may be determined empirically; for example, subject progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351). Specific exemplary doses of anti-IL-4R antibodies, and administration regimens involving the same, that can be used in the context of the present disclosure are disclosed elsewhere herein.

In some embodiments, a pharmaceutical composition of the present disclosure is contained within a container. Thus, in another aspect, containers comprising a pharmaceutical composition as disclosed herein are provided. For example, in some embodiments, an IL-4R antagonist or a pharmaceutical composition comprising an IL-4R antagonist is contained within a container selected from the group consisting of a glass vial, a syringe, a pen delivery device, and an autoinjector.

In some embodiments, a pharmaceutical composition of the present disclosure is delivered, e.g., subcutaneously or intravenously, with a standard needle and syringe. In some embodiments, the syringe is a pre-filled syringe. In some embodiments, a pen delivery device or autoinjector is used to deliver a pharmaceutical composition of the present disclosure (e.g., for subcutaneous delivery). A pen delivery device can be reusable or disposable. Typically, a reusable pen delivery device utilizes a replaceable cartridge that contains a pharmaceutical composition. Once the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Examples of suitable pen and autoinjector delivery devices include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ 1, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™ OPTIPEN STARLET™, and OPTICLIK™ (sanofi-aventis, Frankfurt, Germany). Examples of disposable pen delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L.P.), and the HUMIRA™ Pen (Abbott Labs, Abbott Park Ill.).

In some embodiments, the pharmaceutical composition is delivered using a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533. Other delivery systems are known and can be used to administer the pharmaceutical composition, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432).

In some embodiments, the pharmaceutical composition is supplied in a container as disclosed herein (e.g., a glass vial, syringe, pen delivery device, or autoinjector) at a volume of about 0.5 mL to about 2.5 mL, e.g., about 0.7 mL, 0.8 mL, 0.9 mL, 1.0 mL, 1.1 mL, 1.15 mL, 1.2 mL, 1.25 mL, 1.3 mL, 1.4 mL, 1.5 mL, 1.6 mL, 1.7 mL, 1.75 mL, 1.8 mL, 1.9 mL, 2.0 mL, 2.1 mL, 2.2 mL, 2.25 mL, 2.3 mL, 2.4 mL, or 2.5 mL. In some embodiments, the pharmaceutical composition is contained in a volume of about 0.7 mL. In some embodiments, the pharmaceutical composition is contained in a volume of about 1.15 mL. In some embodiments, the pharmaceutical composition is contained in a volume of about 2.25 mL.

In some embodiments, pharmaceutical compositions for use as described herein are prepared into dosage forms in a unit dose suited to fit a dose of the active ingredients. Such dosage forms in a unit dose include, for example, tablets, pills, capsules, injections (ampoules), suppositories, etc.

In specific embodiments, the anti-IL-4R antibody comprises dupilumab. Unless otherwise specified, the term “dupilumab” also includes any biosimilars thereof.

Exemplary pharmaceutical compositions comprising an anti-IL-4R antibody that can be used in the context of the present disclosure are disclosed, e.g., in U.S. Pat. No. 8,945,559.

### Dosage and Administration Regimens

Typically, an IL-4R antagonist (e.g., an anti-IL-4R antibody as disclosed herein) is administered to a subject according to the methods of the present disclosure in a therapeutically effective amount. As used herein with reference to an IL-4R antagonist, the phrase “therapeutically effective amount” means an amount of IL-4R antagonist that results in one or more of: (a) treatment of or reduction in the severity or duration of an allergic reaction; (b) the alleviation of one or more symptoms or indicia of an allergic reaction; (c) increase in the ratio of serum allergen-specific IgG4 to serum allergen-specific IgE; (d) reduction in the level of one or more markers of Type 2 immune activity (e.g., serum TARC or total IgE); (e) reduction in the frequency of allergic responses to allergen-specific immunotherapy; and (f) increase in desensitization to peanut protein or peanut allergen (e.g., as measured in a double-blind placebo-controlled food challenge).

In the case of an anti-IL-4R antibody, a therapeutically effective amount can be from about 0.05 mg to about 600 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the anti-IL-4R antibody. In some embodiments, a therapeutically effective amount is from about 50 mg to about 600 mg or from about 75 mg to about 600 mg. In certain embodiments, 75 mg, 100 mg, 150 mg, 200 mg, or 300 mg of an anti-IL-4R antibody is administered to a subject.

The amount of IL-4R antagonist (e.g., anti-IL-4R antibody) contained within the individual doses may be expressed in terms of milligrams of antibody per kilogram of subject body weight (i.e., mg/kg). For example, the IL-4R antagonist may be administered to a subject at a dose of about 0.0001 to about 10 mg/kg of subject body weight, e.g., at a dose of about 1 mg/kg to about 10 mg/kg, or about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg.

In some embodiments, the methods disclosed herein comprise administering an IL-4R antagonist to a subject at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. In some embodiments, an anti-IL-4R antibody is administered once a week or one every two weeks.

In some embodiments, multiple doses of an IL-4R antagonist are administered to a subject over a defined time course. In some embodiments, the methods of the present disclosure comprise sequentially administering to a subject multiple doses of an IL-4R antagonist. As used herein, “sequentially administering” means that each dose of IL-4R antagonist is administered to the subject at a different point in time, e.g., on different days separated by a predetermined interval (e.g., hours, days, weeks or months). In some embodiments, the methods of the disclosure comprise sequentially administering to the patient a single initial dose of an IL-4R antagonist, followed by one or more secondary doses of the IL-4R antagonist, and optionally followed by one or more tertiary doses of the IL-4R antagonist.

The terms “initial dose,” “secondary doses,” and “tertiary doses,” refer to the temporal sequence of administration of the IL-4R antagonist. Thus, the “initial dose” is the dose which is administered at the beginning of the treatment regimen (also referred to as the “loading dose”); the “secondary doses” are the doses which are administered after the initial dose; and the “tertiary doses” are the doses which are administered after the secondary doses. The initial, secondary, and tertiary doses may all contain the same amount of IL-4R antagonist, but generally may differ from one another in terms of frequency of administration. In certain embodiments, however, the amount of IL-4R antagonist contained in the initial, secondary and/or tertiary doses varies from one another (e.g., adjusted up or down as appropriate) during the course of treatment. In certain embodiments, one or more (e.g., 1, 2, 3, 4, or 5) doses are administered at the beginning of the treatment regimen as “loading doses” followed by subsequent doses that are administered on a less frequent basis (e.g., “maintenance doses”).

In some embodiments, a loading dose is a “split dose” that is administered as two or more doses (e.g., 2, 3, 4, or 5 doses) that are administered on separate days. In some embodiments, a loading dose is administered as a split dose wherein the two or more doses are administered at least about one week apart. In some embodiments, a loading dose is administered as a split dose wherein the two or more doses are administered about 1 week, 2 weeks, 3 weeks, or 4 weeks apart. In some embodiments, the loading dose is split evenly over the two or more doses (e.g., half of the loading dose is administered as the first portion and half of the loading dose is administered as the second portion). In some embodiments, the loading dose is split unevenly over the two or more doses (e.g., more than half of the loading dose is administered as the first portion and less than half of the loading dose is administered as the second portion). In some embodiments, a loading dose is administered as a split dose wherein the first portion of the loading dose (e.g., the first half) is administered at Day 1 and the second portion of the loading dose (e.g., the second half) is administered after 1 week (e.g., on Day 8), after 2 weeks (e.g., on Day 15), after 3 weeks (e.g., on Day 22), or after 4 weeks (e.g., on Day 29), followed by one or more secondary or maintenance doses.

For example, an IL-4R antagonist may be administered to a subject at an initial or loading dose of about 200 mg, about 400 mg, or about 600 mg followed by one or more secondary or maintenance doses of about 75 mg to about 300 mg. In one embodiment, the initial dose and the one or more secondary doses each include 50 mg to 600 mg of the IL-4R antagonist, e.g., 100 mg, 150 mg, 200 mg, 250 mg, 300 mg, 400 mg, 500 mg, or 600 mg of the IL-4R antagonist. In some embodiments, the initial dose and the one or more secondary doses each contain the same amount of the IL-4R antagonist. In other embodiments, the initial dose comprises a first amount of the IL-4R antagonist, and the one or more secondary doses each comprise a second amount of the IL-4R antagonist. For example, the first amount of the IL-4R antagonist can be 1.5×, 2×, 2.5×, 3×, 3.5×, 4× or 5× or more than the second amount of the IL-4R antagonist. In one exemplary embodiment, an IL-4R antagonist is administered to a subject at a loading dose of about 600 mg followed by one or more maintenance doses of about 300 mg. In another exemplary embodiment, an IL-4R antagonist is administered to a subject at a loading dose of about 400 mg followed by one or more maintenance doses of about 200 mg. In yet another exemplary embodiment, an IL-4R antagonist is administered to a subject at a loading dose of about 200 mg followed by one or more maintenance doses of about 100 mg. In some embodiments, a subject is administered an IL-4R antagonist (e.g., one or more doses from about 50 mg to about 600 mg, e.g., about 50 mg, about 75 mg, about 100 mg, about 150 mg, about 200 mg, about 250 mg, about 300 mg, about 350 mg, about 400 mg, about 450 mg, about 500 mg, about 550 mg, or about mg) without a loading dose.

In some embodiments, each secondary and/or tertiary dose is administered 1 to 14 (e.g., 1, 1%, 2, 2%, 3, 3%, 4, 4%, 5, 5%, 6, 6%, 7, 7%, 8, 8%, 9, 9%, 10, 10%, 11, 11%, 12, 12%, 13, 13%, 14, 14%, or more) weeks after the immediately preceding dose. The phrase “the immediately preceding dose,” as used herein, means, in a sequence of multiple administrations, the dose of IL-4R antagonist which is administered to a patient prior to the administration of the very next dose in the sequence with no intervening doses.

The methods of the disclosure may comprise administering to a patient any number of secondary and/or tertiary doses of an IL-4R antagonist. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In some embodiments involving multiple secondary doses, each secondary dose is administered at the same frequency as the other secondary doses. For example, each secondary dose may be administered to the patient 1 to 2 weeks after the immediately preceding dose. Similarly, in some embodiments involving multiple tertiary doses, each tertiary dose is administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

In some embodiments, the IL-4R antagonist (e.g., anti-IL-4R antibody) is administered to the subject (e.g., a subject who is ≥6 years to <18 years of age) at a dose of 300 mg every weeks (Q2W), with or without a loading dose (e.g., a loading dose of 600 mg). In some embodiments, the IL-4R antagonist (e.g., anti-IL-4R antibody) is administered to the subject (e.g., a subject who is ≥6 years to <18 years of age) at a dose of 200 mg every weeks (Q2W), with or without a loading dose (e.g., a loading dose of 400 mg). In some embodiments, the IL-4R antagonist (e.g., anti-IL-4R antibody) is administered to the subject (e.g., a subject who is ≥6 years to <18 years of age) at a dose of 100 mg every weeks (Q2W), with or without a loading dose (e.g., a loading dose of 200 mg).

In some embodiments, for a subject having a peanut allergy (e.g., a subject who is ≥6 years to <18 years of age) weighing <30 kg, the IL-4R antagonist (e.g., anti-IL-4R antibody) is administered at a dose of 100 mg Q2W. In some embodiments, for a subject who is ≥6 years to <18 years of age) and weighing <30 kg, the IL-4R antagonist is administered at a loading dose followed by one or more maintenance doses, wherein the initial dose of the IL-4R antagonist comprises 200 mg and each maintenance dose comprises 100 mg, administered Q2W.

In some embodiments, for a subject having a peanut allergy (e.g., a subject who is >6 years to <18 years of age) weighing ≥30 kg to <60 kg, the IL-4R antagonist (e.g., anti-IL-4R antibody) is administered at a dose of 200 mg Q2W. In some embodiments, for a subject who is ≥6 years to <18 years of age) and weighing ≥30 kg to <60 kg, the IL-4R antagonist is administered at a loading dose followed by one or more maintenance doses, wherein the initial dose of the IL-4R antagonist comprises 400 mg and each maintenance dose comprises 200 mg, administered Q2W.

In some embodiments, for a subject having a peanut allergy (e.g., a subject who is ≥6 years to <18 years of age) weighing ≥60 kg, the IL-4R antagonist (e.g., anti-IL-4R antibody) is administered at a dose of 300 mg Q2W. In some embodiments, for a subject who is ≥6 years to <18 years of age) and weighing ≥60 kg, the IL-4R antagonist is administered at a loading dose followed by one or more maintenance doses, wherein the initial dose of the IL-4R antagonist comprises 600 mg and each maintenance dose comprises 300 mg, administered Q2W.

In some embodiments, immunotherapy is administered to the subject as an OIT regimen comprising an up-titration phase of at least 20 weeks, e.g., at least about 24 weeks, 26 weeks, 28 weeks, 30 weeks, 32 weeks, 34 weeks, or 36 weeks (e.g., from 20-40 weeks, from 24-40 weeks, or from 28-40 weeks) followed by a maintenance phase of 4, 6, 8, 10, 12 weeks or more.

In some embodiments, at least one dose of the IL-4R antagonist is administered one or more days before the start of the immunotherapy regimen. In some embodiments, at least one dose of the IL-4R antagonist is administered at least 1, 2, 3, 4, 5, 6, 7, 8, 9, 10 or more days before the start of the immunotherapy regimen. In some embodiments, the IL-4R antagonist is administered for at least 2 weeks, at least 3 weeks, at least 4 weeks, at least 5 weeks, or at least 6 weeks before the start of the immunotherapy regimen. In some embodiment, an initial (loading) dose and at least one secondary (maintenance) dose of the IL-4R antagonist are administered to the subject prior to the start of the immunotherapy regimen. In some embodiment, an initial (loading) dose and at least two secondary (maintenance) doses of the IL-4R antagonist are administered to the subject prior to the start of the immunotherapy regimen.

In some embodiments, the IL-4R antagonist and the immunotherapy are not administered to the subject on the same day. In some embodiments, the IL-4R antagonist and the immunotherapy are not administered to the subject within 24 hours, 18 hours, 12 hours, or 8 hours of each other.

### Combination Therapies

In some embodiments, the methods of the present disclosure comprise administering to the subject the IL-4R antagonist, or the IL-4R antagonist and the immunotherapy regimen, in combination with one or more additional therapeutic agents. As used herein, the expression “in combination with” means that the one or more additional therapeutic agents are administered before, concurrent with, or after the IL-4R antagonist or the IL-4R antagonist and the immunotherapy regimen.

For example, when administered “before” the pharmaceutical composition comprising the IL-4R antagonist, the additional therapeutic agent may be administered about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes prior to the administration of the pharmaceutical composition comprising the IL-4R antagonist. When administered “after” the pharmaceutical composition comprising the IL-4R antagonist, the additional therapeutic agent may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours or about 72 hours after the administration of the pharmaceutical composition comprising the IL-4R antagonist. Administration “concurrent” or with the pharmaceutical composition comprising the IL-4R antagonist means that the additional therapeutic agent is administered to the subject in a separate dosage form within about one day (e.g., within about 24 hours, about 12 hours, about 6 hours, about 3 hours, about 2 hours, about 1 hour, about 30 minutes, about 15 minutes, about 10 minutes, or about 5 minutes) before, after, or at the same time of administration of the pharmaceutical composition comprising the IL-4R antagonist, or administered to the subject as a single combined dosage formulation comprising both the additional therapeutic agent and the IL-4R antagonist.

In some embodiments, the additional therapeutic agent is a steroid, an antihistamine, a decongestant, or an anti-IgE agent. In some embodiments, the additional therapeutic agent is a steroid (e.g., a corticosteroid, such as an inhaled corticosteroid (ICS), oral corticosteroid (OCS), intravenous corticosteroid, intramuscular corticosteroid, or subcutaneous corticosteroid). In some embodiments, the additional therapeutic agent is an antihistamine (e.g., loratadine, fexofenadine, cetirizine, diphenhydramine, promethazine, carbinoxamine, desloratadine, hydroxyzine, levocetirizine, triprolidine, brompheniramine, or chlorpheniramine). In some embodiments, the additional therapeutic agent is a decongestant (e.g., pseudoephedrine or phenylephrine). In some embodiments, the additional therapeutic agent is an anti-IgE agent (e.g., omalizumab). In some embodiments, the additional therapeutic agent is an anti-IgE antibody (e.g., omalizumab or ligelizumab), an anti-IL-5 antibody (e.g., mepolizumab or reslizumab), or an anti-IL-5R antibody (e.g., benralizumab)

### EXAMPLES

The following examples are put forth so as to provide those of ordinary skill in the art with a complete disclosure and description of how to make and use the methods and compositions of the disclosure, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Clinical Trial Investigating the Efficacy of Dupilumab as an Adjunct to Peanut Oral Immunotherapy

### Study Design and Objectives

This is a Phase 2, multicenter, randomized, double-blind, parallel group, 2-arm study. of dupilumab as an adjunct to peanut oral immunotherapy (OIT) in subjects aged 6 to 17 years inclusive who are allergic to peanut. Dupilumab is a fully human anti-IL-4R antibody comprising a heavy chain comprising the amino acid sequence of SEQ ID NO:9 and a light chain comprising the amino acid sequence of SEQ ID NO:10; an HCVR/LCVR amino acid sequence pair comprising SEQ ID NOs:1/2; and heavy and light chain CDR sequences comprising SEQ ID NOs:3-8.

The primary objective of this study is to assess whether dupilumab as adjunct to the peanut oral immunotherapy AR101 compared to placebo improves desensitization at the completion of the up-dosing period, defined as an increase in the proportion of subjects who pass a post up-dosing double-blind placebo-controlled food challenge (DBPCFC) with 2044 mg (cumulative) peanut protein at visit 16.

The secondary objectives of this study include: (i) to assess whether dupilumab as adjunct to AR101 compared to placebo improves desensitization at the completion of up-dosing, defined as an increase in the cumulative tolerated dose (log transformed) of peanut protein during a post up-dosing DBPCFC from baseline to visit 16; (ii) to assess whether dupilumab as adjunct to AR101 compared to placebo maintains desensitization, defined as an increase in the proportion of subjects who pass a post maintenance DBPCFC at visit 22; (iii) to assess the proportion of subjects treated with dupilumab plus AR101 versus placebo plus AR101 who reach the 300 mg/day dose of AR101 for at least 2 weeks by visit 16; (iv) to assess the time from randomization to the first time when subjects reach the 300 mg/day dose of AR101 during the treatment phase (up to visit 16); (v) to evaluate the safety and tolerability of dupilumab as adjunct to AR101 compared to placebo; (vi) to assess the effect of dupilumab (compared to placebo) as adjunct to AR101 on the change and percent change from baseline to visit 16 in total IgE; (vii) to assess the effect of dupilumab (compared to placebo) as adjunct to AR101 on the change and percent change from baseline to visit 16 in peanut-specific IgE, IgG, and IgG4, and, and the change from baseline to visit 16 in log-transformed peanut-specific IgG/sIgE and IgG4/IgE ratios; (viii) to assess the effect of dupilumab (compared to placebo) as adjunct to AR101 on the change and percent change from baseline to visit 16 in Ara h1 sIgE, Ara h1 sIgG4, Ara h2 sIgE, Ara h2 sIgG4, Ara h3 sIgE, and Ara h3 sIgG4, and the change from baseline to visit 16 in log-transformed Ara h1-specific sIgG4/sIgE ratio, Ara h2-specific sIgG4/sIgE ratio, and Ara h3-specific sIgG4/sIgE ratio; (ix) to assess if dupilumab increases the tolerability of AR101 as measured by the daily symptoms (electronic diary [e-diary]) during the up-dosing phase; (x) to assess the change and percent change from baseline to visit 16 in basophil sensitivity to peanut allergen, as measured by EC50, which is the concentration of peanut protein required to achieve 50% of maximal basophil activation; and (xi) to measure the change and percent change from baseline to visit 16 in the frequency of peanut-specific T cell subsets (e.g., Th2A cells).

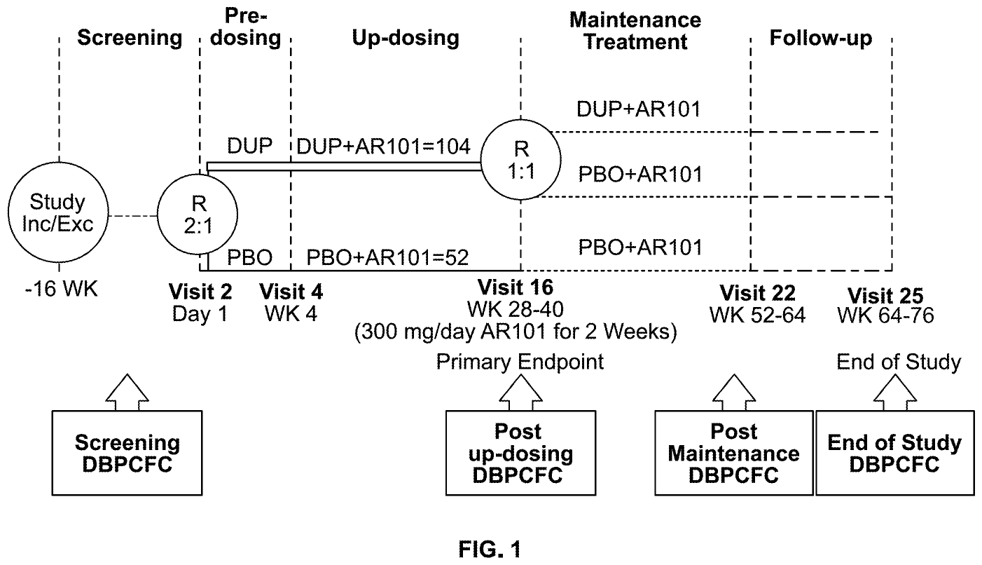

The study consists of a screening period of up to 16 weeks; a 28 to 40 week double-blind treatment period, which includes 4 weeks of pretreatment with dupilumab or placebo followed by 24 to 36 weeks of treatment with dupilumab or placebo in combination with a gradual up-dosing of AR101; a 24-week maintenance phase with 300 mg/day AR101 with concomitant dupilumab or matching placebo (for subjects who achieve 300 mg/day AR101 for at least 2 weeks in the up-dosing period); and a 12-week post-treatment follow-up period. Subjects who do not achieve 300 mg/day will still enter the 12-week follow-up. A schematic of the study design is shown in FIG. 1.

Screening: After obtaining informed consent, subjects are assessed for eligibility during a 3-part screening period as follows. During screening visit 1 (day −113 to day −17), subjects will undergo a medical history, physical examination, spirometry, peanut SPT, and laboratory testing (including peanut sIgE), and will be evaluated for the study eligibility criteria. Subjects must meet all eligibility for screening visit 1 before proceeding with the DBPCFC in visit 1a. During screening visit 1a (must be before day −15), under direct study-investigator monitoring, subjects will undergo a screening DBPCFC to confirm current peanut allergy. This will consist of 5 doses of peanut protein given every 15 to 30 minutes in increasing amounts up to a cumulative total of 144 mg of peanut protein. Vital signs will be assessed every 15 to 30 minutes. If the study team suspects a reaction may be developing, they may exercise their clinical judgement to separate doses by up to an additional 30 minutes (1-hour maximum between doses). The matching placebo challenge will consist of placebo material (oat protein) given also in 5 doses. The food challenges will be performed on different days (1-day placebo [oat] protein, 1-day peanut protein, with order determined at random) at least 24 hours apart. The doses will be 1, 3, 10, 30 and 100 mg of peanut protein (or placebo). Both food challenge days (placebo and peanut) must be done. Any subject who is assessed to have had dose-limiting symptoms to the placebo part, or both parts, of the screening DBPCFC (i.e., to oat flour as well as peanut flour) will be considered a screen failure and will not be randomized. Investigator/site personnel will be unblinded only to the results of the screening food challenge upon completion of the second part of the challenge to assess eligibility. All other food challenges in the study will remain blinded.

Double-blind treatment period (28 to 40 weeks duration): Subjects with a history of confirmed peanut allergy signs and symptoms who continue to meet eligibility criteria at baseline will undergo day 1/baseline assessments and will be randomized in a 2:1 ratio stratified by screening peanut-specific IgE level (5100 kUA/L or >100 kUA/L) and body weight (<30 kg, ≥30 kg and <60 kg, or ≥60 kg) at randomization into 1 of 2 treatment arms (n=52 for placebo and 104 for dupilumab). Dupilumab and placebo will be dosed SC as follows based on weight at randomization and dose will not be changed regardless of weight gain or loss:
subjects weighing <30 kg will receive dupilumab 100 mg Q2W following a loading dose of 200 mg on day 1 or matching placebo Q2W (including doubling the amount on day 1)
subjects weighing ≥30 kg and <60 kg will receive dupilumab 200 mg Q2W following a loading dose of 400 mg on day 1 or matching placebo Q2W (including doubling the amount on day 1)
subjects weighing ≥60 kg will receive dupilumab 300 mg Q2W following a loading dose of 600 mg on day 1 or matching placebo Q2W (including doubling the amount on day 1)

After the first 4 weeks of pretreatment, subjects will begin AR101 with an up-dosing regimen to a maximum of 300 mg/day over the next 24 to 36 weeks of the study, for a total of 28 to 40 weeks (including pretreatment), with 28 weeks being ideal and 40 weeks being the maximum. (The flexible up-dosing period is to accommodate dose reductions and re-escalation as well as COVID-19 restrictions of in-clinic visits. Thus, the 28 to 40 week up-dosing period will consist of 4 weeks pretreatment, 22 to 34 weeks of flexible up-dosing, and at least 2 weeks at the maximum dose of 300 mg/day.)

During concomitant AR101, study drug will be administered SC at home (at least 24 hours after in-clinic AR101 dose escalation and at least 8 hours apart from the at-home daily AR101 dose). After ingestion of AR101, subject should be monitored for 2 hours for allergic reaction. All subjects will complete up to visit 15 of the up-dosing period. Visits 15a-f will be conducted as needed in order to achieve 300 mg/day AR101 for 2 weeks. Subjects will undergo a post up-dosing DBPCFC at visit 16 if they have reached 300 mg/day for at least 2 weeks (on a day after the last dose of AR101). Dosing with AR101 should continue on the days between the 2 parts of the DBPCFC.

### Placebo Group

At day 1: Placebo (weight-based dose) Q2W SC for 28 to 40 weeks.

At week 4: AR101 using a standardized initial dose escalation day (IDED) regimen of doses of 0.5 mg to a maximum of 6 mg peanut protein (12 mg cumulative) over 5 hours in-clinic (home dosing will be 3 mg/day peanut protein for the next 2 weeks until up-dosing) (Table 1) followed by bi-weekly (every 2 weeks) in-clinic up-dosing from the highest tolerated initial day dose to a maximum of 300 mg/day at home for 22 to 34 additional weeks (Table 2). If the scheduled bi-weekly up-dosing is not possible, the up-dosing period may be extended by up to 12 weeks to accommodate dose reductions and re-escalation as well as COVID-19 restrictions of in-clinic visits. Thus, the up-dosing period will end at week 28 to 40 (including 4 weeks pretreatment, 22 to 34 weeks of flexible up-dosing, and at least 2 weeks at the maximum dose).

### Dupilumab Group

At day 1: Dupilumab (weight-based dose) Q2W SC for 28 to 40 weeks.

At week 4: AR101 using a standardized IDED regimen of doses of 0.5 mg to a maximum of 6 mg peanut protein (12 mg cumulative) over 5 hours in-clinic (home dosing will be 3 mg/day peanut protein for the next 2 weeks until up-dosing) (Table 1) followed by bi-weekly in-clinic up-dosing from the highest tolerated initial day dose to a maximum of 300 mg/day at home for 22 to 34 additional weeks (Table 2). If the scheduled bi-weekly up-dosing is not possible, the up-dosing period may be extended by up to 12 weeks to accommodate dose reductions and re-escalation as well as COVID-19 restrictions of in-clinic visits. Thus, the up-dosing period will end at week 28 to 40 (including 4 weeks pretreatment, 22 to 34 weeks of flexible up-dosing, and at least 2 weeks at the maximum dose).

Each AR101 dosing increase will be administered in-clinic and monitored for adverse allergic events for at least 2 hours prior to discharge. If the current dose is maintained (i.e., subject remains at the same dose and not escalated to the new dose during the clinic visit), then the observation period can be shortened to 1 hour with subject being discharged if deemed clinically stable. Vital signs will be monitored every 15 to 30 minutes. Subjects/legal guardians will be instructed not to exceed the specifically assigned doses at home. They will also be instructed not to introduce any new foods to the diet and to avoid accidental ingestions. Subjects/legal guardians will be supplied with epinephrine autoinjectors for treatment of allergic reactions and 24-hour access to an emergency contact telephone number. On the day following in-clinic AR101 up-dosing, the site is to make telephone contact with the subject/subject's parent or guardian to inquire if any AEs (including allergic symptoms) occurred subsequent to the subject leaving the clinic, and to provide assistance in the recording of any such events in the diary.

Subjects who exhibit moderate symptoms may have the dose reduced by 1 dose level per visit (i.e., go back to the previous dose prior to new up-dose) until the dose is tolerated with no or mild symptoms. A dose reduction, at the investigator's discretion, may also be implemented for an intercurrent adverse event. Subjects who exhibit severe allergic symptoms must be discontinued from study drug.

TABLE 1
AR101 Initial Dose Escalation Day at Week 4

| Study Week | Dupilumab/Placebo + AR101 |
|---|---|
| Week 4 (1 to 5 h) IDED | Dose protein (mg) |
| Q30 min | 0.5 |
| | 1 |
| | 1.5 |
| | 3 |
| | 6 |
| Cumulative | 12 |

TABLE 2
AR101 Bi-Weekly Up-Dosing Regimen Starting at Week 6

| Planned | Dupilumab/Placebo + AR101 Protein |
|---|---|
| Study Week | Dose protein (mg) |
| 6 | 6 |
| 7 | 6 |
| 8 | 12 |
| 9 | 12 |
| 10 | 20 |
| 11 | 20 |
| 12 | 40 |
| 13 | 40 |
| 14 | 80 |
| 15 | 80 |
| 16 | 120 |
| 17 | 120 |
| 18 | 160 |
| 19 | 160 |
| 20 | 200 |
| 21 | 200 |
| 22 | 240 |
| 23 | 240 |
| 24 | 300 |
| 25 | 300 |
| 26 | 300 |
| 27 | 300 |
| 28 (up to week 40) | DBPFC |

After 28 to 40 weeks of dupilumab treatment and 24 to 36 weeks of AR101, subjects who achieve 300 mg/day of AR101 for at least 2 weeks will undergo a DBPCFC to assess the level of peanut sensitivity.

Re-randomization at Week 28 to 40 for dupilumab group: Subjects in the dupilumab treatment group from the up-dosing phase will be re-randomized 1:1 placebo or dupilumab regardless of whether they achieve 300 mg/day AR101 for at least 2 weeks at visit 16 or discontinue. However, only those subjects who achieve 300 mg/day AR101 at visit 16 will be eligible to enter the maintenance phase. Subjects who do not achieve 300 mg/day AR101 for at least 2 weeks will enter a 12-week follow-up period.

Double-blind maintenance phase (for subjects who reach 300 mg/day AR101′): Subjects who achieve 300 mg/day AR101 for at least 2 weeks during the up-dosing period will be eligible to enter a 24-week maintenance phase in which all subjects will continue to receive AR101 300 mg/day at home. The 24-week maintenance duration will start from when the subject has their day 1 of their post up-dosing DBPCFC at visit 16. (Safety analysis of the maintenance period starts after the second day of the visit 16 post up-dosing DBPCFC.) Subjects will have a monthly clinic visit. If the on-site clinic visit cannot occur, phone visits can take place as long as there are no changes to the subject's AR101 dose. Subjects in the dupilumab treatment group will be randomly assigned to either continue dupilumab at the same dose as administered during the up-dosing period or to receive placebo. Subjects who received placebo during the up-dosing phase will continue to receive placebo in the maintenance phase. After 24 weeks of AR101 and 24 weeks of dupilumab or placebo, subjects will undergo a post maintenance DBPCFC to assess the level of peanut sensitivity at the end of the maintenance period.

Subjects who do not achieve 300 mg/day AR101 for at least 2 weeks at visit 16 and are not permitted to enter the maintenance phase, will enter a 12-week follow-up period (see below). The procedure for monitoring subjects for safety after in-clinic dosing is the same as for the up-dosing visits except that the initial period required for post-dose observation may be shortened to 30 minutes.

Double-blind, placebo-controlled food challenge: At visit 16 (the end of up-dosing) under intensive monitoring, all subjects who achieve 300 mg/day AR101 during the up-dosing phase for at least 2 weeks will undergo a post up-dosing DBPCFC up to 2044 mg (cumulative) peanut protein or placebo to assess desensitization. At visit 22 (the end of maintenance period), all subjects who maintain 300 mg/day AR101 will undergo a post maintenance DBPCFC up to 2044 mg (cumulative) peanut protein or placebo to assess desensitization. Dosing with AR101 should continue on the days between the 2 parts of the DBPCFC. The subject's sensitivity to peanut allergen is defined as the dose at which the subject experiences allergic reactions. All symptoms and signs will be evaluated and rated based on a standardized oral food challenge scoring system (CoFAR). Up-dosing during the DBPCFC will be stopped when the blinded assessor finds symptoms and/or signs that indicate a definite objective (Grade 1 [mild]) allergic reaction (CoFAR grading system) has occurred based on clinically significant changes in reported symptoms, physical findings, or vital signs that the subject is experiencing to the challenge material. Vital signs will be assessed every 15 to 30 minutes. In addition, subjects will be considered to have dose-limiting reactions if they experience any mild subjective reactions requiring pharmacologic intervention and/or any moderate/severe reaction. The DBPCFC will consist of 8 doses (peanut protein or placebo), given every 15 to 30 minutes: 1, 3, 10, 30, 100, 300, 600, 1000 mg resulting in a total challenge of up to 2044 mg (cumulative) peanut protein. Both peanut and oat protein will be concealed in a food that masks the taste. The food challenges will be performed on different days (1-day placebo [oat] protein, 1-day peanut protein, with order determined at random) at least 24 hours, but not more than 7 days, apart, and not within 24 hours of a dose of study drug. Subjects will be considered to have passed the DBPCFC if they do not experience any objective Grade 1 (mild) reaction by CoFAR grading system. If the subject experiences reactions, he/she will be treated with the necessary rescue medications. In addition, subjects will be considered to have passed the DBPCFC if they do not experience any mild subjective symptoms requiring pharmacological intervention and/or moderate or severe symptoms. He/she will be observed for a minimum of 2 hours after the final administered dose and discharged only when deemed clinically stable by a study physician. Symptom severity will be adjudicated by an independent, blinded assessor who is not involved in subject study visit conduct. When dosing elicits an acute reaction characterized by the appearance of only a mild subjective symptom or symptoms, the investigator will be required to assess whether the dose was or was not tolerated. The determination of tolerability must be made on the basis of clinical judgement. The following are presented as guidelines for determining whether a dose associated with the emergence of a mild subjective symptom or symptoms was tolerated. A dose eliciting only mild subjective symptoms may be considered to be tolerated if the symptoms are:
isolated to a single organ system except for airway (including tongue) or respiratory system;
resolve with no pharmaceutical intervention or with a single oral administration of an H1 antihistamine
do not require administration of epinephrine
are not worsening in intensity or distribution over time
resolve, or shows definite signs of resolving, in under 1 hour

On the day following DBPCFC, the site is to make telephone contact with the subject/subject's parent or guardian to inquire if any AEs (including allergic symptoms) occurred subsequent to the subject leaving the clinic, and to provide assistance in the recording of any such events.

Post-treatment follow-up period (12 weeks): All subjects will have a 12-week follow-up period after the end of treatment and will undergo safety, laboratory, and clinical assessments. The duration of the 12-week follow-up period is based on the time expected for drug levels to fall below the lower limit of quantification after the last dose of dupilumab. At the end of the 12-week follow-up period, subjects who passed a 444 mg (cumulative) peanut protein DBPCFC at visit 22 (the end of the maintenance period, week 52 to 64 will be eligible to undergo a final DBPCFC (up to 2044 mg cumulative), under intensive monitoring, at visit 25 (end of study, week 64 to 76) to assess the level of peanut sensitivity after 12 weeks off peanut and dupilumab to determine whether there is evidence of persistent effects and sustained unresponsiveness. Subjects who do not achieve 300 mg/day peanut protein at visit 16 (ideally week 28, and up to a maximum of week 40) will enter a 12 week follow-up period.

Missed doses of AR101: No attempt should be made to make up a missed dose if greater than 6 hours has elapsed from the usual time of dosing and the subject should continue with the next scheduled dose, which may be at home. If 2 consecutive doses are missed, the subject should continue with the next scheduled dose, which may be at home. If 3 to 7 consecutive doses are missed, the subject should return to the clinic for the next dose.

### Patient Selection

The target population includes male and female subjects ages 6 to 17 years inclusive with a history of peanut allergy confirmed by peanut SPT, peanut-specific IgE, and by the level of peanut protein safely ingested during a peanut DPBCFC.

Inclusion Criteria: A subject must meet the following criteria to be eligible for inclusion in the study: (1) age 6 to 17 years (inclusive); (2) subject has a clinical history of allergy to peanuts or peanut-containing foods (symptom[s] of reaction due to exposure); (3) experience dose-limiting symptoms at or before the 100 mg challenge dose (5144 mg cumulative) of peanut protein (measured as 200 mg of peanut flour) on screening DBPCFC conducted in accordance with PRACTALL (Practical Issues in Allergology, Joint United States/European Union Initiative) guidelines; and not experiencing dose-limiting symptoms to placebo; (4) serum IgE to peanut of ≥10 kUA/L and/or a SPT to peanut >8 mm compared to a negative control; (5) subjects/legal guardians must be trained on the proper use of the epinephrine autoinjector device to be allowed to enroll in the study; (6) subjects with other known food allergies must agree to eliminate these other food items from their diet so as not to confound the safety and efficacy data from the study; (7) willing and able to comply with all clinic visits and study-related procedures; (8) written informed consent from parent/guardian for minor subjects; (9) written assent from minor subjects as appropriate (e.g., above the age of 6 years or the applicable age per local regulatory requirements).

Exclusion Criteria: A subject who meets any of the following criteria will be excluded from the study: (1) any previous exposure to marketed dupilumab or dupilumab in a clinical trial; (2) member of the clinical site study team or his/her immediate family; (3) history of other chronic disease (other than asthma, AD, or allergic rhinitis) requiring therapy (e.g., heart disease, diabetes, hypertension) that, in the opinion of the principal investigator, would represent a risk to the subject's health or safety in this study or the subject's ability to comply with the study protocol; (4) history of frequent or recent severe, life-threatening episode of anaphylaxis or anaphylactic shock as defined by more than 3 episodes of anaphylaxis within the past year and/or an episode of anaphylaxis within 60 days of screening DBPCFC; (5) history of eosinophilic GI disease; (6) current participation or participation within 6 months prior to screening in any other interventional study; (7) asthma at time of enrollment with any of the following: (i) FEV1<80% of predicted, or ratio of FEV1 to forced vital capacity (FEV1/FVC) <75% of predicted with or without controller medications; or (ii) inhaled corticosteroids (ICS) dosing of >500 μg daily fluticasone (or equivalent ICS based on National Heart, Lung, and Blood Institute dosing chart); or (iii) one hospitalization in the past year for asthma; or (iv) emergency room visit for asthma within 6 months prior to screening; (8) use of systemic corticosteroids within 2 months prior to screening; (9) use of omalizumab within 6 months prior to screening; (10) use of other forms of allergen immunotherapy (e.g., oral, SC, patch, or sublingual) or immunomodulatory therapy (not including corticosteroids) within 3 months prior to screening; (11) use of antihistamines within 5 days prior to screening and within 5 days prior to SPTs and day 1 of DBPCFCs; (12) use of any agents known or likely to interact with epinephrine (e.g., beta-blockers, angiotensin converting enzyme-inhibitors, tri-cyclic antidepressants, or other drugs), within 3 weeks prior to screening; (13) allergy to oat (placebo in DBPCFC); (14) hypersensitivity to epinephrine and any of the excipients in the epinephrine product; (15) history of a mast cell disorder, including mastocytosis, urticarial pigmentosa, and hereditary or idiopathic angioedema; (16) treatment with a live (attenuated) vaccine within 3 months prior to screening and during the study. All subjects must have received all vaccinations per Advisory Committee on Immunization Practices (ACIP) or local guidelines for measles, mumps, rubella, and varicella at least 3 months prior to enrollment in the study. (17) active chronic or acute infection requiring systemic treatment with antibiotics, antivirals, antiparasitics, antiprotozoals, or antifungals within 2 weeks prior to the baseline visit. NOTE: subjects may be rescreened after the infection resolves. (18) history of malignancy within 5 years before the screening visit, except completely treated in situ carcinoma of the cervix, completely treated and resolved non-metastatic squamous or basal cell carcinoma of the skin; (19) established diagnosis of a primary immunodeficiency disorder (e.g., Severe Combined Immunodeficiency, Wiskott Aldrich Syndrome, DiGeorge Syndrome, X-linked Agammaglobulinemia, Common Variable Immunodeficiency), or secondary immunodeficiency; (20) known history of human immunodeficiency virus (HIV) infection or HIV seropositivity at the screening visit; (21) established diagnosis of hepatitis B viral infection at the time of screening or is positive for hepatitis B surface antigen (HBsAg) or hepatitis B core antibody (HBcAb) at the time of screening; (22) body weight ≤17 kg; (23) pregnant or breastfeeding women, women planning to become pregnant or breastfeed during the study; (24) girls at or beyond menarche who are not sexually abstinent and are unwilling to practice highly effective contraception prior to the initial dose/start of the first treatment, during the study, and for at least 12 weeks after the last dose.

### Study Treatments

Dupilumab 150 mg/mL: Each 2.25 mL single-use, prefilled glass syringe with snap-off cap delivers 300 mg of study drug (2.0 mL of a 150 mg/mL solution).

Dupilumab 175 mg/mL: Each 1.14 mL single-use, prefilled glass syringe with snap-off cap delivers 200 mg of study drug (1.14 mL of a 175 mg/mL solution).

Dupilumab 150 mg/ml: Each 0.67 mL single-use, prefilled glass syringe with snap-off cap delivers 100 mg study drug (0.67 mL of a 150 mg/mL solution).

Placebo matching dupilumab is prepared in the same formulation without the addition of protein (i.e., active substance, anti-IL-4Rα monoclonal antibody). Three matching placebo formulations will be used: (1) 2 mL placebo matching 300 mg dupilumab formulation; (2) 1.14 mL placebo matching 200 mg dupilumab formulation; (3) 0.67 mL placebo matching 100 mg dupilumab formulation.

Subcutaneous injection sites of the study drug should be alternated among the different quadrants of the abdomen (avoiding navel and waist areas), upper thighs, and upper arms so that the same site is not injected for 2 consecutive administrations.

AR101 for oral immunotherapy and IDED: AR101 (PALFORZIA; Aimmune Therapeutics, Inc.) is characterized peanut allergen in the form of peanut flour, formulated with a bulking agent (maize starch, microcrystalline cellulose, and other excipients to prevent clumping) and a flow agent in premeasured graduated doses. The AR101 drug product is encapsulated in hydroxypropyl methylcellulose (HPMC) capsules. The capsules used in the Initial Escalation Period and Up-dosing Period of this study currently include the following strengths: 0.5, 1, 10, 20, and 100 mg each of peanut protein. AR101 is characterized by high performance liquid chromatography and by specific enzyme-linked immunosorbent assay for key allergenic proteins to demonstrate stability and lot-to-lot consistency.
AR101 0.5 mg: each opaque white HPMC capsule delivers 0.5 mg peanut protein
AR101 1 mg: each opaque red HPMC capsule (with white bars) delivers 1 mg peanut protein
AR101 10 mg: each opaque blue HPMC capsule delivers 10 mg peanut protein
AR101 20 mg: each opaque white HPMC capsule (with grey bars) delivers 20 mg peanut protein
AR101 100 mg: each opaque Swedish orange HPMC capsule (with grey and black bars) delivers 100 mg peanut protein

AR101 capsules will be provided in prepackaged thermoform blister wallet cards with features to allow access to each dose in dosing kits. Appropriate combinations of capsules are used to provide the required AR101 doses (e.g., one 20 mg capsule and one 100 mg capsule to provide a 120 mg dose). For the escalation periods, each individual kit will contain 21 daily doses at a given dose level, enough to supply 2 weeks of dosing plus 7-day coverage to accommodate potential visit scheduling issues. For the maintenance period dosing, a 300 mg AR101 dose will be provided in sealed, foil-laminate sachets (1 sachet/day). Sachets will be provided in kits containing 35 individual doses. AR101 will be stored in a secure location at each study site and kept refrigerated between 2° C. and 8° C.

After 4 full weeks of either dupilumab or placebo treatment, subjects start using a standardized regimen of single IDED AR101 (0.5 mg to a maximum of 6 mg, 12 mg cumulative) over approximately 5 to 6 hours. Subjects will begin a 3 mg/day oral dose at home for the next 14 days followed by bi-weekly up-dosing to a maximum of 300 mg/day AR101 at home.

Peanut for food challenges: For the DBPCFC, peanut flour mixture (containing ˜50% peanut protein) or a placebo (oat) flour mixture, mixed in a vehicle food, will be used. The placebo flour mixture will be supplied pre-mixed with a small amount of artificial peanut flavor to provide a reasonable degree of taste-matching of the final placebo/vehicle food mixture to the peanut/vehicle food mixture. Investigational sites will be provided with standardized recipes for preparation of the DBPCFC in a separate manual of procedures.

### Rescue Treatments

The following concomitant treatments will require permanent study drug discontinuation: (1) treatment with an investigational drug (other than dupilumab); (2) treatment with immunomodulating biologic agents, including anti-IgE and anti-IL-5; (3) treatment with allergen immunotherapy other than AR101; (4) treatment with systemic (oral, IV, IM, SC) corticosteroids for a duration of more than 5 continuous days, more than 15 days in total, or within 2 days prior to DBPCFCs.

The following concomitant treatments of allergic reactions will NOT require permanent study drug discontinuation: (1) IM or SC administration of epinephrine; (2) oral antihistamines; (3) short acting inhaled bronchodilators; (4) inhaled corticosteroids; (5) systemic (oral, IV, IM, SC) corticosteroids for a duration of less than 5 continuous days, less than 15 days in total, and at least 2 days prior to DBPCFCs.

### Procedures and Assessments

A variety of parameters will be collected during the study to assess the efficacy, safety, pharmacokinetics, and pharmacodynamics of treatment with dupilumab and AR101 OIT.

### Efficacy Parameters

Double-Blind Placebo-Controlled Food Challenge: The subject's sensitivity to peanut allergen is defined as the dose at which the subject experiences allergic reactions. All symptoms and signs will be evaluated and rated based on a standardized oral food challenge scoring system. Up-dosing during the DBPCFC will be stopped when the principal investigator (or designee) finds symptoms and/or signs that indicate a definite objective allergic reaction (CoFAR grading system [see Table 4]) has occurred based on clinically significant changes in reported symptoms, physical findings, or vital signs that the subject is experiencing to the challenge material. The challenge will consist of 8 doses (peanut protein or placebo), given every 15 to 30 minutes: 1, 3, 10, 30, 100, 300, 600, 1000 mg, up to 2044 mg peanut protein (cumulative). See Table 3. Both peanut and oat protein will be concealed in a food that masks the taste. The food challenges will be performed on different days (1-day placebo [oat] protein, 1-day peanut protein, with order determined at random) at least 24 hours, but not more than 7 days apart, and not within 24 hours of a dose of study drug. After the last dose of the DBPCFC, the subject will be monitored for at least 2 hours and then discharged from the clinic. Subjects will be considered to have tolerated the DBPCFC and passed if they do not experience any objective Grade 1 (mild) reaction by CoFAR grading system. If the subject experiences reactions, they will be treated with the necessary rescue medications. Symptom severity will be adjudicated by an independent, blinded assessor who is not involved in performing the baseline food challenge.

TABLE 3
Peanut DBPCFC Schedule of Dosing Performed at Screening
and Visits 16, 22, and 25

| | Challenge Doses | | | |
|---|---|---|---|---|
| | Amount of Peanut | Amount of Peanut | Cumulative | Cumulative Amount of |
| | Protein at | Flour with | Amount of Peanut | Peanut Protein (mg) |
| | Each Challenge | 50% Protein | Protein (mg) at | post-screening (V16, |
| | Dose (mg) | Content (mg) | Screening | V22, and V25) |
| Screening | 1 | 2 | 1 | 1 |
| Screening | 3 | 6 | 4 | 4 |
| Screening | 10 | 20 | 14 | 14 |
| Screening | 30 | 60 | 44 | 44 |
| Screening | 100 | 200 | 144 | 144 |
| Endpoint | 300 | 600 | — | 444 |
| Endpoint | 600 | 1200 | — | 1044 |
| Endpoint | 1000 | 2000 | — | 2044 |

TABLE 4
Allergic Reaction Severity Grading

| Grade 1- | Grade 2- | Grade 3- | Grade 4- | Grade 5- |
|---|---|---|---|---|
| Mild | Moderate | Severe | Life Threatening | Death |
| Transient or mild | Symptoms that produce mild | Marked limitation in activity, | Extreme limitation in activity, | Death |
| discomforts (<48 hrs), no or | to moderate limitation in | some assistance usually | significant assistance required; | |
| minimal medical | activity, some assistance may | required; medical | significant medical | |
| intervention/therapy | be needed; no or minimal | intervention/therapy required, | intervention/therapy required. | |
| required. These symptoms | intervention/therapy is | hospitalization is possible. | Hospitalization is probable. | |
| may include pruritus, | required. Hospitalization if | Symptoms may include | Symptoms may include | |
| swelling or rash, abdominal | possible. These symptoms may | bronchospasm with dyspnea, | persistent hypotension and/or | |
| discomfort or other | include persistent hives, | severe abdominal pain, throat | hypoxia with resultant | |
| transient symptoms. | wheezing without dyspnea, | tightness with hoarseness, | decreased level of | |
| | abdominal | transient hypotension among | consciousness and/or | |
| | discomfort/increased vomiting | others. Parenteral | incontinence or other life- | |
| | or other symptoms | medication(s) are usually | threatening symptoms. | |
| | | indicated. | | |

Fractional Exhaled Nitric Oxide (FeNO): Fractional exhaled nitric oxide is a non-invasive marker that has been shown to correlate with allergic airway inflammation and IgE sensitization. The measurement of FeNO has shown predictive value on the outcome of peanut oral food challenge (Preece, 2014). Although no manufacturer has validated the FeNO device for children under 7 years old, due to inadequate exhalation force, there are no contraindications with the use of FeNO for 6-year-old children. Also, specific guidance and reference ranges are available for collection in children as young as 6 years of age (Brody, 2013) (Menou, 2017). As FeNO is an exploratory biomarker, it will be collected for children 6 years old unless the investigator declines to collect. Fractional exhaled nitric oxide will be performed prior to spirometry.

Peanut Skin Prick Test (SPT): The standard SPT is performed on the volar surface of the subject's forearm using standard whole peanut extract reagent, 1:10 w/v (ALK-Abello) and will only be performed at screening. A positive result is ≥3 mm determined by averaging maximal perpendicular wheal diameters 15 minutes after applying the lancet. The positive control is histamine base, 6 mg/mL (ALK-Abello) and with a wheal >3 mm indicating a valid test. The negative control is glycerol saline.

The titrated SPT is the skin testing for atopic response at different concentrations of peanut extract with saline as negative control and histamine as positive controls. The test will be performed at specified time points 4. Testing of peanut extract will be conducted at the following dilutions: Neat, 1:20, 1:200, 1:2000, 1:20,000. The longest diameter and longest orthogonal diameter will be collected.

Mean wheal size induced by peanut extract, histamine, and saline at each concentration will be calculated by adding the longest diameter to the longest orthogonal diameter and dividing by 2. Normalized SPT will be calculated by subtracting mean saline wheal size from mean peanut wheal size.

Eczema Area and Severity Index: The EASI is a validated measure used in clinical practice and clinical trials to assess the severity and extent of atopic dermatitis (AD) (Hanifin, 2001). The EASI is a composite index with scores ranging from 0 to 72. Four AD disease characteristics (erythema, thickness [induration, papulation, edema], scratching [excoriation], and lichenification) will each be assessed for severity by the investigator or designee on a scale of “0” (absent) through “3” (severe). In addition, the area of AD involvement will be assessed as a percentage by body area of head, trunk, upper limbs, and lower limbs, and converted to a score of 0 to 6. In each body region, the area is expressed as 0, 1 (1% to 9%), 2 (10% to 29%), 3 (30% to 49%), 4 (50% to 69%), 5 (70% to 89%), or 6 (90% to 100%). The EASI will be collected for subjects with AD at screening and at subsequent specified time points.

Asthma Control Questionnaire: The Asthma Control Questionnaire (ACQ) is a validated questionnaire to evaluate asthma control. The questionnaire will be administered only to the subset of subjects with a medical history of asthma and who fluently speak a language in which the questionnaire is presented (based on availability of validated translations in participating countries), at specified time points. The ACQ-5 is for subjects aged 11 years or more and the ACQ-interviewer administered (IA) is for subjects aged 6 to 10 years. The ACQ has been fully validated for all children 6-17 years when the self-administered adult version is used by children 11 years and older and the interviewer-administered version is used for children 6-10 years. Subjects will continue using the ACQ version first administered at screening regardless of moving to the next age bracket.

Daily Allergy Symptom Diary: The daily allergy symptom diary is a questionnaire that was designed to capture the daily signs and symptoms of peanut OIT. The measure was developed with input from patients currently or recently treated with peanut OIT as well as clinicians with experience treating patients with peanut OIT. The daily allergy symptom diary has also been cognitively debriefed within the target population. In addition to daily allergy symptoms, the e-diary will also collect data on dupilumab injections, peanut OIT dosing, use of medications to treat symptoms of peanut OIT, and health states.

Food Allergy Quality of Life Questionnaire: The Food Allergy Quality of Life Questionnaire (FAQLQ) is a validated food allergy-specific health-related quality of life (HRQL) questionnaire, which measures the impact of social and dietary limitations and assesses the emotional impact of these restrictions on the lives of patients. Patients self-report the impact of food allergy on HRQL using different forms of FAQLQ depending on their age; the child form (FAQLQ-CF) is used by patients aged 8 to 12 years and the teenager form (FAQLQ-TF) is used for patients aged 13 to 17. The parent form (FAQLQ-PF) is a measure of children's HRQL that is reported by parent proxy from the child's perspective and can be used for patients of ages 0 to 12 years. The FAQLQ will be administered to subject and, when appropriate, parents at specified time points. Subjects will continue using the FAQLQ version first administered at baseline regardless of moving to the next age bracket.

### Safety Procedures

Vital signs, including temperature, sitting blood pressure, pulse, and respiration rate will be collected pre-dose at specified time points. During the DBPCFC, IDE and up-dosing, vital signs will be monitored every 15 to 30 minutes. For post-dose monitoring, only pulse and blood pressure need to be taken as part of safety monitoring.

A thorough and complete physical examination will be performed at specified time points. Care should be taken to examine and assess any abnormalities that may be present, as indicated by the subject's medical history.

A spirometer that meets the 2005 American Thoracic Society/European Respiratory Society recommendations will be used to measure FEV1 and/or PEF. During DBPCFC, spirometry should be performed before and after the challenge. The same spirometer and standard spirometric techniques, including calibration, should be used to perform spirometry at different visits and, whenever possible, the same person should perform the measurements (Pellegrino, 2005). FeNO should be done prior to spirometry.

Hematology, chemistry, urinalysis, and pregnancy testing samples will be analyzed by a central laboratory. Samples for laboratory testing will be collected at specified visits. Tests will include blood chemistry, hematology, and urinalysis tests.

### Pharmacokinetic and Biomarker Procedures

Biomarker samples will be collected at specified time points. Biomarker measurements will be performed in serum or plasma to determine effects on biomarkers of relevant physiological and pathogenic processes. The biomarkers studied will be ones believed to be relevant to the pathophysiology of peanut allergy, mechanism of action of dupilumab, and possible toxicities. Biomarkers studied may include but need not be limited to: Total IgE, Thymus and Activation-Regulated Chemokine, specific IgE, IgG4, and IgG against peanut extract and main peanut allergen components (including but not limited to Ara h1, Ara h2, and Ara h3), blood stimulation in TruCulture (supernatant cytokine and chemokine profiling), basophil sensitivity to allergen stimulation and peanut-specific T-cell profiling.

Research samples (serum/plasma/peripheral blood mononuclear cells) will be banked for future biomedical research related to Type 2/Th2 inflammation, allergic diseases, IL-4/IL-13, dupilumab (including mechanism of action, efficacy, toxicity), and circulating factors that inhibit allergen-specific IgE from binding to allergen (e.g., IgE blocking factor). The research may also include, but is not limited to, the study of allergen epitope diversity recognized by allergen-specific antibodies (both IgE and IgG4), T-cell and B-cell receptor repertoire (requires DNA/RNA sequencing), and the effects of dupilumab treatment on non-peanut allergies.

### Statistical Methods

For continuous variables, descriptive statistics will include the following information: the number of subjects reflected in the calculation (n), mean, median, first quartile (Q1), third quartile (Q3), standard deviation, minimum, and maximum. For categorical or ordinal data, frequencies and percentages will be displayed for each category. All data will be summarized by treatment groups as outlined below:
Double-blind treatment period with pre-dosing and up-dosing of AR101 (28 to 40 weeks): placebo plus AR101 vs dupilumab plus AR101.
Double-blind maintenance phase (24 weeks): continuously on placebo+AR101, previously on dupilumab+AR101 and re-randomized to placebo+AR101, continuously on dupilumab+AR101.

The primary endpoint will be analyzed using the Cochran-Mantel-Haenszel test adjusted by randomization stratification factors to assess the treatment difference in the proportion of responders (i.e., those who “pass” a post up-dosing DBPCFC with 2044 mg [cumulative] peanut protein at visit 16 [week 28 with a visit window of −7/+30 days]) in the modified full analysis set (mFAS). The mFAS includes all FAS subjects who undergo bio-weekly in-clinic up-dosing as specified in the protocol and undergo the post up-dosing DBPCFC at week 28 (visit 16 with a window of −7/+30 days) or discontinue from study prior to week 28 (visit 16 with a window of −7/+30 days). Estimate of treatment difference, p-value, and the 2-sided 95% confidence interval will be provided. In addition, all efficacy endpoints will be evaluated on the full analysis set (FAS) as a supportive analysis. The full analysis set (FAS) includes all randomized subjects including at sites impacted by COVID-19. In a protocol amendment made to accommodate COVID-19 site restrictions, double-blind treatment period was extended from 28 weeks to up to 40 weeks. FAS subjects who attended optional visits between week 28 and week 40 will have increased duration of exposure to AR101 and dupilumab/placebo. If a subject does not have available post up-dosing DBPCFC data at visit 16, the subject will be considered as a non-responder regardless of reasons for missing data.

Results from Interim Analysis at the End of the Up-Dosing Period

### Patient Baseline Characteristics

An interim analysis was performed at the end of the up-dosing period. The baseline demographics and disease characteristics for the FAS and mFAS patient populations are shown in Table 5. The baseline biomarker parameters are shown in Table 6. As shown in Table 5, a high percentage of patients had a concomitant atopic disease (atopic dermatitis, asthma, or both atopic dermatitis and asthma). A majority of patients also had a history of multiple food allergies. A majority of patients also had a history of peanut anaphylaxis.

TABLE 5
Baseline Demographics and Disease Characteristics

| | FAS | | | mFAS | | |
|---|---|---|---|---|---|---|
| | Placebo + | Dupilumab + | | Placebo + | Dupilumab + | |
| | AR101 | AR101 | Total | AR101 | AR101 | Total |
| Parameters | (N = 50) | (N = 98) | (N = 148) | (N = 39) | (N = 84) | (N = 123) |
| Male, n (%) | 32 (64.0%) | 60 (61.2%) | 92 (62.2%) | 25 (64.1%) | 51 (60.7%) | 76 (61.8%) |
| Age, mean (SD) | 10.9 (3.00) | 11.3 (3.12) | 11.1 (3.08) | 11.0 (3.17) | 11.3 (3.17) | 11.2 (3.16) |
| ≥6 and <12 y/o, n (%) | 30 (60.0%) | 52 (53.1%) | 82 (55.4%) | 23 (59.0%) | 43 (51.2%) | 66 (53.7%) |
| ≥12 and ≤17 y/o, n (%) | 20 (40.0%) | 46 (46.9%) | 66 (44.6%) | 16 (41.0%) | 41 (48.8%) | 57 (46.3%) |
| Weight (kg), mean (SD) | 41.1 (17.39) | 43.4 (17.67) | 42.6 (17.55) | 41.9 (18.69) | 44.0 (18.38) | 43.3 (18.43) |
| <30 kg, n (%) | 16 (32.0%) | 28 (28.6%) | 44 (29.7%) | 13 (33.3%) | 24 (28.6%) | 37 (30.1%) |
| ≥30 kg and <60 kg, n (%) | 26 (52.0%) | 56 (57.1%) | 82 (55.4%) | 19 (48.7%) | 47 (56.0%) | 66 (53.7%) |
| ≥60 kg, n (%) | 8 (16.0%) | 14 (14.3%) | 22 (14.9%) | 7 (17.9%) | 13 (15.5%) | 20 (16.3%) |
| BMI, mean (SD) | 18.1 (3.90) | 18.9 (4.13) | 18.7 (4.06) | 18.3 (4.19) | 19.1 (4.35) | 18.9 (4.31) |
| Duration of peanut allergy | 9.8 (3.54) | 10.7 (3.66) | 10.4 (3.64) | 9.8 (3.84) | 10.7 (3.81) | 10.5 (3.83) |
| diagnosis (yrs), mean (SD) | | | | | | |
| Age at peanut allergy onset | 2.0 (2.11) | 1.3 (1.62) | 1.6 (1.82) | 2.1 (2.31) | 1.3 (1.70) | 1.6 (1.93) |
| (y/o), mean (SD) | | | | | | |
| <5, n (%) | 43 (86.0%) | 90 (91.8%) | 133 (89.9%) | 33 (84.6%) | 76 (90.5%) | 109 (88.6%) |
| ≥5 and <10, n (%) | 6 (12.0%) | 8 (8.2%) | 14 (9.5%) | 5 (12.8%) | 8 (9.5%) | 13 (10.6%) |
| ≥10 and ≤17, n (%) | 1 (2.0%) | 0 | 1 (0.7%) | 1 (2.6%) | 0 | 1 (0.8%) |
| Co-morbid of both Atopic | 22 (44.0%) | 44 (44.9%) | 66 (44.6%) | 19 (48.7%) | 37 (44.0%) | 56 (45.5%) |
| Dermatitis and Asthma | | | | | | |
| disease, n (%) | | | | | | |
| Had Atopic Dermatitis | 31 (62.0%) | 69 (70.4%) | 100 (67.6%) | 27 (69.2%) | 60 (71.4%) | 87 (70.7%) |
| history, n (%) | | | | | | |
| Had Asthma history, n (%) | 33 (66.0%) | 58 (59.2%) | 91 (61.5%) | 26 (66.7%) | 48 (57.1%) | 74 (60.2%) |

TABLE 6
Baseline Biomarker Parameters

| | FAS | | | mFAS | | |
|---|---|---|---|---|---|---|
| | Placebo + | Dupilumab + | | Placebo + | Dupilumab + | |
| | AR101 | AR101 | Total | AR101 | AR101 | Total |
| Parameters | (N = 50) | (N = 98) | (N = 148) | (N = 39) | (N = 84) | (N = 123) |
| Screening SPT (mm), | 11.3 | 12.0 | 12.0 | 12.1 | 12.7 | 12.5 |
| Median (IQR) | (9.0:15.3) | (10.0:15.0) | (10.0:15.3) | (4.03) | (3.83) | (3.89) |
| Screening sIgE (kUA/L) from | | | | | | |
| IVRS | | | | | | |
| ≤100, n (%) | 28 (56.0%) | 58 (59.2%) | 86 (58.1%) | 20 (51.3%) | 51 (60.7%) | 71 (57.7%) |
| >100, n (%) | 22 (44.0%) | 40 (40.8%) | 62 (41.9%) | 19 (48.7%) | 33 (39.3%) | 52 (42.3%) |
| Screening IgE (kUA/L) from | 71.1 | 56.1 | 61.7 | 67.5 | 56.1 | 56.8 |
| EDC, Median (IQR) | (32.1:196.0) | (15.1:185.0) | (18.1:193.0) | (18.3:271) | (12.2:182.5) | (14.5:198) |
| 0.35-<17.5 | 8 (16.0%) | 24 (24.5%) | 32 (21.6%) | 8 (20.5%) | 23 (27.4%) | 31 (25.2%) |
| 17.5-<52.5 | 8 (16.0%) | 19 (19.4%) | 27 (18.2%) | 7 (17.9%) | 14 (16.7%) | 21 (17.1%) |
| 52.5-≤100 | 11 (22.0%) | 18 (18.4%) | 29 (19.6%) | 5 (12.8%) | 16 (19.0%) | 21 (17.1%) |
| >100 | 23 (46.0%) | 37 (37.8%) | 60 (40.5%) | 19 (48.7%) | 31 (36.9%) | 50 (40.7%) |
| ≥200 | 10 (20.0%) | 19 (19.4%) | 29 (19.6%) | 10 (25.6%) | 16 (19.0%) | 26 (21.1%) |
| Screening sIgG4 (ug/mL), | 0.5 | 0.5 | 0.5 | 0.4 | 0.5 | 0.5 |
| Median (IQR) | (0.2:1.1) | (0.3:1/1) | (0.3:1.1) | (0.2:1.2) | (0.3:1.1) | (0.3:1.1) |
| Screening total IgE (IU/mL), | 473.5 | 505.0 | 495.5 | 502.0 | 547.5 | 521.0 |
| Median (IQR) | (290.0:885.0) | (287:1048.0) | (288.0:914.0) | (274:911) | (299.0:1066.0) | (293:947) |

Table 7 shows the analysis populations for this study. The modified full analysis set (mFAS) was considered as the primary analysis set for all efficacy endpoints. The full analysis set (FAS) was evaluated for all efficacy endpoints as supportive analyses.

TABLE 7
Patient Disposition for mFAS and FAS Analysis Populations

| | mFAS (N = 123) | | | FAS (N = 148) | | |
|---|---|---|---|---|---|---|
| | Placebo + | Dupilumab + | | Placebo + | Dupilumab + | |
| | AR101 | AR101 | Total | AR101 | AR101 | Total |
| N | 39 | 84 | 123 | 50 | 98 | 148 |
| Completed up-dosing | 29 | 75 | 104 | 38 | 84 | 122 |
| V16/Week 28 | (74.4%) | (89.3%) | (84.6%) | (76%) | (85.7%) | (82.4%) |
| (a window of −7/+30) | | | | | | |
| Discontinued with | 10 | 9 | 19 | 11 | 10 | 21 |
| reasons | (25.6%) | (10.7%) | (15.4%) | (22%) | (10.2%) | (14.2%) |
| Due to COVID-19 | 1 | 2 | 3 | 2 | 2 | 4 |
| | (2.6%) | (2.4%) | (2.4%) | (4%) | (2.04%) | (2.7%) |
| Not due to COVID-19 | 9 | 7 | 16 | 9 | 8 | 17 |
| (AE, lost FU, etc.) | (23.1%) | (8.3%) | (13.0%) | (18.0%) | (8.2%) | (11.5%) |
| Ongoing undergo up- | 0 | 0 | 0 | 1 | (4.1%) | 5 |
| dosing DBPCFC | | | | (2%) | | (3.4%) |

### Efficacy

The key efficacy results are shown in Table 8 (mFAS population) and Table 9 (FAS population). The primary endpoint was the proportion of subjects who are able to pass a DBPCFC with 2044 mg (cumulative) peanut protein at visit 16. For both the mFAS and FAS populations, a higher proportion of patients treated with dupilumab+AR101 were able to pass the DBPCFC versus patients treated with AR101 alone (mFAS: 56.0% versus 35.9%, treatment effect versus placebo of 20.2%; FAS: 54.1% versus 34.0%, treatment effect versus placebo of 20.3%).

Treatment with dupilumab+AR101 increased the proportion of patients who were able to reach a 300 mg/day dose of AR101 for at least two weeks by visit 16 (mFAS: 89.3% versus 76.9% for AR101 alone; FAS: 89.7% versus 81.6% for AR101 alone). Treatment with dupilumab+AR101 also increased the amount of cumulative tolerated dose of peanut protein during a DBPCFC from baseline to visit 16, as measured by raw dose or log transformed dose. See, Tables 8 and 9.

TABLE 8
Summary of Key Efficacy Results (mFAS)

| | | PBO + AR101 | Dupi + AR101 | TRT Effect vs PBO |
|---|---|---|---|---|
| Category | Endpoints | (N = 39) | (N = 84) | (95% CI) [pvalue] |
| Primary | Proportion of subjects who “pass” a DBPCFC | 14 (35.9%) | 47(56.0%) | 20.2% (1.27%, 39.19%) |
| | with 2044 mg (cumulative) peanut protein at visit | | | [0.0420] |
| | 16 [1], n (%) | | | |
| Secondary | Change in cumulative tolerated dose in log | Baseline: 1.99 (1.26) | Baseline: 2.39 (1.42) | 0.67 (0.031, 1.305) |
| | transformed of peanut protein during a DBPCFC | Visit 16: 6.00 (1.89) | Visit 16: 6.69 (1.47) | [0.0400] |
| | from baseline to visit 16 [2], mean SD) | Change, LS mean (se): | Change, LS mean (se): | |
| | | 3.75 (0.249) | 4.45 (0.21) | |
| | Change in cumulative tolerated dose (raw dose) | Baseline: 14.0 (15.1) | Baseline: 21.4 (18.1) | 333.6 (23.8, 643.4) |
| | of peanut protein during a DBPCFC from | Visit 16: 1014.7 (828.3) | Visit 16: 1355.67 (800) | [0.035] |
| | baseline to visit 16 [2], mean (SD) | Change: 1000.7 (829.8) | Change: 1334.3 (797.1) | |
| | Proportion of subjects who reach the 300 mg/day | 30 (76.9%) | 75 (89.3%) | 11.9 (−3.61%, 27.44%) |
| | dose of AR101 for at least 2 weeks by visit 16 | | | [0.0806] |
| | [3], n (%) | | | |
| | Time (Days) from randomization to the first time | 170.0 (169.0, 183.0) | 170.0 (170.0, 171.0) | HR: 1.164 (0.743- |
| | when subjects reach the 300 mg/day dose of | | | 1.825) |
| | AR101 during the treatment phase (up to visit | | | [0.5074] |
| | 16), median time (95% CI) | | | |

[1] MI/Non-responder for binary data: The multiple imputation (MI) method to impute the missing values due to COVID-19 and non-responder to missing values due to other reasons
[2] Mean baseline cumulative does to impute all missing values regardless any reason
[3] On-going subjects were excluded from analysis. No ongoing subject in mFAS.

TABLE 9
Summary of Key Efficacy Results (FAS)

| | | PBO + AR101 | Dupi + AR101 | TRT Effect vs PBO |
|---|---|---|---|---|
| Category | Endpoints | (N = 50) | (N = 98) | (95% CI) [pvalue] |
| Primary | Proportion of subjects who “pass” a DBPCFC | 17 (34.00%) | 53 (54.08%) | 20.26% (3.30, 37.18) |
| | with 2044 mg (cumulative) peanut protein at visit | | | [0.0223] |
| | 16 [1], n (%) | | | |
| | Change in cumulative tolerated dose in log | Baseline: 2.17 (1.28) | Baseline: 2.46 (1.42) | 0.53 (−0.05, 1.11) |
| | transformed of peanut protein during a DBPCFC | Visit 16: 6.03 (1.81) | Visit 16: 6.56 (1.58) | [0.0744] |
| | from baseline to visit 16 [2], mean SD) | Change, LS mean (se): | Change, LS mean (se): | ~1.1 folds |
| | | 3.75 (0.249) | 4.27 (0.194) | |
| | | 1.6 folds | 2.67 folds | |
| | Change in cumulative tolerated dose (raw dose) | Baseline: 16.5 (16.4) | Baseline: 22.5 (18.5) | 282.9 (1.4, 564.4) |
| Secondary | of peanut protein during a DBPCFC from | Visit 16: 998.6 (815.5) | Visit 16: 1304.0 (820.0) | [0.0489] |
| | baseline to visit 16 [2], mean (SD) | Change: 982.0 (815.6) | Change: 1281.5 (818.6) | ~1.3 folds |
| | | 60.5 folds | ~58 folds | |
| | Proportion of subjects who reach the 300 mg/day | 40/49 (81.63%) | 87/97 (89.69%) | 8.34% (−4.272%, |
| | dose of AR101 for at least 2 weeks by visit 16 | | | 20.961%) |
| | [3], n (%) | | | [0.1527] |
| | Time (Days) from randomization to the first time | 182.0 (169.0, 191.0) | 171.0 (171.0, 175.0) | HR: 1.420 (0.953- |
| | when subjects reach the 300 mg/day dose of | | | 2.117) |
| | AR101 during the treatment phase (up to visit | | | [0.0847] |
| | 16), median time (95% CI) | | | |

[1] MI/Non-responder for binary data: The multiple imputation (MI) method to impute the missing values due to COVID-19 and non-responder to missing values due to other reasons
[2] Mean baseline cumulative does to impute all missing values regardless any reason
[3] On-going subjects were excluded from analysis. No ongoing subject in mFAS.

### Tolerability and Safety

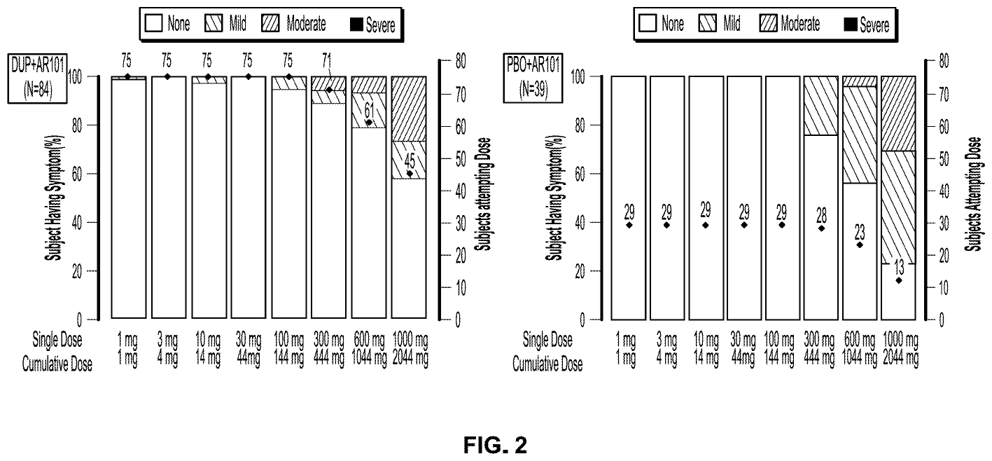

Patients were asked to use an e-diary to capture the daily signs and symptoms of allergies. As shown in Table 10 below, the percentage of days of daily e-diary allergic symptoms reported during up-dosing (excluding DBPCFCs) decreased in the dupilumab+AR101 group as compared with the placebo+AR101 group. Additionally, as shown in FIG. 2, during the Visit 16 DBPCFC the frequency and severity of allergic reactions was decreased in the dupilumab+AR101 group as compared with the placebo+AR101 group.

TABLE 10
Percentage of Days of Daily eDiary Allergic Symptoms Reported During Up-Dosing

| | % of days during up- | % of days | % of days during | % of days during up- |
|---|---|---|---|---|
| | dosing with any | during up-dosing | up-dosing with an | dosing with any |
| | peanut allergic | with a GI | itching allergic | severe (>7 of 10) |
| | symptoms | allergic symptom | symptom | allergic symptom |
| PBO + AR101 | 16.2 (20.3) | 11.3 (18.0) | 7.5 (11.9) | 3.0 (6.6) |
| (mean) (SD) % | | | | |
| PBO + AR101 | 6 | 3.2 | 3.2 | 1.2 |
| (median) (SD) % | | | | |
| DUP + AR101 | 10.6 (18.9) | 3.6 (5.8) | 7.4 (16.3) | 1.5 (2.4) |
| (mean) (SD) % | | | | |
| DUP + AR101 | 3.6 | 1.9 | 1.8 | 0.6 |
| (median) (SD) % | | | | |

Treatment with dupilumab was generally well tolerated and demonstrated an acceptable safety profile during the placebo-controlled peanut protein pre-dosing and up-dosing treatment periods. No new safety signals emerged in this population of peanut allergic children and adolescents with biweekly weight tiered dosing. No deaths occurred during the study. The percentage of subjects with at least one treatment-emergent adverse event (TEAE) during the 24-week up-dosing treatment period was similar between the placebo+AR101 group (76.0%) and the dupilumab+AR101 group (62.2%) (See Table 11). In 5 subjects TEAEs led to permanent discontinuation of study drug in the up-dosing period (2 in the placebo+AR101 group and 3 in the dupilumab+AR101 group. During the up-dosing period, injection site reactions were less common in the dupilumab+AR101 group (5.1%) than in the placebo+AR101 group (6.0%); all injection site reasons were assessed as mild or moderate in intensity.

The percentage of subjects reporting TEAE of anaphylactic reactions (adjudicated) during pre-dosing and up-dosing periods, excluding during Visit 16 DBPCFCs, was similar in the placebo+AR101 group (4.0%) and dupilumab+AR1010 group (5.1%) (Table 11). Epinephrine use for treatment of acute-allergic reactions during the study is summarized in Table 12.

TABLE 11
Overall Summary of TEAEs During Pre-Dosing and Up-Dosing Periods

| | Pre-dosing Period | | Up-dosing Period | |
|---|---|---|---|---|
| | Placebo | Dupilumab | Placebo + AR101 | Dupilumab + AR101 |
| % of Subjects with | (N = 50) | (N = 98) | (N = 50) | (N = 98) |
| Death | 0 | 0 | 0 | 0 |
| TEAE | 13 (26.0%) | 37 (37.8%) | 38 (76.0%) | 61 (62.2%) |
| SAE | 0 | 0 | 0 | 0 |
| Withdrawal from Treatment due to AE | 0 | 0 | 2 (4.0%) | 3 (3.1%) |
| TEAE of Special Interest (Adjudicated) | | | | |
| Anaphylactic reactions | 0 | 0 | 2 (4.0%) | 5 (5.1%) |
| Systemic or severe hypersensitivity reactions | 2 (4.0%) | 5 (5.1%) | 4 (8.0%) | 16 (16.3%) |
| TEAE of Anaphylactic Reactions (Adjudicated) at | Combined to | | 0 | 0 |
| V16 DBPCFCs | Up-Dosing Period | | | |
| TEAE of Anaphylactic Reactions (Adjudicated) | | | 2 (4.0%) | 5 (5.1%) |
| during pre-dosing and up-dosing periods | | | | |
| excluding V16 DBPCFCs | | | | |
| Maximum severity by Muraro Grading Scale | | | | |
| Mild | | | 0 | 3 (3.1%) |
| Moderate | | | 2 (4.0%) | 2 (2.0%) |
| Severe | | | 0 | 0 |
| Subjects experiencing an anaphylactic reaction | | | 0 | 3 (3.1%) |
| that required use of epinephrine | | | | |
| Unbalance PTs between two groups | | | | |
| Upper respiratory tract infection | 5 (10.0%) | 1 (1.0%) | PT Balanced | |
| Nasal congestion | PTs Balanced | | 0 | 4 (4.1%) |
| Asthma | | | 4 (8.0%) | 2 (2.0%) |
| Abdominal pain upper | | | 2 (4.0%) | 8 (8.2%) |
| Nausea | | | 6 (12.0%) | 8 (8.2%) |
| Diarrhea | | | 4 (8.0%) | 4 (4.1%) |
| Vomiting | | | 8 (16.0%) | 4 (4.1%) |
| Abdominal discomfort | | | 3 (6.0%) | 1 (1.0%) |
| Adverse drug reaction | | | 2 (4.0%) | 0 |
| Skin abrasion | | | 0 | 4 (4.1%) |
| Ear pain | | | 2 (4.0%) | 0 |

TABLE 12
Summary of Epinephrine Use for Treatment of Acute-Allergic Reactions

| | Placebo + AR101 | Dupilumab + AR101 | Total |
|---|---|---|---|
| Subjects with any epinephrine use | (N = 50) | (N = 98) | (N = 148) |
| Screening DBPCFCs (peanut challenge) | | | |
| Yes | 19 (38.0%) | 41 (41.8%) | 60 (40.5%) |
| No | 31 (62.0%) | 57 (58.2%) | 88 (59.5%) |
| Week 28 DBPCFCs (peanut challenge) | | | |
| Yes | 9 (18.0%) | 13 (13.3%) | 22 (14.9%) |
| No | 41 (82.0%) | 85 (86.7%) | 126 (85.1%) |
| Subjects with an episode requiring at least one | 0 | 0 | 0 |
| epinephrine use during pre-dosing period | | | |
| Subjects with an episode requiring at least one | 0 | 0 | 0 |
| epinephrine use during AR101 IDED | | | |
| Subjects with an episode requiring at least one | | | |
| epinephrine use during up-dosing period | | | |
| Subjects with 1 episode | 2 (4.0%) | 3 (3.1%) | 5 (3.4%) |
| Subjects with >1 episode | 0 | 0 | 0 |

Results from End-of Study Analysis

As described above, patients who achieved 300 mg/day AR101 for at least 2 weeks during the up-dosing period were eligible to enter a 24-week maintenance phase in which all subjects continued to receive AR101 300 mg/day. Subjects in the dupilumab treatment group were re-randomized to either receive dupilumab at the same dose as administered during the up-dosing period or to receive placebo. Subjects who received placebo during the up-dosing phase will continue to receive placebo in the maintenance phase. After 24 weeks of AR101 and either dupilumab or placebo, subjects underwent a post maintenance DBPCFC to assess the level of peanut sensitivity at the end of the maintenance period, followed by a 12-week safety follow-up period. See, FIG. 1.

For the mFAS-Maintenance group, there were a total of 104 patients (29 patients continuously on placebo+AR101; 36 patients continuously on dupilumab+AR101; and 39 patients on dupilumab+AR101 through the up-dosing phase and on placebo+AR101 for the maintenance phase). The demographic and baseline disease characteristics were generally balanced among the treatment groups, except the screening sIgE was higher for continuous placebo group (data not shown).

Continuing dupilumab treatment as an adjunct to AR101 for an additional 6 months numerically further increased the percentage of patients who achieved desensitization at week 52, as measured by passing a DBPCFC with 2044 mg (cumulative) peanut protein. For mFAS-Maintenance patients continuously on dupilumab+AR101, 63.9% (23/36) passed the DBPCFC at week 52; compare to 56.0% (47/84) of up-dosing mFAS patients as shown in Table 8. For patients who discontinued dupilumab after the up-dosing phase, there was a decrease in the percentage of subjects who passed the DBPCFC at week 52 (43.6% (17/39) of patients in the dupilumab+AR101/placebo+AR101 group). For the continuous placebo group, 48.2% (14/29) of patients passed the DBPCFC. The cumulative tolerated dose of peanut protein during the week 52 DBPCFC was not significantly different in the dupilumab+AR101 group as compared to AR101 alone.

In subjects with lower screening psIgE (<52.5 kU/L or <100 kU/L), a higher percentage of patients passed the 2044 mg cumulative tolerated dose of peanut protein at week 52 DBPCFC in the continuous dupilumab+AR101 group, as compared to the placebo+AR101 group (79% vs 30% for the <52.5 kU/L subgroup, and 76% vs. 36% for the <100 kU/L subgroup).

In the mFAS-Maintenance group, dupilumab was well tolerated with an acceptable safety profile during maintenance. TEAEs were similar between the placebo (42.5%) and continuous dupilumab (44.2%) groups, but was higher in the dupilumab+AR101/placebo+AR101 group (59.5%), including one SAE of appendicitis. Rescue medication use was comparable among the treatment groups during the maintenance period.

### CONCLUSION

In summary, dupilumab adjunct treatment showed clinically meaningful and statistically significant 20% improvement in the proportion of subjects who safely passed 2044 mg cumulative peanut protein, and a statistically significant increase in the cumulative tolerated dose (log transformed) of peanut protein during a post AR101 peanut OIT up-dosing DBPCFC in pediatric and adolescent subjects. Dupilumab as an adjunct to AR101 peanut OIT was generally safe and well tolerated in pediatric subjects with peanut allergy.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

TABLE 13
Informal Sequence Listing

| SEQ | | |
|---|---|---|
| ID NO | Sequence | Description |
| 1 | EVQLVESGGGLEQPGGSLRLSCAGSGFTFRDYAMTWVRQAPGKGLEWVSSISGSG | Dupilumab HCVR amino |
| | GNTYYADSVKGRFTISRDNSKNTLYLQMNSLRAEDTAVYYCAKDRLSITIRPRYYGLD | acid sequence |
| | VWGQGTTVTVS | |
| 2 | DIVMTQSPLSLPVTPGEPASISCRSSQSLLYSIGYNYLDWYLQKSGQSPQLLIYLGSNR | Dupilumab LCVR amino |
| | ASGVPDRFSGSGSGTDFTLKISRVEAEDVGFYYCMQALQTPYTFGQGTKLEIK | acid sequence |
| 3 | GFTFRDYA | Dupilumab HCDR1 |
| | | amino acid sequence |
| 4 | ISGSGGNT | Dupilumab HCDR2 |
| | | amino acid sequence |
| 5 | AKDRLSITIRPRYYGLDV | Dupilumab HCDR3 |
| | | amino acid sequence |
| 6 | QSLLYSIGYNY | Dupilumab LCDR1 amino |
| | | acid sequence |
| 7 | LGS | Dupilumab LCDR2 amino |
| | | acid sequence |
| 8 | MQALQTPYT | Dupilumab LCDR3 amino |
| | | acid sequence |
| 9 | EVQLVESGGGLEQPGGSLRLSCAGSGFTFRDYAMTWVRQAPGKGLEWVSSISGSG | Dupilumab heavy chain |
| | GNTYYADSVKGRFTISRDNSKNTLYLQMNSLRAEDTAVYYCAKDRLSITIRPRYYGLD | amino acid sequence |
| | VWGQGTTVTVSSASTKGPSVFPLAPCSRSTSESTAALGCLVKDYFPEPVTVSWNSGA | |
| | LTSGVHTFPAVLQSSGLYSLSSVVTVPSSSLGTKTYTCNVDHKPSNTKVDKRVESKYGP | |
| | PCPPCPAPEFLGGPSVFLFPPKPKDTLMISRTPEVTCVVVDVSQEDPEVQFNWYVDG | |
| | VEVHNAKTKPREEQFNSTYRVVSVLTVLHQDWLNGKEYKCKVSNKGLPSSIEKTISKA | |
| | KGQPREPQVYTLPPSQEEMTKNQVSLTCLVKGFYPSDIAVEWESNGQPENNYKTTP | |
| | PVLDSDGSFFLYSRLTVDKSRWQEGNVFSCSVMHEALHNHYTQKSLSLSLGK | |
| 10 | DIVMTQSPLSLPVTPGEPASISCRSSQSLLYSIGYNYLDWYLQKSGQSPQLLIYLGSNR | Dupilumab light chain |
| | ASGVPDRFSGSGSGTDFTLKISRVEAEDVGFYYCMQALQTPYTFGQGTKLEIKRTVAA | amino acid sequence |
| | PSVFIFPPSDEQLKSGTASVVCLLNNFYPREAKVQWKVDNALQSGNSQESVTEQDSK | |
| | DSTYSLSSTLTLSKADYEKHKVYACEVTHQGLSSPVTKSFNRGEC | |
| 11 | MKVLQEPTCVSDYMSISTCEWKMNGPTNCSTELRLLYQLVFLLSEAHTCIPENNGGA | Human IL-4Rα |
| | GCVCHLLMDDVVSADNYTLDLWAGQQLLWKGSFKPSEHVKPRAPGNLTVHTNVS | |
| | DTLLLTWSNPYPPDNYLYNHLTYAVNIWSENDPADFRIYNVTYLEPSLRIAASTLKSGI | |
| | SYRARVRAWAQCYNTTWSEWSPSTKWHNSYREPFEQH | |
| 12 | EIVLTQSPGTLSLSPGERATLSCRASQSVSNSYLAWYQQKPGQAPRLLIFGASSRATGI | SCB-VL-39 |
| | PDRFSGSGSGTDFTLTISRLEPEDFAVYYCQQYGSSPPWTFGQGTKVEIK | |
| 13 | EIVLTQSPGTLSLSPGERATLSCRASQSVSSSYLAWYQQKPGQAPRLLIYGASSRATGI | SCB-VL-40 |
| | PDRFSGSGSGTDFTLTISRLEPEDFAVYYCQQYGSSPPWTFGQGTKVEIK | |
| 14 | EIVLTQSPGTLSLSPGERATLSCRASQSVSSSYLAWYQQKPGQAPRLLIFGASSRAPGI | SCB-VL-41 |
| | PDRFSGSGSGTDFTLTISRLEPEDFAVYYCQQYGSSPPWTFGQGTKVEIK | |
| 15 | EIVLTQSPGTLSLSPGERATLSCRASQSVSNSYLAWYQQKPGQAPRLLIYGASSRATGI | SCB-VL-42 |
| | PDRFSGSGSGTDFTLTISRLEPEDFAVYYCQQYGSSPPWTFGQGTKVEIK | |
| 16 | EIVLTQSPGTLSLSPGERATLSCRASQSVSNSYLAWYQQKPGQAPRLLIFGASSRAPGI | SCB-VL-43 |
| | PDRFSGSGSGTDFTLTISRLEPEDFAVYYCQQYGSSPPWTFGQGTKVEIK | |
| 17 | EIVLTQSPGTLSLSPGERATLSCRASQSVSSSYLAWYQQKPGQAPRLLIYGASSRAPGI | SCB-VL-44 |
| | PDRFSGSGSGTDFTLTISRLEPEDFAVYYCQQYGSSPPWTFGQGTKVEIK | |
| 18 | EIVLTQSPGTLSLSPGERATLSCRASQSVSSSYLAWYQQKPGQAPRLLIFGASSRATGI | SCB-VL-45 |
| | PDRFSGSGSGTDFTLTISRLEPEDFAVYYCQQYDHSPPWTFGQGTKVEIK | |
| 19 | EIVLTQSPGTLSLSPGERATLSCRASQSVSSSYLAWYQQKPGQAPRLLIFGASSRATGI | SCB-VL-46 |
| | PDRFSGSGSGTDFTLTISRLEPEDFAVYYCQQYGSSAGWTFGQGTKVEIK | |
| 20 | EIVLTQSPGTLSLSPGERATLSCRASQSVSSSYLAWYQQKPGQAPRLLIFGASSRATGI | SCB-VL-47 |
| | PDRFSGSGSGTDFTLTISRLEPEDFAVYYCQQYDHSAGWTFGQGTKVEIK | |
| 21 | EIVLTQSPGTLSLSPGERATLSCRASQSVSNSYLAWYQQKPGQAPRLLIFGASSRATGI | SCB-VL-48 |
| | PDRFSGSGSGTDFTLTISRLEPEDFAVYYCQQYDHSPPWTFGQGTKVEIK | |
| 22 | EIVLTQSPGTLSLSPGERATLSCRASQSVSSSYLAWYQQKPGQAPRLLIYGASSRATGI | SCB-VL-49 |
| | PDRFSGSGSGTDFTLTISRLEPEDFAVYYCQQYDHSPPWTFGQGTKVEIK | |
| 23 | EIVLTQSPGTLSLSPGERATLSCRASQSVSSSYLAWYQQKPGQAPRLLIFGASSRAPGI | SCB-VL-50 |
| | PDRFSGSGSGTDFTLTISRLEPEDFAVYYCQQYDHSPPWTFGQGTKVEIK | |
| 24 | EIVLTQSPGTLSLSPGERATLSCRASQSVSSSYLAWYQQKPGQAPRLLIYGASSRAPGI | SCB-VL-51 |
| | PDRFSGSGSGTDFTLTISRLEPEDFAVYYCQQYDHSAGWTFGQGTKVEIK | |
| 25 | EIVLTQSPGTLSLSPGERATLSCRASQSVSNSYLAWYQQKPGQAPRLLIFGASSRAPGI | SCB-VL-52 |
| | PDRFSGSGSGTDFTLTISRLEPEDFAVYYCQQYDHSAGWTFGQGTKVEIK | |
| 26 | EIVLTQSPGTLSLSPGERATLSCRASQSVSNSYLAWYQQKPGQAPRLLIYGASSRATGI | SCB-VL-53 |
| | PDRFSGSGSGTDFTLTISRLEPEDFAVYYCQQYDHSAGWTFGQGTKVEIK | |
| 27 | EIVLTQSPGTLSLSPGERATLSCRASQSVSSSYLAWYQQKPGQAPRLLIFGASSRAPGI | SCB-VL-54 |
| | PDRFSGSGSGTDFTLTISRLEPEDFAVYYCQQYDHSAGWTFGQGTKVEIK | |
| 28 | EIVLTQSPGTLSLSPGERATLSCRASQSVSSSYLAWYQQKPGQAPRLLIYGASSRATGI | SCB-VL-55 |
| | PDRFSGSGSGTDFTLTISRLEPEDFAVYYCQQYDHSAGWTFGQGTKVEIK | |
| 29 | EIVLTQSPGTLSLSPGERATLSCRASQSVSNSYLAWYQQKPGQAPRLLIFGASSRATGI | SCB-VL-56 |
| | PDRFSGSGSGTDFTLTISRLEPEDFAVYYCQQYDHSAGWTFGQGTKVEIK | |
| 30 | EIVLTQSPGTLSLSPGERATLSCRASQSVSSSYLAWYQQKPGQAPRLLIFGASSRATGI | SCB-VL-57 |
| | PDRFSGSGSGTDFTLTISRLEPEDFAVYYCQQYGSSPPWTFGQGTKVEIK | |
| 31 | EIVLIQSPGILSLSPGERATLSCRASQSVSNSYLAWYQQKPGQAPRLLIYGASSRAPGI | SCB-VL-58 |
| | PDRFSGSGSGTDFTLTISRLEPEDFAVYYCQQYDHSAGWTFGQGTKVEIK | |
| 32 | EVQLVESGGGLVHPGGSLRLSCAGSGFTFSRNAMFWVRQAPGKGLEWVSGIGTGG | SCB-VH-59 |
| | ATNYADSVKGRFTISRDNAKNSLYLQMNSLRAEDMAVYYCARGRYYFDYWGQGTL | |
| | VTVSS | |
| 33 | EVQLVQSGGGLVQPGGSLRLSCAGSGFTFSRNAMFWVRQAPGKGLEWVSGIGTG | SCB-VH-60 |
| | GATNYADSVKGRFTISRDNAKNSLYLQMNSLRAEDMAVYYCARGRYYFDYWGQGT | |
| | LVTVSS | |
| 34 | EVQLVQSGGGLVHPGGSLRLSCAASGFTFSRNAMFWVRQAPGKGLEWVSGIGTGG | SCB-VH-61 |
| | ATNYADSVKGRFTISRDNAKNSLYLQMNSLRAEDMAVYYCARGRYYFDYWGQGTL | |
| | VTVSS | |
| 35 | EVQLVQSGGGLVHPGGSLRLSCAGSGFTFSRNAMFWVRQAPGKGLEWVSGIGTGG | SCB-VH-62 |
| | ATSYADSVKGRFTISRDNAKNSLYLQMNSLRAEDMAVYYCARGRYYFDYWGQGTLV | |
| | TVSS | |
| 36 | EVQLVQSGGGLVHPGGSLRLSCAGSGFTFSRNAMFWVRQAPGKGLEWVSGIGTGG | SCB-VH-63 |
| | ATNYADSVKGRFTISRDNAKNSLYLQMNSLRAEDTAVYYCARGRYYFDYWGQGTLV | |
| | TVSS | |
| 37 | EVQLVESGGGLVQPGGSLRLSCAGSGFTFSRNAMFWVRQAPGKGLEWVSGIGTGG | SCB-VH-64 |
| | ATNYADSVKGRFTISRDNAKNSLYLQMNSLRAEDMAVYYCARGRYYFDYWGQGTL | |
| | VTVSS | |
| 38 | EVQLVESGGGLVHPGGSLRLSCAASGFTFSRNAMFWVRQAPGKGLEWVSGIGTGG | SCB-VH-65 |
| | ATNYADSVKGRFTISRDNAKNSLYLQMNSLRAEDMAVYYCARGRYYFDYWGQGTL | |
| | VTVSS | |
| 39 | EVQLVQSGGGLVQPGGSLRLSCAASGFTFSRNAMFWVRQAPGKGLEWVSGIGTGG | SCB-VH-66 |
| | ATNYADSVKGRFTISRDNAKNSLYLQMNSLRAEDMAVYYCARGRYYFDYWGQGTL | |
| | VTVSS | |
| 40 | EVQLVQSGGGLVHPGGSLRLSCAGSGFTFSRNAMFWVRQAPGKGLEWVSGIGTGG | SCB-VH-67 |
| | ATSYADSVKGRFTISRDNAKNSLYLQMNSLRAEDTAVYYCARGRYYFDYWGQGTLV | |
| | TVSS | |
| 41 | EVQLVQSGGGLVHPGGSLRLSCAGSGFTFSRNAMFWVRQAPGKGLEWVSGIGTGG | SCB-VH-68 |
| | ATNYADSVKGRFTISRDNAKNSLYLQMNSLRAEDMAVYYCARGRYYFPWWGQGTL | |
| | VTVSS | |
| 42 | EVQLVESGGGLVHPGGSLRLSCAGSGFTFSRNAMFWVRQAPGKGLEWVSGIGTGG | SCB-VH-69 |
| | ATNYADSVKGRFTISRDNAKNSLYLQMNSLRAEDMAVYYCARGRYYFPWWGQGTL | |
| | VTVSS | |
| 43 | EVQLVQSGGGLVQPGGSLRLSCAGSGFTFSRNAMFWVRQAPGKGLEWVSGIGTG | SCB-VH-70 |
| | GATNYADSVKGRFTISRDNAKNSLYLQMNSLRAEDMAVYYCARGRYYFPWWGQG | |
| | TLVTVSS | |
| 44 | EVQLVQSGGGLVHPGGSLRLSCAASGFTFSRNAMFWVRQAPGKGLEWVSGIGTGG | SCB-VH-71 |
| | ATNYADSVKGRFTISRDNAKNSLYLQMNSLRAEDMAVYYCARGRYYFPWWGQGTL | |
| | VTVSS | |
| 45 | EVQLVQSGGGLVHPGGSLRLSCAGSGFTFSRNAMFWVRQAPGKGLEWVSGIGTGG | SCB-VH-72 |
| | ATSYADSVKGRFTISRDNAKNSLYLQMNSLRAEDMAVYYCARGRYYFPWWGQGTL | |
| | VTVSS | |
| 46 | EVQLVQSGGGLVHPGGSLRLSCAGSGFTFSRNAMFWVRQAPGKGLEWVSGIGTGG | SCB-VH-73 |
| | ATNYADSVKGRFTISRDNAKNSLYLQMNSLRAEDTAVYYCARGRYYFPWWGQGTL | |
| | VTVSS | |
| 47 | EVQLVQSGGGLVHPGRSLRLSCAGSGFTFSRNAMFWVRQAPGKGLEWVSGIGTGG | SCB-VH-74 |
| | ATNYADSVKGRFTISRDNAKNSLYLQMNSLRAEDMAVYYCARGRYYFDYWGQGTL | |
| | VTVSS | |
| 48 | EVQLVQSGGGLVHPGGSLRLTCAGSGFTFSRNAMFWVRQAPGKGLEWVSGIGTG | SCB-VH-75 |
| | GATNYADSVKGRFTISRDNAKNSLYLQMNSLRAEDMAVYYCARGRYYFDYWGQGT | |
| | LVTVSS | |
| 49 | EVQLVQSGGGLVHPGGSLRLSCAGSGFTFSRNAMHWVRQAPGKGLEWVSGIGTG | SCB-VH-76 |
| | GATNYADSVKGRFTISRDNAKNSLYLQMNSLRAEDMAVYYCARGRYYFDYWGQGT | |
| | LVTVSS | |
| 50 | EVQLVQSGGGLVHPGGSLRLSCAGSGFTFSRNAMFWVRQAPGEGLEWVSGIGTGG | SCB-VH-77 |
| | ATNYADSVKGRFTISRDNAKNSLYLQMNSLRAEDMAVYYCARGRYYFDYWGQGTL | |
| | VTVSS | |
| 51 | EVQLVQSGGGLVHPGGSLRLSCAGSGFTFSRNAMFWVRQAPGKGLEWVSGIGTGG | SCB-VH-78 |
| | ATNYADSVKGRFTISRDEAKNSLYLQMNSLRAEDMAVYYCARGRYYFDYWGQGTLV | |
| | TVSS | |
| 52 | EVQLVQSGGGLVHPGGSLRLSCAGSGFTFSRNAMFWVRQAPGKGLEWVSGIGTGG | SCB-VH-79 |
| | ATNYADSVKGRFTISRDNAKNSLYLQMNSLRAGDMAVYYCARGRYYFDYWGQGTL | |
| | VTVSS | |
| 53 | EVQLVQSGGGLVHPGGSLRLSCAGSGFTFDDYAMFWVRQAPGKGLEWVSGIGTG | SCB-VH-80 |
| | GATNYADSVKGRFTISRDNAKNSLYLQMNSLRAEDMAVYYCARGRYYFDYWGQGT | |
| | LVTVSS | |
| 54 | EVQLVQSGGGLVQPGGSLRLSCAASGFTFSRNAMFWVRQAPGKGLEWVSGIGTGG | SCB-VH-81 |
| | ATSYADSVKGRFTISRDNAKNSLYLQMNSLRAEDTAVYYCARGRYYFPWWGQGTLV | |
| | TVSS | |
| 55 | EVQLVESGGGLVHPGGSLRLSCAASGFTFSRNAMFWVRQAPGKGLEWVSGIGTGG | SCB-VH-82 |
| | ATSYADSVKGRFTISRDNAKNSLYLQMNSLRAEDTAVYYCARGRYYFPWWGQGTLV | |
| | TVSS | |
| 56 | EVQLVESGGGLVQPGGSLRLSCAGSGFTFSRNAMFWVRQAPGKGLEWVSGIGTGG | SCB-VH-83 |
| | ATSYADSVKGRFTISRDNAKNSLYLQMNSLRAEDTAVYYCARGRYYFPWWGQGTLV | |
| | TVSS | |
| 57 | EVQLVESGGGLVQPGGSLRLSCAASGFTFSRNAMFWVRQAPGKGLEWVSGIGTGG | SCB-VH-84 |
| | ATNYADSVKGRFTISRDNAKNSLYLQMNSLRAEDTAVYYCARGRYYFPWWGQGTL | |
| | VTVSS | |
| 58 | EVQLVESGGGLVQPGGSLRLSCAASGFTFSRNAMFWVRQAPGKGLEWVSGIGTGG | SCB-VH-85 |
| | ATSYADSVKGRFTISRDNAKNSLYLQMNSLRAEDMAVYYCARGRYYFPWWGQGTL | |
| | VTVSS | |
| 59 | EVQLVQSGGGLVHPGGSLRLSCAASGFTFSRNAMFWVRQAPGKGLEWVSGIGTGG | SCB-VH-86 |
| | ATSYADSVKGRFTISRDNAKNSLYLQMNSLRAEDTAVYYCARGRYYFPWWGQGTLV | |
| | TVSS | |
| 60 | EVQLVQSGGGLVQPGGSLRLSCAGSGFTFSRNAMFWVRQAPGKGLEWVSGIGTG | SCB-VH-87 |
| | GATSYADSVKGRFTISRDNAKNSLYLQMNSLRAEDTAVYYCARGRYYFPWWGQGTL | |
| | VTVSS | |
| 61 | EVQLVESGGGLVHPGGSLRLSCAGSGFTFSRNAMFWVRQAPGKGLEWVSGIGTGG | SCB-VH-88 |
| | ATSYADSVKGRFTISRDNAKNSLYLQMNSLRAEDTAVYYCARGRYYFPWWGQGTLV | |
| | TVSS | |
| 62 | EVQLVQSGGGLVHPGGSLRLSCAGSGFTFSRNAMFWVRQAPGKGLEWVSGIGTGG | SCB-VH-89 |
| | ATSYADSVKGRFTISRDNAKNSLYLQMNSLRAEDTAVYYCARGRYYFPWWGQGTLV | |
| | TVSS | |
| 63 | EVQLVESGGGLVQPGGSLRLSCAASGFTFSRNAMFWVRQAPGKGLEWVSGIGTGG | SCB-VH-90 |
| | ATNYADSVKGRFTISRDNAKNSLYLQMNSLRAEDMAVYYCARGRYYFPWWGQGTL | |
| | VTVSS | |
| 64 | EVQLVESGGGLVQPGGSLRLSCAASGFTFSRNAMFWVRQAPGKGLEWVSGIGTGG | SCB-VH-91 |
| | ATSYADSVKGRFTISRDNAKNSLYLQMNSLRAEDTAVYYCARGRYYFDYWGQGTLV | |
| | TVSS | |
| 65 | EVQLVQSGGGLVHPGGSLRLSCAGSGFTFSRNAMFWVRQAPGKGLEWVSGIGTGG | SCB-VH-92 |
| | ATNYADSVKGRFTISRDNAKNSLYLQMNSLRAEDMAVYYCARGRYYFDYWGQGTL | |
| | VTVSS | |
| 66 | EVQLVESGGGLVQPGGSLRLSCAASGFTFSRNAMFWVRQAPGKGLEWVSGIGTGG | SCB-VH-93 |
| | ATSYADSVKGRFTISRDNAKNSLYLQMNSLRAEDTAVYYCARGRYYFPWWGQGTLV | |
| | TVSS | |
| 67 | QVQLVQSGAEVKKPGASVKVSCKASGYAFTSYYMHWARQAPGQGLEWMGIINPS | MEDI-1-VH |
| | GGSTSYAQKFQGRVTMTRDTSTSTVYMELSSLRSEDTAVYYCARGKWWLDYWGKG | |
| | TLVTVSS | |
| 68 | QSVLTQPPSVSAAPGQKVTISCSGGSSNIGNSYVSWYQQLPGTAPKLLIYDNNKRPS | MEDI-1-VL |
| | GIPDRFSGSKSGTSATLAITGLQTGDEADYYCGTWDTSLSANYVEGIGTKLTVL | |
| 69 | QVQLVQSGAEVKKPGASVKVSCKASGYAFTSYYMHWARQAPGQGLEWMGIINPS | MEDI-2-VH |
| | GGSTSYAQKFQGRVTMTRDTSTSTVYMELSSLRSEDTAVYYCARGKWWLYNWGKG | |
| | TLVTVSS | |
| 70 | QSVLTQPPSVSAAPGQKVTISCSGGSSNIGNSYVSWYQQLPGTAPKLLIYDNNKRPS | MEDI-2-VL |
| | GIPDRFSGSKSGTSATLAITGLQTGDEADYYCGTWDTSQPPNPLFGTGTKLTVL | |
| 71 | QVQLVQSGAEVKKPGASVKVSCKASGYAFTSYYMHWARQAPGQGLEWMGIINPS | MEDI-3-VH |
| | GGSTSYAQKFQGRVTMTRDTSTSTVYMELSSLRSEDTAVYYCARGKLLKNPWGKGT | |
| | LVTVSS | |
| 72 | QSVLTQPPSVSAAPGQKVTISCSGGSSNIGNSYVSWYQQLPGTAPKLLIYDNNKRPS | MEDI-3-VL |
| | GIPDRFSGSKSGTSATLAITGLQTGDEADYYCGTWFGTPASNYVFGTGTKLTVL | |
| 73 | QVQLVQSGAEVKKPGASVKVSCKASGYAFTSYYMHWARQAPGQGLEWMGIINPS | MEDI-4-VH |
| | GGSTSYAQKFQGRVTMTRDTSTSTVYMELSSLRSEDTAVYYCARGKWWLYNWGKG | |
| | TLVTVSS | |
| 74 | QSVLTQPPSVSAAPGQKVTISCSGGSSNIGNSYVSWYQQLPGTAPKLLIYDNNKRPS | MEDI-4-VL |
| | GIPDRFSGSKSGTSATLAITGLQTGDEADYYCGTWDTSSPPQPIFGTGTKLTVL | |
| 75 | QVQLVQSGAEVKKPGASVKVSCKASGYAFTSYYMHWARQAPGQGLEWMGIINPS | MEDI-5-VH |
| | GGSTSYAQKFQGRVTMTRDTSTSTVYMELSSLRSEDTAVYYCARGKWWLYDWGKG | |
| | TLVTVSS | |
| 76 | QSVLTQPPSVSAAPGQKVTISCSGGSSNIGNSYVSWYQQLPGTAPKLLIYDNNKRPS | MEDI-5-VL |
| | GIPDRFSGSKSGTSATLAITGLQTGDEADYYCGTWDTSSPPQPIFGTGTKLTVL | |
| 77 | QVQLVQSGAEVKKPGASVKVSCKASGYAFTSYYMHWARQAPGQGLEWMGIINPS | MEDI-6-VH |
| | GGSTSYAQKFQGRVTMTRDTSTSTVYMELSSLRSEDTAVYYCARGKYWMYDWGKG | |
| | TLVTVSS | |
| 78 | QSVLTQPPSVSAAPGQKVTISCSGGSSNIGNSYVSWYQQLPGTAPKLLIYDNNKRPS | MEDI-6-VL |
| | GIPDRFSGSKSGTSATLAITGLQTGDEADYYCGTWDTSTTYHPIFGTGTKLTVL | |
| 79 | QVQLVQSGAEVKKPGASVKVSCKASGYAFTSYYMHWARQAPGQGLEWMGIINPS | MEDI-7-VH |
| | GGSTSYAQKFQGRVTMTRDTSTSTVYMELSSLRSEDTAVYYCARGKWWWQYWGK | |
| | GTLVTVSS | |
| 80 | QSVLTQPPSVSAAPGQKVTISCSGGSSNIGNSYVSWYQQLPGTAPKLLIYDNNKRPS | MEDI-7-VL |
| | GIPDRFSGSKSGTSATLAITGLQTGDEADYYCGTWDTSSPPQPIFGTGTKLTVL | |
| 81 | QVQLVQSGAEVKKPGASVKVSCKASGYAFTSYYMHWARQAPGQGLEWMGIINPS | MEDI-8-VH |
| | GGSTSYAQKFQGRVTMTRDTSTSTVYMELSSLRSEDTAVYYCARGKWWWQYWGK | |
| | GTLVTVSS | |
| 82 | QSVLTQPPSVSAAPGQKVTISCSGGSSNIGNSYVSWYQQLPGTAPKLLIYDNNKRPS | MEDI-8-VL |
| | GIPDRFSGSKSGTSATLAITGLQTGDEADYYCGTWDTSTTYHPIFGTGTKLTVL | |
| 83 | QVQLVQSGAEVKKPGASVKVSCKASGYAFTSYYMHWARQAPGQGLEWMGIINPS | MEDI-9-VH |
| | GGSTSYAQKFQGRVTMTRDTSTSTVYMELSSLRSEDTAVYYCARGKWWLYNWGKG | |
| | TLVTVSS | |
| 84 | QSVLTQPPSVSAAPGQKVTISCSGGSSNIGNSYVSWYQQLPGTAPKLLIYDNNKRPS | MEDI-9-VL |
| | GIPDRFSGSKSGTSATLAITGLQTGDEADYYCGTWDTSTTMYPLFGTGTKLTVL | |
| 85 | QVQLVQSGAEVKKPGASVKVSCKASGYAFTSYYMHWARQAPGQGLEWMGIINPS | MEDI-10-VH |
| | GGSTSYAQKFQGRVTMTRDTSTSTVYMELSSLRSEDTAVYYCARGKWWLYDWGKG | |
| | TLVTVSS | |
| 86 | QSVLTQPPSVSAAPGQKVTISCSGGSSNIGNSYVSWYQQLPGTAPKLLIYDNNKRPS | MEDI-10-VL |
| | GIPDRFSGSKSGTSATLAITGLQTGDEADYYCGTWDTSTVLTPIFGTGTKLTVL | |
| 87 | QVQLVQSGAEVKKPGASVKVSCKASGYAFTSYYMHWARQAPGQGLEWMGIINPS | MEDI-11-VH |
| | GGSTSYAQKFQGRVTMTRDTSTSTVYMELSSLRSEDTAVYYCARGKWWFYDWGKG | |
| | TLVTVSS | |
| 88 | QSVLTQPPSVSAAPGQKVTISCSGGSSNIGNSYVSWYQQLPGTAPKLLIYDNNKRPS | MEDI-11-VL |
| | GIPDRFSGSKSGTSATLAITGLQTGDEADYYCGTWDTSPSMIPLFGTGTKLTVL | |
| 89 | QVQLVQSGAEVKKPGASVKVSCKASGYAFTSYYMHWARQAPGQGLEWMGIINPS | MEDI-12-VH |
| | GGSTSYAQKFQGRVTMTRDTSTSTVYMELSSLRSEDTAVYYCARGKWWFYDWGKG | |
| | TLVTVSS | |
| 90 | QSVLTQPPSVSAAPGQKVTISCSGGSSNIGNSYVSWYQQLPGTAPKLLIYDNNKRPS | MEDI-12-VL |
| | GIPDRFSGSKSGTSATLAITGLQTGDEADYYCGTWDTSTTMYPLFGTGTKLTVL | |
| 91 | QVQLVQSGAEVKKPGASVKVSCKASGYAFTSYYMHWARQAPGQGLEWMGIINPS | MEDI-13-VH |
| | GGSTSYAQKFQGRVTMTRDTSTSTVYMELSSLRSEDTAVYYCARGKWWLYDWGKG | |
| | TLVTVSS | |
| 92 | QSVLTQPPSVSAAPGQKVTISCSGGSSNIGNSYVSWYQQLPGTAPKLLIYDNNKRPS | MEDI-13-VL |
| | GIPDRFSGSKSGTSATLAITGLQTGDEADYYCGTWDTSTTLQPLFGTGTKLTVL | |
| 93 | QVQLVQSGAEVKKPGASVKVSCKASGYAFTSYYMHWARQAPGQGLEWMGIINPS | MEDI-14-VH |
| | GGSTSYAQKFQGRVTMTRDTSTSTVYMELSSLRSEDTAVYYCARGKWWLYNWGKG | |
| | TLVTVSS | |
| 94 | QSVLTQPPSVSAAPGQKVTISCSGGSSNIGNSYVSWYQQLPGTAPKLLIYDNNKRPS | MEDI-14-VL |
| | GIPDRFSGSKSGTSATLAITGLQTGDEADYYCGTWDTSPPTKPLFGTGTKLTVL | |
| 95 | QVQLVQSGAEVKKPGASVKVSCKASGYAFTSYYMHWARQAPGQGLEWMGIINPS | MEDI-15-VH |
| | GGSTSYAQKFQGRVTMTRDTSTSTVYMELSSLRSEDTAVYYCARGKWWLYNWGKG | |
| | TLVTVSS | |
| 96 | QSVLTQPPSVSAAPGQKVTISCSGGSSNIGNSYVSWYQQLPGTAPKLLIYDNNKRPS | MEDI-15-VL |
| | GIPDRFSGSKSGTSATLAITGLQTGDEADYYCGTWDTSTHRHPLFGTGTKLTVL | |
| 97 | QVQLVQSGAEVKKPGASVKVSCKASGYAFTSYYMHWARQAPGQGLEWMGIINPS | MEDI-16-VH |
| | GGSTSYAQKFQGRVTMTRDTSTSTVYMELSSLRSEDTAVYYCARGKWWLYNWGKG | |
| | TLVTVSS | |
| 98 | QSVLTQPPSVSAAPGQKVTISCSGGSSNIGNSYVSWYQQLPGTAPKLLIYDNNKRPS | MEDI-16-VL |
| | GIPDRFSGSKSGTSATLAITGLQTGDEADYYCGTWDTSTTYHPIFGTGTKLTVL | |
| 99 | QVQLVQSGAEVKKPGASVKVSCKASGYAFTSYYMHWARQAPGQGLEWMGIINPS | MEDI-17-VH |
| | GGSTSYAQKFQGRVTMTRDTSTSTVYMELSSLRSEDTAVYYCARGKWWWQHWGK | |
| | GTLVTVSS | |
| 100 | QSVLTQPPSVSAAPGQKVTISCSGGSSNIGNSYVSWYQQLPGTAPKLLIYDNNKRPS | MEDI-17-VL |
| | GIPDRFSGSKSGTSATLAITGLQTGDEADYYCGTWDTSPVDRP1 FGTGTKLTVL | |
| 101 | QVQLVQSGAEVKKPGASVKVSCKASGYAFTSYYMHWARQAPGQGLEWMGIINPS | MEDI-18-VH |
| | GGSTSYAQKFQGRVTMTRDTSTSTVYMELSSLRSEDTAVYYCARGKWWWQHWGK | |
| | GTLVTVSS | |
| 102 | QSVLTQPPSVSAAPGQKVTISCSGGSSNIGNSYVSWYQQLPGTAPKLLIYDNNKRPS | MEDI-18-VL |
| | GIPDRFSGSKSGTSATLAITGLQTGDEADYYCGTWDTSTTPMPVEGIGTKLTVL | |
| 103 | QVQLVQSGAEVKKPGASVKVSCKASGYAFTSYYMHWARQAPGQGLEWMGIINPS | MEDI-19-VH |
| | GGSTSYAQKFQGRVTMTRDTSTSTVYMELSSLRSEDTAVYYCARGKWWWQHWGK | |
| | GTLVTVSS | |
| 104 | QSVLTQPPSVSAAPGQKVTISCSGGSSNIGNSYVSWYQQLPGTAPKLLIYDNNKRPS | MEDI-19-VL |
| | GIPDRFSGSKSGTSATLAITGLQTGDEADYYCGTWDTSTTYHPIFGTGTKLTVL | |
| 105 | QVQLVQSGAEVKKPGASVKVSCKASGYAFTSYYMHWARQAPGQGLEWMGIINPS | MEDI-20-VH |
| | GGSTSYAQKFQGRVTMTRDTSTSTVYMELSSLRSEDTAVYYCARGKYWMYDWGKG | |
| | TLVTVSS | |
| 106 | QSVLTQPPSVSAAPGQKVTISCSGGSSNIGNSYVSWYQQLPGTAPKLLIYDNNKRPS | MEDI-20-VL |
| | GIPDRFSGSKSGTSATLAITGLQTGDEADYYCGTWDTSTVWEWPFGTGTKLTVL | |
| 107 | QVQLVQSGAEVKKPGASVKVSCKASGYAFTSYYMHWARQAPGQGLEWMGIINPS | MEDI-21-VH |
| | GGSASYAQKFQGRVTMTRDTSTSTVYMELSSLRSEDTAVYYCARGKYWMYDWGK | |
| | GTLVTVSS | |
| 108 | QSVLTQPPSVSAAPGQKVTISCSGGSSNIGNSYVSWYQQLPGTAPKLLIYDNNKRPS | MEDI-21-VL |
| | GIPDRFSGSKSGTSATLAITGLQTGDEAVYFCGTWDTSTVWEWPFGTGTKLTVL | |
| 109 | QVQLVQSGAEVKKPGASVKVSCKASGYAFTSYYMHWARQAPGQGLEWMGIINPS | MEDI-22-VH |
| | GGSTSYAQKFQGRVTMTRDTSTSTVYMELSSLRSEDTAVYYCARGKYWMYDWGKG | |
| | TLVTVSS | |
| 110 | QPVLTQPPSVSAAPGQKVTISCSGGSSNIGNSYVSWYQQLPGTAPKLLIYDNNKRPS | MEDI-22-VL |
| | GIPDRFSGSKSGTSATLAITGLQTGDEADYFCGTWDTSTVWEWPFGTGTKLTVL | |
| 111 | QVQLVQSGAEVRKPGASVKVSCKASGYAFTSYYMHWARQAPGQGLEWMGIINPS | MEDI-23-VH |
| | GGSTSYAQKFQGRVTMTRDTSTSTVYMELSSLRSEDTAVYYCARGKYWMYDWGKG | |
| | TLVTVSS | |
| 112 | QSVLTQPPSVSAAPGQKVTISCSGGSSNIGNNYVSWYQQLPGTAPKLLIYDNNKRPP | MEDI-23-VL |
| | GIPDRFSGSKSGTSATLAITGLQTGDEADYYCGTWDTSTVWEWPFGTGTKLTVL | |
| 113 | QVQLVQSGAEVKKPGASVKVSCKASGYAFTSYYMHWARQAPGQGLEWMGIINPR | MEDI-24-VH |
| | GGSTSYAQKFQGRVTMTRDTSTSTVYMELSSLRSEDTAVYYCARGKYWMYDWGKG | |
| | TLVTVSS | |
| 114 | QSVLTQPPSVSAAPGQKVTISCSGGSSNIGNSYVSWYQQLPGTAPKLLIYDNNKRPS | MEDI-24-VL |
| | GIPDRFSGSKSGTSATLAITGLQTGDEADYFCGTWDTSTVWEWPFGTGTKLTVL | |
| 115 | QVQLVQSGAEVKKPGASVKVSCKASGYAFTSYYMHWARQAPGQGLEWMGIINPR | MEDI-25-VH |
| | GGSASYAQKFQGRVSMTRDTSTSTVYMELSSLRSEDTAVYYCARGKYWMYDWGK | |
| | GTLVTVSS | |
| 116 | QSVLTQPPSVSAAPGQKVTISCSGGSSNIGNSYVSWYQQLPGTAPKLLIYDNNKRPS | MEDI-25-VL |
| | GIPDRFSGSKSGTTATLAITGLQTGDEADYYCGTWVTSTVWEWPFGTGTKLTVL | |
| 117 | QVQLVQSGAEVKKPGASVKVSCKASGYAFTSYYMHWARQAPGQGLEWMGIINPS | MEDI-26-VH |
| | GGSTSYAQKFQGRVTMTRDTSTSTVYMELSSLRSEDTAVYYCARGKYWMYDWGKG | |
| | TLVTVSS | |
| 118 | QSVLTQPPSVSAAPGQKVTISCSGGSSNIGNSYVSWYQQLPGTAPKLLIYDNNKRPS | MEDI-26-VL |
| | GIPDRFSGSKSGTSATLAITGLQTGDEADYFCGTWDTSTVWEWPFGTGTKLTVL | |
| 119 | QVQLVQSGAEVRKPGASVKVSCKASGYAFTSYYMHWARQAPGQGLEWMGIINPS | MEDI-27-VH |
| | GGSTSYAQKFQGRVTMTRDTSTSTVYMELSSLRPEDTAVYYCARGKYWMYDWGK | |
| | GTQVTVSS | |
| 120 | QSVLTQPPLVSAAPGQKVTISCSGGSSNIGNSYVSWYQRLPGTAPKLLIYDNNKRPSG | MEDI-27-VL |
| | IPDRFSGSKSGTSATLAITGLQTGDEADYYCGTWDTSTVWEWPFGTGTKLTVL | |
| 121 | QVQLVQSGAEVKKPGASVKVSCKASGYAFTSYYMHWARQAPGQGLEWMGIINPS | MEDI-28-VH |
| | GGSTSYAQKFQGRVTMTRDTSTSTVYMELSSLRSEDTAVYYCARGKYWMYDWGN | |
| | GTLVTVSS | |
| 122 | LPVLTQPPSVSAAPGQKVTISCSGGSSSIGNSYVSWYQQLPGAAPKLLIYDNNKRPSG | MEDI-28-VL |
| | IPDRFSGFRSGTSATLAITGLQTGDEADYYCGTWDTSPVWEWPFGTGTKLTVL | |
| 123 | QVQLVQSGAEVKKPGASVKVSCKASGYAFTSYYMHWARQAPGQGLEWMGIINPS | MEDI-29-VH |
| | GGSTSYAQKFQGRVTMTRDTSTSTVYMELSSLRSEDTAVYYCARGKYWMYDWGKG | |
| | TRVTVSS | |
| 124 | QSVLTQPPSVSAAPGQKVTISCSGGSSNIGNSYVSWYQQLPGTAPKLLIYDNNKRPS | MEDI-29-VL |
| | GIPDRFSGSKSGTSATLAITGLQTGDEADYYCGTWDTSPVWEWPFGTGTKLTVL | |
| 125 | QVQLVQSGAEVKKPGASVKVSCKASGYAFTSYYMHWARQAPGQGLEWMGIINPS | MEDI-30-VH |
| | GGSTSYAQKFQGRVTMTRDTSTSTVYMELSSLRSEDTAVYYCARGKYWMYDWGKG | |
| | TLVTVSS | |
| 126 | QSVLTQPPSVSAAPGQKVTISCSGGSSNIGNSYVSWYQRLPGAAPKLLIYDNNKRPS | MEDI-30-VL |
| | GIPDRFSGSKSGTSATLAITGLQTGDEADYYCGTWDTSTVWEWPFGTGTKLTVL | |
| 127 | QVQLVQSGAEVKKPGASVKVSCKASGYAFTSYYMHWARQAPGQGLEWMGIINPS | MEDI-31-VH |
| | GGSTSYAQKFQGRVTMTRDTSTSTVYMELSSLRSEDTAVYYCARGKYWMYDWGKG | |
| | TLVTVSS | |
| 128 | QSVLTQPPSVSAAPGQKVTISCSGGSSSIGNSYVSWYQQLPGTAPKLLIYDNNKRPSG | MEDI-31-VL |
| | IPDRFSGSKSGTSATLAITGLQTGDEADYYCGTWATSPVWEWPFGTGTKLTVL | |
| 129 | QVQLVQSGAEVKKPGASVKVSCKASGYAFTSYYMHWARQAPGQGLEWMGIINPS | MEDI-32-VH |
| | GGSTSYAQKFQGRVTMTRDTSTSTVYMELSSLRSEDTAVYYCARGKYWMYDWGKG | |
| | TLVTVSS | |
| 130 | QSVLTQPPSVSAAPGQKVTISCSGGSSNIGNSYVSWYQQLPGTAPKLLIYDNNKRPS | MEDI-32-VL |
| | GIPDRFSGSKSGTSATLAITGLQTGDEADYFCGTWDTSTAWEWPFGTGTKLTVL | |
| 131 | QVQLVQSGAEEKKPGASVKVSCKASGYAFTSYYMHWARQAPGQGLEWMGIINPS | MEDI-33-VH |
| | GGSTSYAQKFQGRVTMTRDTSTSTVYMELSSLRSEDTAVYYCARGKYWMYDWGKG | |
| | TLVTVSS | |
| 132 | QSALTQPPSVSAAPGQKVTISCSGGSSNIGNSYVSWYQQLPGTAPKLLIYDNNKRPS | MEDI-33-VL |
| | GIPDRFSGSKSGTSATLAITGLQTGDEADYFCGTWDTSTVWEWPFGTGTKLTVL | |
| 133 | QVQLVQSGAEVKKPGASVKVSCKASGYAFTSYYMHWARQAPGQGLEWMGIINPS | MEDI-34-VH |
| | GGSTSYAQKFQGRVSMTRDTSTSTVYMELSSLRSEDTAVYYCARGKYWMYDWGKG | |
| | TLVTVSS | |
| 134 | QSVLTQPPSVSAAPGQKVTISCSGGSSNIGNSYVSWYQQLPGTAPKLLIYDNNKRPS | MEDI-34-VL |
| | GIPDRFSGSKSGTSATLAITGLQTGDEADYFCGTWDTSTVWEWPFGTGTKLTVL | |
| 135 | QVQLVQSGAEVKKPGASVKVSCKASGYAFTSYYMHWARQAPGQGLEWMGIINPS | MEDI-35-VH |
| | GGSTSYAQKFQGRVTMTRDTSTSTVYMELSSLRSEDTAVYYCARGKYWMYDWGKG | |
| | TLVTVSS | |
| 136 | QSVLTQPPSVSAAPGQKVTISCSGGSSNIGNSYVSWYQQLPGTAPKLLIYDNNKRPS | MEDI-35-VL |
| | GIPDRFSGSKSGTSATLAITGLQTGDEADYYCGTWDTSPVWEWPFGTGTKLTVL | |
| 137 | QVQLVQSGAEVKKPGASVKVSCKASGYAFTSYYMHWARQAPGQGLEWMGIINPS | MEDI-36-VH |
| | GGSASYAQKFQGRVTMTRDTSTSTVYMELSSLRSEDTAVYYCARGKYWMYDWGK | |
| | GTLVTVSS | |
| 138 | QSVLTQPPSVSAAPGQKVTISCSGGSSNIGNSYVSWYQQLPGTAPKLLIYDNNKRPS | MEDI-36-VL |
| | GIPDRFSGSKSGTSATLAITGLQTGDEADYYCGTWDSSTVWEWPFGTGTKLTVL | |
| 139 | QVQLVQSGAEVKKPGASVKVSCKASGYAFTSYYMHWARQAPGQGLEWMGIINPR | MEDI-37-VH |
| | GGSTSYAQKFQGRVAMTRDTSTSTVYMELSSLRPEDTAVYYCARGKYWMYDWGK | |
| | GTLVTVSS | |
| 140 | QSVLTQPPSVSAAPGQKVTISCSGGGSSIGNSYVSWYQQLPGTAPKLLIYDNNKRPS | MEDI-37-VL |
| | GVPDRFSGSKSGTSATLAITGLQTGDEADYYCGTWDTSPVWEWPFGTGTKLTVL | |
| 141 | QVQLVQSGAEVKKPGASVKVSCKASGYAFTSYYMHWARQAPGQGLEWMGIINPS | MEDI-38-VH |
| | GGSASYAQKFQGRVTMTRDTSTSTVYMELSSLRSEDTAVYYCARGKYWMYDWGK | |
| | GTLVTVSS | |
| 142 | QSVLTQPPSVSAAPGQKVTISCSGGSSNIGNSYVSWYQQLPGTAPKLLIYDNNKRPS | MEDI-38-VL |
| | GIPDRFSGSKSGTSATLAITGLQTGDEADYFCGTWDTSTVWEWPFGTGTKLTVL | |
| 143 | QVQLVQSGAEVKKPGASVKVSCKASGYAFTSYYMHWARQAPGQGLEWMGIINPR | MEDI-39-VH |
| | GGSTSYAQKFQGRVTMTRDTSTSTVYMELSSLRSEDTAVYYCARGKYWMYDWGKG | |
| | TLVTVSS | |
| 144 | QSVLTQPPSVSAAPGQKVTISCSGGSSNIGNSYVSWYQQLPGTAPKLLIYDNNKRPS | MEDI-39-VL |
| | GIPDRFSGSKSGTSATLAITGLQTGDEADYYCGTWDTSTAWEWPFGTGTKLTVL | |
| 145 | QVQLVQSGAEVKKPGASVKVSCKASGYAFTSYYMHWARQAPGQGLEWMGIINPS | MEDI-40-VH |
| | GGSTSYAQKFQGRVTMTRDTSTSTVYMELSSLRSEDTAVYYCARGKYWMYDWGKG | |
| | TLVTVSS | |
| 146 | QSVLTQPPSVSAAPGQKVTISCSGGSSNIGNSYVSWYQQLPGTAPKLLIYDNNKRPS | MEDI-40-VL |
| | GIPDRFSGSKSGTSATLAITGLQTGDEADYYCGTWDSSTVWEWPFGTGTKLTVL | |
| 147 | QVQLVQSGAEVRKPGASVKVSCKASGYAFTSYYMHWARQAPGQGLEWMGIINPS | MEDI-41-VH |
| | GGSTSYAQKFQGRVTMTRDTSTSTVYMELSSLRPEDTAVYYCARGKYWMYDWGK | |
| | GTLVTVSG | |
| 148 | QSVLTQPPSVSAAPGQKVTISCSGGSTNIGNSYVSWYQRLPGTAPKLLIYDNNKRPP | MEDI-41-VL |
| | GIPDRFSGSKSGTSATLAITGLQTGDEADYYCGTWDTSTVWEWPFGTGTKLTVL | |
| 149 | QVQLVQSGAEVKKPGASVKVSCKASGYAFTSYYMHWARQAPGQGLEWVGIINPSG | MEDI-42-VH |
| | GSTSYAQKFQGRVTMTRDTSTSTVYMELSSLRSGDTAVYYCARGKYWMYDWGKGT | |
| | LVTVSS | |
| 150 | QAVLTQPPSVSAAPGQKVTISCSGGSSNIGNSYVSWYQRLPGAAPKLLIYDNNKRPS | MEDI-42-VL |
| | GIPDRFSGSKSGTSATLAITGLQTGDEADYYCGTWDTSTGWEWPFGTGTKLTVL | |
| 151 | QVQLVQSGAEVKKPGASVKVSCKASGYAFTSYYMHWVRQAPGQGLEWMGIINPR | MEDI-37GL-VH |
| | GGSTSYAQKFQGRVTMTRDTSTSTVYMELSSLRSEDTAVYYCARGKYWMYDWGKG | |
| | TLVTVSS | |
| 152 | QSVLTQPPSVSAAPGQKVTISCSGGGSSIGNSYVSWYQQLPGTAPKLLIYDNNKRPS | MEDI-37GL-VL |
| | GIPDRFSGSKSGTSATLGITGLQTGDEADYYCGTWDTSPVWEWPFGTGTKLTVL | |
| 153 | EVQLLESGGGLVQPGGSLRLSCAVSGFTFSNYAMSWVRQAPGKGLEWVSAISSGGG | AJOU-1-VH |
| | NIYYADSVKGRFTISRDNSKNTLYLQMNSLRAEDTAVYYCAKLRRYFDYWGQGTLVT | |
| | VSS | |
| 154 | EVQLLESGGGLVQPGGSLRLSCAASGFTFSDYAMSWVRQAPGKGLEWVSAISSGGS | AJOU-2-VH |
| | SIYYADSVKGRFTISRDNSKNTLHLQMNSLRAEDTAVYYCARGPQRSATAVFDYWG | |
| | QGTLVTVSS | |
| 155 | EVQLLESGGGLVQPGGSLRLSCAASGFTFSNYAMSWVRQAPGKGLEWVSWISPNS | AJOU-3-VH |
| | GNIYYADSVKGRFTISRDNSKNTLYLQMNSLRAEDTAVYYCARRPLSAAWSHSSYYN | |
| | AMDVWGQGTLVTVSS | |
| 156 | EVQLLESGGGLVQPGGSLRLSCAASGFTFSGYAMSWVRQAPGKGLEWVSLISHSGS | AJOU-4-VH |
| | NTYYADSVKGRFTISRDNSKNTLYLQMNSLRAEDTAVYYCARPHRAFDYWGQGTLV | |
| | TVSS | |
| 157 | EVQLLESGGGLVQPGGSLRLSCAASGFTFSNYAMSWVRQAPGKGLEWVSGISHGS | AJOU-5-VH |
| | GSIYYADSVKGRFTISRDNSKNTLYLQMNSLRAEDTAVYYCARPHRAFDYWGQGTLV | |
| | TVSS | |
| 158 | EVQLLESGGGLVQPGGSLRLSCAASGFTFSNYAMSWVRQAPGKGLEWVSGISHGN | AJOU-6-VH |
| | GSIYYADSVKGRFTISRDNSKNTLYLQMNSLRAEDTAVYYCAKTGRHFDYWGQGTLV | |
| | TVSS | |
| 159 | EVQLLESGGGLVQPGGSLRLSCAASGFTFSNYAMSWVRQAPGKGLEWVSSISPSGS | AJOU-7-VH |
| | SIYYADSVKGRFTISRDNSKNTLYLQMNSLRAEDTAVYYCARSYRAFDYWGQGTLVT | |
| | VSS | |
| 160 | EVQLLESGGGLVQPGGSLRLSCAASGFTFSNYAMSWVRQAPGKGLEWVSAISPSGG | AJOU-8-VH |
| | SIYYADSVKGRFTISRDNSKNTLYLQMNSLRAEDTAVYYCARAKRAFDYWGQGTLVT | |
| | VSS | |
| 161 | EVQLLESGGGLVQPGGSLRLSCAASGFTFSNYAMSWVRQAPGKGLEWVSAISPGSG | AJOU-9-VH |
| | STYYADSVKGRFTISRDNSKNTLYLQMNSLRAEDTAVYYCAKFRRHFDYWGQGTLVT | |
| | VSS | |
| 162 | EVQLLESGGGLVQPGGSLRLSCAASGFTFSNYAMSWVRQAPGKGLEWVSAISSGGG | AJOU-10-VH |
| | NIYYADSVKGRFTISRDNSKNTLYLQMNSLRAEDTAVYYCARVHRAFDYWGQGTLV | |
| | TVSS | |
| 163 | EVQLLESGGGLVQPGGSLRLSCAASGFTFSNYAMSWVRQAPGKGLEWVSAITSSGR | AJOU-69-VH |
| | SIYYADSVKGRFTISRDNSKNTLYLQMNSLRAEDTAVYYCARVHRAFDYWGQGTLVT | |
| | VSS | |
| 164 | EVQLLESGGGLVQPGGSLRLSCAASGFTFSNYAMSWVRQAPGKGLEWVSAITSSGA | AJOU-70-VH |
| | NIYYADSVKGRFTISRDNSKNTLYLQMNSLRAEDTAVYYCARVHRAFDYWGQGTLV | |
| | TVSS | |
| 165 | EVQLLESGGGLVQPGGSLRLSCAASGFTFSNYAMSWVRQAPGKGLEWVSAITSSGG | AJOU-71-VH |
| | NIYYADSVKGRFTISRDNSKNTLYLQMNSLRAEDTAVYYCARVHRAFDYWGQGTLV | |
| | TVSS | |
| 166 | EVQLLESGGGLVQPGGSLRLSCAASGFTFSNYAMSWVRQAPGKGLEWVSAITAGG | AJOU-72-VH |
| | GSIYYADSVKGRFTISRDNSKNTLYLQMNSLRAEDTAVYYCARVHRAFDYWGQGTLV | |
| | TVSS | |
| 167 | EVQLLESGGGLVQPGGSLRLSCAASGFTFSRHAMAWVRQAPGKGLEWVSAITSSGR | AJOU-83-VH |
| | SIYYADSVKGRFTISRDNSKNTLYLQMNSLRAEDTAVYYCARVHRAFDYWGQGTLVT | |
| | VSS | |
| 168 | QSVLTQPPSASGTPGQRVTISCSGSSSNIGNNYVNWYQQLPGTAPKLLIYDNSHRPS | AJOU-33-VL |
| | GVPDRFSGSKSGTSASLAISGLRSEDEADYYCGTWDASLSAYVFGGGTKLTVL | |
| 169 | QSVLTQPPSASGTPGQRVTISCSGSSSNIGNNNVSWYQQLPGTAPKLLIYANSKRPS | AJOU-34-VL |
| | GVPDRFSGSKSGTSASLAISGLRSEDEADYYCGSWDDSLSAYVFGGGTKLTVL | |
| 170 | QSVLTQPPSAPGTPGQRVTISCTGSSSNIGSNSVNWYQQLPGTAPKLLIYDDSHRPS | AJOU-35-VL |
| | GVPDRFSGSKSGTSASLAISGLRSEDEADYYCDAWDSSLSAYVFGGGTKLTVL | |
| 171 | QSVLIQPPSASGTPGQRVTLSCIGSSSNIGSNYVSWYQQLPGTAPKLLIYADSQRPSG | AJOU-36-VL |
| | VPDRFSGSKSGTSASLAISGLRSEDEADYYCGTWDDSLSGYVFGGGTKLTVL | |
| 172 | QSVLTQPPSASGTPGQRVTISCSSSSSNIGSNYVSWYQQLPGTAPKLLIYSDSHRPSG | AJOU-37-VL |
| | VPDRFSGSKSGTSASLAISGLRSEDEADYYCGSWDYSLSAYVFGGGTKLTVL | |
| 173 | QSVLTQPPSASGTPGQRVTISCTGSSSNIGNNTVSWYQQLPGTAPKLLIYDNSHRPS | AJOU-38-VL |
| | GVPDRFSGSKSGTSASLAISGLQSEDEADYYCGSWDYSLSAYVFGGGTKLTVL | |
| 174 | QSVLTQPPSASGTPGQRVTISCTGSSSNIGNNDVNWYQQLPGTAPKLLIYYDSQRPS | AJOU-39-VL |
| | GVPDRFSGSKSGTSASLAISGLRSEDEADYYCATWDASLSAYVFGGGTKLTVL | |
| 175 | QSVLTQPPSASGTPGQRVTISCSGSSSNIGSNAVNWYQQLPGTAPKLLIYYDNQRPS | AJOU-40-VL |
| | GVPDRFSGSKSGTSASLAISGLRSEDEADYYCGTWDDSLNGYVFGGGTKLTVL | |
| 176 | QSVLTQPPSASGTPGQRVTISCSGSSSNIGNNAVTWYQQLPGTAPKLLIYDDSHRPS | AJOU-41-VL |
| | GVPDRFSGSKSGTSASLAISGLRSEDEADYYCGSWDYSLSAYVFGGGTKLTVL | |
| 177 | QSVLTQPPSASGTPGQRVTISCSGSSSNIGSNTFNWYQQLPGTAPKLLIYADSHRPSG | AJOU-42-VL |
| | VPDRFSGSKSGTSASLAISGLRSEDEADYYCGTWDYSLSGYVLGGGTKLTVL | |
| 178 | QSVLTQPPSASGTPGQRVTISCSGSSSNIGSNTFNWYQQLPGTAPKLLIYADSHRPSG | AJOU-77-VL |
| | VPDRFSGSKSGTSASLAISGLRSEDEADYYCGTWDYSLSGYVLGGGTKLTVL | |
| 179 | QSVLTQPPSASGTPGQRVTISCSGSSSNIGSNTFNWYQQLPGTAPKLLIYADSHRPSG | AJOU-78-VL |
| | VPDRFSGSKSGTSASLAISGLRSEDEADYYCGTWDYSLRGYVLGGGTKLTVL | |
| 180 | QSVLTQPPSASGTPGQRVTISCSGSSSNIGSNTFNWYQQLPGTAPKLLIYADSHRPSG | AJOU-79-VL |
| | VPDRFSGSKSGTSASLAISGLRSEDEADYYCGYWDYSLSGYVLGGGTKLTVL | |
| 181 | QSVLTQPPSASGTPGQRVTISCSGSSSNIGSNTFNWYQQLPGTAPKLLIYADSHRPSG | AJOU-80-VL |
| | VPDRFSGSKSGTSASLAISGLRSEDEADYYCGTWDYSLSGYVLGGGTKLTVL | |
| 182 | QSVLTQPPSASGTPGQRVTISCSGSSANSRTDGFNWYQQLPGTAPKLLIYADSHRPS | AJOU-86-VL |
| | GVPDRFSGSKSGTSASLAISGLRSEDEADYYCGTWDYSLSGYVLGGGTKLTVLG | |
| 183 | QSVLTQPPSASGTPGQRVTISCSGSAQFGSRDNFNWYQQLPGTAPKLLIYADSHRPS | AJOU-87-VL |
| | GVPDRFSGSKSGTSASLAISGLRSEDEADYYCGTWDYSLSGYVLGGGTKLTVLG | |
| 184 | QSVLTQPPSASGTPGQRVTISCSGSTKQMHNYQFNWYQQLPGTAPKLLIYADSHRP | AJOU-88-VL |
| | SGVPDRFSGSKSGTSASLAISGLRSEDEADYYCGTWDYSLSGYVLGGGTKLTVLG | |
| 185 | QSVLTQPPSASGTPGQRVTISCSGSLLRGENLQFNWYQQLPGTAPKLLIYADSHRPS | AJOU-89-VL |
| | GVPDRFSGSKSGTSASLAISGLRSEDEADYYCGTWDYSLSGYVLGGGTKLTVLG | |
| 186 | QSVLTQPPSASGTPGQRVTISCSGSPLFPDSGSFNWYQQLPGTAPKLLIYADSHRPSG | AJOU-90-VL |
| | VPDRFSGSKSGTSASLAISGLRSEDEADYYCGTWDYSLSGYVLGGGTKLTVLG | |
| 187 | QSVLTQPPSASGTPGQRVTISCSGSAALDLSPSFNWYQQLPGTAPKLLIYADSHRPSG | AJOU-91-VL |
| | VPDRFSGSKSGTSASLAISGLRSEDEADYYCGTWDYSLSGYVLGGGTKLTVLG | |
| 188 | QVQLVQSGAEVKKPGASVKVSCKASGYTFTNYGISWVRQAPGQGLEWMGWISVY | REGN-VH-3 |
| | NGKTNYAQKLQGRVTMTTDTSTTTAYMEMRSLRSDDTAVYYCARGSGYDLDYWG | |
| | QGTLVSVSS | |
| 189 | EVQLVESGGGLVQPGGSLRLSCAASGFTFSSFWMTWVRQAPGKGLEWVANIKQD | REGN-VH-19 |
| | GSEKYYVDSVKGRFTISRDNAKNSLYLQMNSLRAEDTAVYYCARDPGRTMVRGGIRY | |
| | YYGMDVWGQGTTVTVSS | |
| 190 | EVKLAESGGGLVQPGGSLRLSCAASGFTFSSHWMNWVRQAPGKGLEWVANIKQD | REGN-VH-35 |
| | GSDKYYVDSVKGRFTISRDNAKNSLYLQLNSLIAEDTAVYYCARDRGVRPPRGAFDIW | |
| | GQGTMVTVSS | |
| 191 | QVQLVQSGAEVKKPGASVKVSCKASGYTFNSYGISWVRQAPGQGLEWMGWIRTY | REGN-VH-51 |
| | NGNTNYAQKLQGRVTMTTDTSTSTAYMELRSLRSDDTAVYYCARDEARIVVAGTTP | |
| | YYYGMDVWGQGTTVTVSS | |
| 192 | QVQLVESGGGLVQPGGSLRLSCAVSGFTISDHYMSWIRQAPGKGLEWISYISSSGSKI | REGN-VH-67 |
| | YYADSVKGRFTISRDNAKNSLFLQMNSLRAEDTAVYYCARTRQLVGDYWGQGTLVT | |
| | VSS | |
| 193 | EVQLVESGGGLVQPGRSLRLSCAASGFTFDNYAMHWVRQAPGKGLEWVSGIRWN | REGN-VH-83 |
| | SGSIGYADSVKGRFTISRDNAKNSLYLQMNSLRAEDTALYYCAKEGGYSGYRPGPEED | |
| | YWGQGTLVTVSS | |
| 194 | QVQLVQSGAEVKKPGASVKVSCKASGYTFTNYGISWVRQAPGQGLEWMGWISVY | REGN-VH-99 |
| | NGHTNYAQKLQGRVTMTTDTSTSTAYMELRSLRSDDTAVYYCARGSGYDFDSWGQ | |
| | GTLVTVSS | |
| 195 | QVQLVQSGAEVKKPGASVKVSCKASRYTFTSYDINWVRQATGQGLEWMGWMNP | REGN-VH-115 |
| | NSGNTGYAQKFQGRVTMTRNTSTSTAYMELSSLRSEDTAVYYCARVRRFFDYWGQ | |
| | GTLVTVSS | |
| 196 | QVQLVQSGPEVKKPGASVKVSCKASGYTFTNYGISWVRQAPGQGLEWMGWISVY | REGN-VH-147 |
| | NGNINYAQKLQGRVTMTTDTSTSTAYMDLRSLRSDDTAVYYCARGSGYDFDYWGQ | |
| | GTLVTVSS | |
| 197 | QVQLVQSGAEVKKPGASVKVSCKDSAYTFNRYGISWVRQAPGQGLEWMGWISAY | REGN-VH-163 |
| | TGNTVYAQKLQGRVTMTTDNSTSTAYMELRSLRSDDTAVYYCARDKSIFGVVRGFD | |
| | YWGQGTLVTVSS | |
| 198 | AIQMTQSPSSLSASVGDRVTITCRASQGIRNALGWYQQKPGKAPKLLIYAASSLQSG | REGN-VL-11 |
| | VPSRFSGSGSGTDFTLTFSSLQPEDFATYYCLQDFNYPYTFGQGTKLEIK | |
| 199 | DIQMTQSPSSVSASVGDRVTISCRASQGVSSWLAWYQQKPGNAPKLLISAASSIQSG | REGN-VL-27 |
| | VPSRFSGSGSGTDFTLTISSLQPEDFATYYCQQANSFPLTFGGGTKVEIK | |
| 200 | DIQMTQSPSSVSASVGDRVTITCRASQGISSWLAWYQQKPGKAPKLLIYAASSFQSG | REGN-VL-43 |
| | VPSRFSGSGSGTDFTLTISSLQPEDFATYFCQQANSFPLTFGGGTTVEIK | |
| 201 | DIQMTQSPSSVSASVGDRVTITCRASQDISIWLAWYQQSPGKAPKLLINVASRLQSG | REGN-VL-59 |
| | VPSRFSGSGSGTDFTLTINSLQPEDFVTYYCQQANSFPITFGQGTRLATK | |
| 202 | DIQLTQSPSFLSASVGDRVTITCWASQGISSYLAWYQQKPGKAPKLLIFAASTLQSGV | REGN-VL-75 |
| | PSRFSGSGSGTEFTLTISSLQPEDFATYYCQQLNSYPLTFGGGTKVEIR | |
| 203 | EIVMTQSPATLSVSPGERATLSCRASQSVNYNLAWYQHKPGQAPRLLIYGASTRATGI | REGN-VL-91 |
| | PARFSGSGSGTEFTLTISSLQSEDFAVYYCQQYNNWPLTFGGGTKVEIK | |
| 204 | AIQMTQSSSSLSASVGDRVTITCRASQAIRNALGWYQQKPGKAPKVLIYAASSLQSGI | REGN-VL-107 |
| | PSRFSGSGSGTDFTLTISSLQPEDFATYYCLQDYDYPYTFGQGTKLEIK | |
| 205 | DIQLTQSPSFLSASVGDRVTITCWASQGIISYLAWYQQKPGKAPKLLIYAASTLHSGVP | REGN-VL-123 |
| | SRFSGSGSGTEFTLTISSLQPEDFATYYCHQLKSYPITFGQGTRLEIK | |
| 206 | AIQMTQSPSSLSASVGDRVTITCRASQDIRNALGWYQQKPGKAPKLLIYAASSLQSG | REGN-VL-155 |
| | VPSRFSGSASGTDFTLTISSLQPEDFAAYYCLQDYNYPYTFGQGTKLEIK | |
| 207 | EIVMTQSPVTLSLSPGERATLPCRASQSVSSSLAWYQQKAGQSPRLLIYGASTRATGI | REGN-VL-171 |
| | PARFSGSGSGTEFTLTISNLQSEDFAVYYCQQYNNWPLTFGGGTKVEIK | |

1. A method for enhancing the efficacy, safety, and/or tolerability of a peanut allergen immunotherapy regimen in a subject having a peanut allergy, the method comprising administering to the subject one or more doses of an interleukin-4 receptor (IL-4R) antagonist in combination with the immunotherapy regimen, wherein at least one dose of the IL-4R antagonist is administered prior to the start of the immunotherapy regimen, and wherein the IL-4R antagonist is an anti-IL-4R antibody, or an antigen-binding fragment thereof, that comprises the heavy chain complementarity determining regions (HCDRs) of a heavy chain variable region (HCVR) comprising the amino acid sequence of SEQ ID NO:1 and the light chain complementarity determining regions (LCDRs) of a light chain variable region (LCVR) comprising the amino acid sequence of SEQ ID NO:2.

2. The method of claim 1, wherein the immunotherapy regimen is an oral immunotherapy (OIT) regimen.

3. The method of claim 1, wherein the peanut allergen is a composition comprising peanut flour.

4. The method of claim 1, wherein the immunotherapy regimen comprises an up-dosing phase followed by a maintenance phase, wherein the up-dosing phase comprises administering increasing doses of the peanut allergen over a period of at least 24 weeks and wherein the maintenance phase comprises administering one or more maintenance doses of the peanut allergen at the highest dose administered during the up-dosing phase.

5. The method of claim 4, wherein the up-dosing phase comprises an initial dose escalation day (IDED) regimen followed by increasing dose escalations every two weeks.

6. The method of claim 4, wherein the up-dosing phase comprises up-dosing from an initial dose of 0.5 mg peanut protein to a dose of 300 mg peanut protein and wherein the maintenance phase comprises administering one or more maintenance doses of 300 mg peanut protein.

7. The method of claim 1, wherein the subject is aged ≥6 years old to <18 years old.

8. The method of claim 1, wherein the subject has one or more of the following baseline characteristics:
(a) a clinical history of allergy to peanuts or peanut-containing foods;
(b) experiences dose-limiting symptoms at or before a challenge dose of 100 mg peanut protein, or at a cumulative dose of ≤144 mg of peanut protein, in a double-blind, placebo-controlled food challenge (DBPCFC);
(c) has a serum IgE to peanut of ≥10 kUA/L; or
(d) has a skin prick test (SPT) to peanut ≥8 mm.

9. The method of claim 1, wherein the subject has concomitant atopic dermatitis, asthma, eosinophilic esophagitis, and/or multiple food allergies.

10. The method of claim 1, wherein the IL-4R antagonist is administered at a dose of about 50 mg to about 600 mg.

11. The method of claim 1, wherein the IL-4R antagonist is administered as an initial dose followed by one or more secondary doses, wherein each secondary dose is administered 1 to 4 weeks after the immediately preceding dose.

12. The method of claim 11, wherein each secondary dose of the IL-4R antagonist is administered two weeks after the immediately preceding dose.

13. The method of claim 1, wherein the IL-4R antagonist is administered subcutaneously or intravenously.

14. The method of claim 1, wherein the IL-4R antagonist is subcutaneously administered at an initial dose followed by one or more secondary doses, wherein each secondary dose is administered 1 to 4 weeks after the immediately preceding dose, and wherein:
(i) for a subject weighing <30 kg, the initial dose of the IL-4R antagonist is 200 mg and each secondary dose is 100 mg; or
(ii) for a subject having a body weight of ≥30 kg to <60 kg, the initial dose of the IL-4R antagonist is 400 mg and each secondary dose is 200 mg; or
(iii) for a subject having a body weight of ≥60 kg, the initial dose of the IL-4R antagonist is 600 mg and each secondary dose is 300 mg.

15. The method of claim 14, wherein the subject has a body weight of <30 kg and the TL-4R antagonist is administered at an initial dose of 200 mg followed by one or more secondary doses of 100 mg every two weeks (Q2W).

16. The method of claim 14, wherein the subject has a body weight of ≥30 kg to <60 mg and the IL-4R antagonist is administered at an initial dose of 400 mg followed by one or more secondary doses of 200 mg every two weeks (Q2W).

17. The method of claim 14, wherein the subject has a body weight of ≥60 mg and the TL-4R antagonist is administered at an initial dose of 600 mg followed by one or more secondary doses of 300 mg every two weeks (Q2W).

18. The method of claim 11, wherein the initial dose of the IL-4R antagonist is administered at least two weeks before the start of the immunotherapy regimen.

19. The method of claim 18, wherein the IL-4R antagonist is administered for at least four weeks before the start of the immunotherapy regimen.

20. The method of claim 1, wherein administration of the IL-4R antagonist:
(i) increases the cumulative tolerated dose of peanut protein as measured by a DBPCFC; and/or
(ii) decreases the frequency and/or severity of peanut allergic symptoms, gastrointestinal symptoms, and/or itching symptoms.

21. The method of claim 1, wherein the anti-IL-4R antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO:3; the HCDR2 comprises the amino acid sequence of SEQ ID NO:4; the HCDR3 comprises the amino acid sequence of SEQ ID NO:5; the LCDR1 comprises the amino acid sequence of SEQ ID NO:6; the LCDR2 comprises the amino acid sequence of SEQ ID NO:7; and the LCDR3 comprises the amino acid sequence of SEQ ID NO:8.

22. The method of claim 1, wherein the anti-IL-4R antibody or antigen-binding fragment thereof comprises a HCVR comprising the amino acid sequence of SEQ ID NO:1 and comprises a LCVR comprising the amino acid sequence of SEQ ID NO:2.

23. The method of claim 1, wherein the anti-IL-4R antibody comprises a heavy chain comprising the amino acid sequence of SEQ ID NO:9 and a light chain comprising the amino acid sequence of SEQ ID NO:10.

24. The method of claim 1, wherein the IL-4R antagonist is dupilumab.

25. The method of claim 1, wherein the IL-4R antagonist is contained in a container selected from the group consisting of a glass vial, a syringe, a pre-filled syringe, a pen delivery device, and an autoinjector.

26. The method of claim 25, wherein the IL-4R antagonist is contained in a pre-filled syringe.

27. The method of claim 26, wherein the pre-filled syringe is a single-dose pre-filled syringe.

28. The method of claim 25, wherein the TL-4R antagonist is contained in an autoinjector.

29. The method of claim 25, wherein the TL-4R antagonist is contained in a pen delivery device.


SEQUENCE LISTING (207 sequences)

SEQ ID NO: 1 - protein, 124 aa, Artificial Sequence
  Description of Artificial Sequence Synthetic polypeptide
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 2 - protein, 112 aa, Artificial Sequence [sequence omitted]

SEQ ID NO: 3 - protein, 8 aa, Artificial Sequence
  Description of Artificial Sequence Synthetic peptide
GFTFRDYA

SEQ ID NO: 4 - protein, 8 aa, Artificial Sequence
  Description of Artificial Sequence Synthetic peptide
ISGSGGNT

SEQ ID NO: 5 - protein, 18 aa, Artificial Sequence
  Description of Artificial Sequence Synthetic peptide
AKDRLSITIRPRYYGLDV

SEQ ID NO: 6 - protein, 11 aa, Artificial Sequence
  Description of Artificial Sequence Synthetic peptide
QSLLYSIGYNY

SEQ ID NO: 7 - protein, 3 aa, Artificial Sequence
  Description of Artificial Sequence Synthetic peptide
LGS

SEQ ID NO: 8 - protein, 9 aa, Artificial Sequence
  Description of Artificial Sequence Synthetic peptide
MQALQTPYT

SEQ ID NO: 9 - protein, 452 aa, Artificial Sequence
  Description of Artificial Sequence Synthetic polypeptide
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 219 aa, Artificial Sequence
  Description of Artificial Sequence Synthetic polypeptide
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 207 aa, Homo sapiens
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQH

SEQ ID NO: 12 - protein, 109 aa, Artificial Sequence
  Description of Artificial Sequence Synthetic polypeptide
EIVLTQSPGTLSLSPGERATLSCRASQSVSNSYLAWYQQKPGQAPRLLIFGASSRATGIPDRFSGSGSGTDFTLTISRLEPEDFAVYYCQQYGSSPPWTFGQGTKVEIK

SEQ ID NO: 13 - protein, 109 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPWTFGQGTKVEIK

SEQ ID NO: 14 - protein, 109 aa, Artificial Sequence
  Description of Artificial Sequence Synthetic polypeptide
EIVLTQSPGTLSLSPGERATLSCRASQSVSSSYLAWYQQKPGQAPRLLIFGASSRAPGIPDRFSGSGSGTDFTLTISRLEPEDFAVYYCQQYGSSPPWTFGQGTKVEIK

SEQ ID NO: 15 - protein, 109 aa, Artificial Sequence
  Description of Artificial Sequence Synthetic polypeptide
EIVLTQSPGTLSLSPGERATLSCRASQSVSNSYLAWYQQKPGQAPRLLIYGASSRATGIPDRFSGSGSGTDFTLTISRLEPEDFAVYYCQQYGSSPPWTFGQGTKVEIK

SEQ ID NO: 16 - protein, 109 aa, Artificial Sequence
  Description of Artificial Sequence Synthetic polypeptide
EIVLTQSPGTLSLSPGERATLSCRASQSVSNSYLAWYQQKPGQAPRLLIFGASSRAPGIPDRFSGSGSGTDFTLTISRLEPEDFAVYYCQQYGSSPPWTFGQGTKVEIK

SEQ ID NO: 17 - protein, 109 aa, Artificial Sequence
  Description of Artificial Sequence Synthetic polypeptide
EIVLTQSPGTLSLSPGERATLSCRASQSVSSSYLAWYQQKPGQAPRLLIYGASSRAPGIPDRFSGSGSGTDFTLTISRLEPEDFAVYYCQQYGSSPPWTFGQGTKVEIK

SEQ ID NO: 18 - protein, 109 aa, Artificial Sequence
  Description of Artificial Sequence Synthetic polypeptide
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSPPWTFGQGTKVEIK

SEQ ID NO: 19 - protein, 109 aa, Artificial Sequence
  Description of Artificial Sequence Synthetic polypeptide
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSAGWTFGQGTKVEIK

SEQ ID NO: 20 - protein, 109 aa, Artificial Sequence
  Description of Artificial Sequence Synthetic polypeptide
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSAGWTFGQGTKVEIK

SEQ ID NO: 21 - protein, 109 aa, Artificial Sequence
  Description of Artificial Sequence Synthetic polypeptide
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSPPWTFGQGTKVEIK

SEQ ID NO: 22 - protein, 109 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSPPWTFGQGTKVEIK

SEQ ID NO: 23 - protein, 109 aa, Artificial Sequence
  Description of Artificial Sequence Synthetic polypeptide
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSPPWTFGQGTKVEIK

SEQ ID NO: 24 - protein, 109 aa, Artificial Sequence
  Description of Artificial Sequence Synthetic polypeptide
EIVLTQSPGTLSLSPGERATLSCRASQSVSSSYLAWYQQKPGQAPRLLIYGASSRAPGIPDRFSGSGSGTDFTLTISRLEPEDFAVYYCQQYDHSAGWTFGQGTKVEIK

SEQ ID NO: 25 - protein, 109 aa, Artificial Sequence
  Description of Artificial Sequence Synthetic polypeptide
EIVLTQSPGTLSLSPGERATLSCRASQSVSNSYLAWYQQKPGQAPRLLIFGASSRAPGIPDRFSGSGSGTDFTLTISRLEPEDFAVYYCQQYDHSAGWTFGQGTKVEIK

SEQ ID NO: 26 - protein, 109 aa, Artificial Sequence
  Description of Artificial Sequence Synthetic polypeptide
EIVLTQSPGTLSLSPGERATLSCRASQSVSNSYLAWYQQKPGQAPRLLIYGASSRATGIPDRFSGSGSGTDFTLTISRLEPEDFAVYYCQQYDHSAGWTFGQGTKVEIK

SEQ ID NO: 27 - protein, 109 aa, Artificial Sequence
  Description of Artificial Sequence Synthetic polypeptide
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSAGWTFGQGTKVEIK

SEQ ID NO: 28 - protein, 109 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSAGWTFGQGTKVEIK

SEQ ID NO: 29 - protein, 109 aa, Artificial Sequence
  Description of Artificial Sequence Synthetic polypeptide
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSAGWTFGQGTKVEIK

SEQ ID NO: 30 - protein, 109 aa, Artificial Sequence
  Description of Artificial Sequence Synthetic polypeptide
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSPPWTFGQGTKVEIK

SEQ ID NO: 31 - protein, 109 aa, Artificial Sequence
  Description of Artificial Sequence Synthetic polypeptide
EIVLTQSPGTLSLSPGERATLSCRASQSVSNSYLAWYQQKPGQAPRLLIYGASSRAPGIPDRFSGSGSGTDFTLTISRLEPEDFAVYYCQQYDHSAGWTFGQGTKVEIK

SEQ ID NO: 32 - protein, 115 aa, Artificial Sequence
  Description of Artificial Sequence Synthetic polypeptide
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 33 - protein, 115 aa, Artificial Sequence
  Description of Artificial Sequence Synthetic polypeptide
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 34 - protein, 115 aa, Artificial Sequence
  Description of Artificial Sequence Synthetic polypeptide
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 35 - protein, 115 aa, Artificial Sequence
  Description of Artificial Sequence Synthetic polypeptide
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFDYWGQGTLVTVSS

SEQ ID NO: 36 - protein, 115 aa, Artificial Sequence
  Description of Artificial Sequence Synthetic polypeptide
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFDYWGQGTLVTVSS

SEQ ID NO: 37 - protein, 115 aa, Artificial Sequence
  Description of Artificial Sequence Synthetic polypeptide
EVQLVESGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 38 - protein, 115 aa, Artificial Sequence
  Description of Artificial Sequence Synthetic polypeptide
EVQLVESGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 39 - protein, 115 aa, Artificial Sequence
  Description of Artificial Sequence Synthetic polypeptide
EVQLVQSGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 40 - protein, 115 aa, Artificial Sequence
  Description of Artificial Sequence Synthetic polypeptide
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 41 - protein, 115 aa, Artificial Sequence
  Description of Artificial Sequence Synthetic polypeptide
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 42 - protein, 115 aa, Artificial Sequence
  Description of Artificial Sequence Synthetic polypeptide
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 43 - protein, 115 aa, Artificial Sequence
  Description of Artificial Sequence Synthetic polypeptide
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 44 - protein, 115 aa, Artificial Sequence
  Description of Artificial Sequence Synthetic polypeptide
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 45 - protein, 115 aa, Artificial Sequence
  Description of Artificial Sequence Synthetic polypeptide
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 46 - protein, 115 aa, Artificial Sequence
  Description of Artificial Sequence Synthetic polypeptide
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 47 - protein, 115 aa, Artificial Sequence
  Description of Artificial Sequence Synthetic polypeptide
EVQLVQSGGGLVHPGRSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 48 - protein, 115 aa, Artificial Sequence
  Description of Artificial Sequence Synthetic polypeptide
EVQLVQSGGGLVHPGGSLRLTCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 49 - protein, 115 aa, Artificial Sequence
  Description of Artificial Sequence Synthetic polypeptide
EVQLVQSGGGLVHPGGSLRLSCAGSGFTFSRNAMHWVRQAPGKGLEWVSGIGTGGATNYADSVKGRFTISRDNAKNSLYLQMNSLRAEDMAVYYCARGRYYFDYWGQGTLVTVSS

SEQ ID NO: 50 - protein, 115 aa, Artificial Sequence
  Description of Artificial Sequence Synthetic polypeptide
EVQLVQSGGGLVHPGGSLRLSCAGSGFTFSRNAMFWVRQAPGEGLEWVSGIGTGGATNYADSVKGRFTISRDNAKNSLYLQMNSLRAEDMAVYYCARGRYYFDYWGQGTLVTVSS

SEQ ID NO: 51 - protein, 115 aa, Artificial Sequence
  Description of Artificial Sequence Synthetic polypeptide
EVQLVQSGGGLVHPGGSLRLSCAGSGFTFSRNAMFWVRQAPGKGLEWVSGIGTGGATNYADSVKGRFTISRDEAKNSLYLQMNSLRAEDMAVYYCARGRYYFDYWGQGTLVTVSS

SEQ ID NO: 52 - protein, 115 aa, Artificial Sequence
  Description of Artificial Sequence Synthetic polypeptide
EVQLVQSGGGLVHPGGSLRLSCAGSGFTFSRNAMFWVRQAPGKGLEWVSGIGTGGATNYADSVKGRFTISRDNAKNSLYLQMNSLRAGDMAVYYCARGRYYFDYWGQGTLVTVSS

SEQ ID NO: 53 - protein, 115 aa, Artificial Sequence
  Description of Artificial Sequence Synthetic polypeptide
EVQLVQSGGGLVHPGGSLRLSCAGSGFTFDDYAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 54 - protein, 115 aa, Artificial Sequence
  Description of Artificial Sequence Synthetic polypeptide
EVQLVQSGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 55 - protein, 115 aa, Artificial Sequence
  Description of Artificial Sequence Synthetic polypeptide
EVQLVESGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 56 - protein, 115 aa, Artificial Sequence
  Description of Artificial Sequence Synthetic polypeptide
EVQLVESGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 57 - protein, 115 aa, Artificial Sequence
  Description of Artificial Sequence Synthetic polypeptide
EVQLVESGGGLVQPGGSLRLSCAASGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 58 - protein, 115 aa, Artificial Sequence
  Description of Artificial Sequence Synthetic polypeptide
EVQLVESGGGLVQPGGSLRLSCAASGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 59 - protein, 115 aa, Artificial Sequence
  Description of Artificial Sequence Synthetic polypeptide
EVQLVQSGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 60 - protein, 115 aa, Artificial Sequence
  Description of Artificial Sequence Synthetic polypeptide
EVQLVQSGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 61 - protein, 115 aa, Artificial Sequence
  Description of Artificial Sequence Synthetic polypeptide
EVQLVESGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 62 - protein, 115 aa, Artificial Sequence
  Description of Artificial Sequence Synthetic polypeptide
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 63 - protein, 115 aa, Artificial Sequence
  Description of Artificial Sequence Synthetic polypeptide
EVQLVESGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 64 - protein, 115 aa, Artificial Sequence
  Description of Artificial Sequence Synthetic polypeptide
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 65 - protein, 115 aa, Artificial Sequence
  Description of Artificial Sequence Synthetic polypeptide
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 66 - protein, 115 aa, Artificial Sequence
  Description of Artificial Sequence Synthetic polypeptide
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 67 - protein, 116 aa, Artificial Sequence
  Description of Artificial Sequence Synthetic polypeptide
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLDYWGKGTLVTVSS

SEQ ID NO: 68 - protein, 111 aa, Artificial Sequence
  Description of Artificial Sequence Synthetic polypeptide
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSLSANYVFGTGTKLTVL

SEQ ID NO: 69 - protein, 116 aa, Artificial Sequence
  Description of Artificial Sequence Synthetic polypeptide
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYNWGKGTLVTVSS

SEQ ID NO: 70 - protein, 111 aa, Artificial Sequence
  Description of Artificial Sequence Synthetic polypeptide
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSQPPNPLFGTGTKLTVL

SEQ ID NO: 71 - protein, 116 aa, Artificial Sequence
  Description of Artificial Sequence Synthetic polypeptide
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKLLKNPWGKGTLVTVSS

SEQ ID NO: 72 - protein, 111 aa, Artificial Sequence
  Description of Artificial Sequence Synthetic polypeptide
QSVLTQPPSVSAAPGQKVTISCSGGSSNIGNSYVSWYQQLPGTAPKLLIYDNNKRPSGIPDRFSGSKSGTSATLAITGLQTGDEADYYCGTWFGTPASNYVFGTGTKLTVL

SEQ ID NO: 73 - protein, 116 aa, Artificial Sequence; identical to SEQ ID NO: 69
  Description of Artificial Sequence Synthetic polypeptide

SEQ ID NO: 74 - protein, 111 aa, Artificial Sequence
  Description of Artificial Sequence Synthetic polypeptide
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSSPPQPIFGTGTKLTVL

SEQ ID NO: 75 - protein, 116 aa, Artificial Sequence
  Description of Artificial Sequence Synthetic polypeptide
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYDWGKGTLVTVSS

SEQ ID NO: 76 - protein, 111 aa, Artificial Sequence; identical to SEQ ID NO: 74
  Description of Artificial Sequence Synthetic polypeptide

SEQ ID NO: 77 - protein, 116 aa, Artificial Sequence
  Description of Artificial Sequence Synthetic polypeptide
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 78 - protein, 111 aa, Artificial Sequence
  Description of Artificial Sequence Synthetic polypeptide
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTYHPIFGTGTKLTVL

SEQ ID NO: 79 - protein, 116 aa, Artificial Sequence
  Description of Artificial Sequence Synthetic polypeptide
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQYWGKGTLVTVSS

SEQ ID NO: 80 - protein, 111 aa, Artificial Sequence; identical to SEQ ID NO: 74
  Description of Artificial Sequence Synthetic polypeptide

SEQ ID NO: 81 - protein, 116 aa, Artificial Sequence; identical to SEQ ID NO: 79
  Description of Artificial Sequence Synthetic polypeptide

SEQ ID NO: 82 - protein, 111 aa, Artificial Sequence; identical to SEQ ID NO: 78
  Description of Artificial Sequence Synthetic polypeptide

SEQ ID NO: 83 - protein, 116 aa, Artificial Sequence; identical to SEQ ID NO: 69
  Description of Artificial Sequence Synthetic polypeptide

SEQ ID NO: 84 - protein, 111 aa, Artificial Sequence
  Description of Artificial Sequence Synthetic polypeptide
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTMYPLFGTGTKLTVL

SEQ ID NO: 85 - protein, 116 aa, Artificial Sequence; identical to SEQ ID NO: 75
  Description of Artificial Sequence Synthetic polypeptide

SEQ ID NO: 86 - protein, 111 aa, Artificial Sequence
  Description of Artificial Sequence Synthetic polypeptide
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVLTPIFGTGTKLTVL

SEQ ID NO: 87 - protein, 116 aa, Artificial Sequence
  Description of Artificial Sequence Synthetic polypeptide
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWFYDWGKGTLVTVSS

SEQ ID NO: 88 - protein, 111 aa, Artificial Sequence
  Description of Artificial Sequence Synthetic polypeptide
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPSMIPLFGTGTKLTVL

SEQ ID NO: 89 - protein, 116 aa, Artificial Sequence; identical to SEQ ID NO: 87
  Description of Artificial Sequence Synthetic polypeptide

SEQ ID NO: 90 - protein, 111 aa, Artificial Sequence; identical to SEQ ID NO: 84
  Description of Artificial Sequence Synthetic polypeptide

SEQ ID NO: 91 - protein, 116 aa, Artificial Sequence; identical to SEQ ID NO: 75
  Description of Artificial Sequence Synthetic polypeptide

SEQ ID NO: 92 - protein, 111 aa, Artificial Sequence
  Description of Artificial Sequence Synthetic polypeptide
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTLQPLFGTGTKLTVL

SEQ ID NO: 93 - protein, 116 aa, Artificial Sequence; identical to SEQ ID NO: 69
  Description of Artificial Sequence Synthetic polypeptide

SEQ ID NO: 94 - protein, 111 aa, Artificial Sequence
  Description of Artificial Sequence Synthetic polypeptide
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPPTKPLFGTGTKLTVL

SEQ ID NO: 95 - protein, 116 aa, Artificial Sequence; identical to SEQ ID NO: 69
  Description of Artificial Sequence Synthetic polypeptide

SEQ ID NO: 96 - protein, 111 aa, Artificial Sequence
  Description of Artificial Sequence Synthetic polypeptide
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTHRHPLFGTGTKLTVL

SEQ ID NO: 97 - protein, 116 aa, Artificial Sequence; identical to SEQ ID NO: 69
  Description of Artificial Sequence Synthetic polypeptide

SEQ ID NO: 98 - protein, 111 aa, Artificial Sequence; identical to SEQ ID NO: 78
  Description of Artificial Sequence Synthetic polypeptide

SEQ ID NO: 99 - protein, 116 aa, Artificial Sequence
  Description of Artificial Sequence Synthetic polypeptide
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQHWGKGTLVTVSS

SEQ ID NO: 100 - protein, 111 aa, Artificial Sequence
  Description of Artificial Sequence Synthetic polypeptide
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVDRPIFGTGTKLTVL

SEQ ID NO: 101 - protein, 116 aa, Artificial Sequence; identical to SEQ ID NO: 99
  Description of Artificial Sequence Synthetic polypeptide

SEQ ID NO: 102 - protein, 111 aa, Artificial Sequence
  Description of Artificial Sequence Synthetic polypeptide
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTPMPVFGTGTKLTVL

SEQ ID NO: 103 - protein, 116 aa, Artificial Sequence; identical to SEQ ID NO: 99
  Description of Artificial Sequence Synthetic polypeptide

SEQ ID NO: 104 - protein, 111 aa, Artificial Sequence; identical to SEQ ID NO: 78
  Description of Artificial Sequence Synthetic polypeptide

SEQ ID NO: 105 - protein, 116 aa, Artificial Sequence; identical to SEQ ID NO: 77
  Description of Artificial Sequence Synthetic polypeptide

SEQ ID NO: 106 - protein, 111 aa, Artificial Sequence
  Description of Artificial Sequence Synthetic polypeptide
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVWEWPFGTGTKLTVL

SEQ ID NO: 107 - protein, 116 aa, Artificial Sequence
  Description of Artificial Sequence Synthetic polypeptide
QVQLVQSGAEVKKPGASVKVSCKASGYAFTSYYMHWARQAPGQGLEWMGIINPSGGSASYAQKFQGRVTMTRDTSTSTVYMELSSLRSEDTAVYYCARGKYWMYDWGKGTLVTVSS

SEQ ID NO: 108 - protein, 111 aa, Artificial Sequence
  Description of Artificial Sequence Synthetic polypeptide
QSVLTQPPSVSAAPGQKVTISCSGGSSNIGNSYVSWYQQLPGTAPKLLIYDNNKRPSGIPDRFSGSKSGTSATLAITGLQTGDEAVYFCGTWDTSTVWEWPFGTGTKLTVL

SEQ ID NO: 109 - protein, 116 aa, Artificial Sequence; identical to SEQ ID NO: 77
  Description of Artificial Sequence Synthetic polypeptide

SEQ ID NO: 110 - protein, 111 aa, Artificial Sequence
  Description of Artificial Sequence Synthetic polypeptide
QPVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 111 - protein, 116 aa, Artificial Sequence
  Description of Artificial Sequence Synthetic polypeptide
QVQLVQSGAEVRKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 112 - protein, 111 aa, Artificial Sequence
  Description of Artificial Sequence Synthetic polypeptide
QSVLTQPPSVSAAPGQKVTISCSGGSSNIGNNYVSWYQQLPGTAPKLLIYDNNKRPPGIPDRFSGSKSGTSATLAITGLQTGDEADYYCGTWDTSTVWEWPFGTGTKLTVL

SEQ ID NO: 113 - protein, 116 aa, Artificial Sequence
  Description of Artificial Sequence Synthetic polypeptide
QVQLVQSGAEVKKPGASVKVSCKASGYAFTSYYMHWARQAPGQGLEWMGIINPRGGSTSYAQKFQGRVTMTRDTSTSTVYMELSSLRSEDTAVYYCARGKYWMYDWGKGTLVTVSS

SEQ ID NO: 114 - protein, 111 aa, Artificial Sequence
  Description of Artificial Sequence Synthetic polypeptide
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 115 - protein, 116 aa, Artificial Sequence
  Description of Artificial Sequence Synthetic polypeptide
QVQLVQSGAEVKKPGASVKVSCKASGYAFTSYYMHWARQAPGQGLEWMGIINPRGGSASYAQKFQGRVSMTRDTSTSTVYMELSSLRSEDTAVYYCARGKYWMYDWGKGTLVTVSS

SEQ ID NO: 116 - protein, 111 aa, Artificial Sequence
  Description of Artificial Sequence Synthetic polypeptide
QSVLTQPPSVSAAPGQKVTISCSGGSSNIGNSYVSWYQQLPGTAPKLLIYDNNKRPSGIPDRFSGSKSGTTATLAITGLQTGDEADYYCGTWVTSTVWEWPFGTGTKLTVL

SEQ ID NO: 117 - protein, 116 aa, Artificial Sequence; identical to SEQ ID NO: 77
  Description of Artificial Sequence Synthetic polypeptide

SEQ ID NO: 118 - protein, 111 aa, Artificial Sequence; identical to SEQ ID NO: 114
  Description of Artificial Sequence Synthetic polypeptide

SEQ ID NO: 119 - protein, 116 aa, Artificial Sequence
  Description of Artificial Sequence Synthetic polypeptide
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTQVTVSS

SEQ ID NO: 120 - protein, 111 aa, Artificial Sequence
  Description of Artificial Sequence Synthetic polypeptide
QSVLTQPPLVSAAPGQKVTISCSGGSSNIGNSYVSWYQRLPGTAPKLLIYDNNKRPSGIPDRFSGSKSGTSATLAITGLQTGDEADYYCGTWDTSTVWEWPFGTGTKLTVL

SEQ ID NO: 121 - protein, 116 aa, Artificial Sequence
  Description of Artificial Sequence Synthetic polypeptide
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGNGTLVTVSS

SEQ ID NO: 122 - protein, 111 aa, Artificial Sequence
  Description of Artificial Sequence Synthetic polypeptide
LPVLTQPPSVSAAPGQKVTISCSGGSSSIGNSYVSWYQQLPGAAPKLLIYDNNKRPSGIPDRFSGFRSGTSATLAITGLQTGDEADYYCGTWDTSPVWEWPFGTGTKLTVL

SEQ ID NO: 123 - protein, 116 aa, Artificial Sequence
  Description of Artificial Sequence Synthetic polypeptide
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTRVTVSS

SEQ ID NO: 124 - protein, 111 aa, Artificial Sequence
  Description of Artificial Sequence Synthetic polypeptide
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVWEWPFGTGTKLTVL

SEQ ID NO: 125 - protein, 116 aa, Artificial Sequence; identical to SEQ ID NO: 77
  Description of Artificial Sequence Synthetic polypeptide

SEQ ID NO: 126 - protein, 111 aa, Artificial Sequence
  Description of Artificial Sequence Synthetic polypeptide
QSVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTVWEWPFGTGTKLTVL

SEQ ID NO: 127 - protein, 116 aa, Artificial Sequence; identical to SEQ ID NO: 77
  Description of Artificial Sequence Synthetic polypeptide

SEQ ID NO: 128 - protein, 111 aa, Artificial Sequence
  Description of Artificial Sequence Synthetic polypeptide
QSVLTQPPSVSAAPGQKVTISCSGGSSSIGNSYVSWYQQLPGTAPKLLIYDNNKRPSGIPDRFSGSKSGTSATLAITGLQTGDEADYYCGTWATSPVWEWPFGTGTKLTVL

SEQ ID NO: 129 - protein, 116 aa, Artificial Sequence; identical to SEQ ID NO: 77
  Description of Artificial Sequence Synthetic polypeptide

SEQ ID NO: 130 - protein, 111 aa, Artificial Sequence
  Description of Artificial Sequence Synthetic polypeptide
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTAWEWPFGTGTKLTVL

SEQ ID NO: 131 - protein, 116 aa, Artificial Sequence
  Description of Artificial Sequence Synthetic polypeptide
QVQLVQSGAEEKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 132 - protein, 111 aa, Artificial Sequence
  Description of Artificial Sequence Synthetic polypeptide
QSALTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 133 - protein, 116 aa, Artificial Sequence
  Description of Artificial Sequence Synthetic polypeptide
QVQLVQSGAEVKKPGASVKVSCKASGYAFTSYYMHWARQAPGQGLEWMGIINPSGGSTSYAQKFQGRVSMTRDTSTSTVYMELSSLRSEDTAVYYCARGKYWMYDWGKGTLVTVSS

SEQ ID NO: 134 - protein, 111 aa, Artificial Sequence; identical to SEQ ID NO: 114
  Description of Artificial Sequence Synthetic polypeptide

SEQ ID NO: 135 - protein, 116 aa, Artificial Sequence; identical to SEQ ID NO: 77
  Description of Artificial Sequence Synthetic polypeptide

SEQ ID NO: 136 - protein, 111 aa, Artificial Sequence; identical to SEQ ID NO: 124
  Description of Artificial Sequence Synthetic polypeptide

SEQ ID NO: 137 - protein, 116 aa, Artificial Sequence; identical to SEQ ID NO: 107
  Description of Artificial Sequence Synthetic polypeptide

SEQ ID NO: 138 - protein, 111 aa, Artificial Sequence
  Description of Artificial Sequence Synthetic polypeptide
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDSSTVWEWPFGTGTKLTVL

SEQ ID NO: 139 - protein, 116 aa, Artificial Sequence
  Description of Artificial Sequence Synthetic polypeptide
QVQLVQSGAEVKKPGASVKVSCKASGYAFTSYYMHWARQAPGQGLEWMGIINPRGGSTSYAQKFQGRVAMTRDTSTSTVYMELSSLRPEDTAVYYCARGKYWMYDWGKGTLVTVSS

SEQ ID NO: 140 - protein, 111 aa, Artificial Sequence
  Description of Artificial Sequence Synthetic polypeptide
QSVLTQPPSVSAAPGQKVTISCSGGGSSIGNSYVSWYQQLPGTAPKLLIYDNNKRPSGVPDRFSGSKSGTSATLAITGLQTGDEADYYCGTWDTSPVWEWPFGTGTKLTVL

SEQ ID NO: 141 - protein, 116 aa, Artificial Sequence; identical to SEQ ID NO: 107
  Description of Artificial Sequence Synthetic polypeptide

SEQ ID NO: 142 - protein, 111 aa, Artificial Sequence; identical to SEQ ID NO: 114
  Description of Artificial Sequence Synthetic polypeptide

SEQ ID NO: 143 - protein, 116 aa, Artificial Sequence; identical to SEQ ID NO: 113
  Description of Artificial Sequence Synthetic polypeptide

SEQ ID NO: 144 - protein, 111 aa, Artificial Sequence
  Description of Artificial Sequence Synthetic polypeptide
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTAWEWPFGTGTKLTVL

SEQ ID NO: 145 - protein, 116 aa, Artificial Sequence; identical to SEQ ID NO: 77
  Description of Artificial Sequence Synthetic polypeptide

SEQ ID NO: 146 - protein, 111 aa, Artificial Sequence; identical to SEQ ID NO: 138
  Description of Artificial Sequence Synthetic polypeptide

SEQ ID NO: 147 - protein, 116 aa, Artificial Sequence
  Description of Artificial Sequence Synthetic polypeptide
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTLVTVSG

SEQ ID NO: 148 - protein, 111 aa, Artificial Sequence
  Description of Artificial Sequence Synthetic polypeptide
QSVLTQPPSVSAAPGQKVTISCSGGSTNIGNSYVSWYQRLPGTAPKLLIYDNNKRPPGIPDRFSGSKSGTSATLAITGLQTGDEADYYCGTWDTSTVWEWPFGTGTKLTVL

SEQ ID NO: 149 - protein, 116 aa, Artificial Sequence
  Description of Artificial Sequence Synthetic polypeptide
QVQLVQSGAEVKKPGASVKVSCKASGYAFTSYYMHWARQAPGQGLEWVGIINPSGGSTSYAQKFQGRVTMTRDTSTSTVYMELSSLRSGDTAVYYCARGKYWMYDWGKGTLVTVSS

SEQ ID NO: 150 - protein, 111 aa, Artificial Sequence
  Description of Artificial Sequence Synthetic polypeptide
QAVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTGWEWPFGTGTKLTVL

SEQ ID NO: 151 - protein, 116 aa, Artificial Sequence
  Description of Artificial Sequence Synthetic polypeptide
QVQLVQSGAEVKKPGASVKVSCKASGYAFTSYYMHWVRQAPGQGLEWMGIINPRGGSTSYAQKFQGRVTMTRDTSTSTVYMELSSLRSEDTAVYYCARGKYWMYDWGKGTLVTVSS

SEQ ID NO: 152 - protein, 111 aa, Artificial Sequence
  Description of Artificial Sequence Synthetic polypeptide
QSVLTQPPSVSAAPGQKVTISCSGGGSSIGNSYVSWYQQLPGTAPKLLIYDNNKRPSGIPDRFSGSKSGTSATLGITGLQTGDEADYYCGTWDTSPVWEWPFGTGTKLTVL

SEQ ID NO: 153 - protein, 116 aa, Artificial Sequence
  Description of Artificial Sequence Synthetic polypeptide
EVQLLESGGGLVQPGGSLRLSCAVSGFTFSNYAMSWVRQAPGKGLEWVSAISSGGGNIYYADSVKGRFTISRDNSKNTLYLQMNSLRAEDTAVYYCAKLRRYFDYWGQGTLVTVSS

SEQ ID NO: 154 - protein, 121 aa, Artificial Sequence
  Description of Artificial Sequence Synthetic polypeptide
EVQLLESGGGLVQPGGSLRLSCAASGFTFSDYAMSWVRQAPGKGLEWVSAISSGGSSIYYADSVKGRFTISRDNSKNTLHLQMNSLRAEDTAVYYCARGPQRSATAVFDYWGQGTLVTVSS

SEQ ID NO: 155 - protein, 127 aa, Artificial Sequence
  Description of Artificial Sequence Synthetic polypeptide
EVQLLESGGGLVQPGGSLRLSCAASGFTFSNYAMSWVRQAPGKGLEWVSWISPNSGNIYYADSVKGRFTISRDNSKNTLYLQMNSLRAEDTAVYYCARRPLSAAWSHSSYYNAMDVWGQGTLVTVSS

SEQ ID NO: 156 - protein, 116 aa, Artificial Sequence
  Description of Artificial Sequence Synthetic polypeptide
EVQLLESGGGLVQPGGSLRLSCAASGFTFSGYAMSWVRQAPGKGLEWVSLISHSGSNTYYADSVKGRFTISRDNSKNTLYLQMNSLRAEDTAVYYCARPHRAFDYWGQGTLVTVSS

SEQ ID NO: 157 - protein, 116 aa, Artificial Sequence
  Description of Artificial Sequence Synthetic polypeptide
EVQLLESGGGLVQPGGSLRLSCAASGFTFSNYAMSWVRQAPGKGLEWVSGISHGSGSIYYADSVKGRFTISRDNSKNTLYLQMNSLRAEDTAVYYCARPHRAFDYWGQGTLVTVSS

SEQ ID NO: 158 - protein, 116 aa, Artificial Sequence
  Description of Artificial Sequence Synthetic polypeptide
EVQLLESGGGLVQPGGSLRLSCAASGFTFSNYAMSWVRQAPGKGLEWVSGISHGNGSIYYADSVKGRFTISRDNSKNTLYLQMNSLRAEDTAVYYCAKTGRHFDYWGQGTLVTVSS

SEQ ID NO: 159 - protein, 116 aa, Artificial Sequence
  Description of Artificial Sequence Synthetic polypeptide
EVQLLESGGGLVQPGGSLRLSCAASGFTFSNYAMSWVRQAPGKGLEWVSSISPSGSSIYYADSVKGRFTISRDNSKNTLYLQMNSLRAEDTAVYYCARSYRAFDYWGQGTLVTVSS

SEQ ID NO: 160 - protein, 116 aa, Artificial Sequence
  Description of Artificial Sequence Synthetic polypeptide
EVQLLESGGGLVQPGGSLRLSCAASGFTFSNYAMSWVRQAPGKGLEWVSAISPSGGSIYYADSVKGRFTISRDNSKNTLYLQMNSLRAEDTAVYYCARAKRAFDYWGQGTLVTVSS

SEQ ID NO: 161 - protein, 116 aa, Artificial Sequence
  Description of Artificial Sequence Synthetic polypeptide
EVQLLESGGGLVQPGGSLRLSCAASGFTFSNYAMSWVRQAPGKGLEWVSAISPGSGSTYYADSVKGRFTISRDNSKNTLYLQMNSLRAEDTAVYYCAKFRRHFDYWGQGTLVTVSS

SEQ ID NO: 162 - protein, 116 aa, Artificial Sequence
  Description of Artificial Sequence Synthetic polypeptide
EVQLLESGGGLVQPGGSLRLSCAASGFTFSNYAMSWVRQAPGKGLEWVSAISSGGGNIYYADSVKGRFTISRDNSKNTLYLQMNSLRAEDTAVYYCARVHRAFDYWGQGTLVTVSS

SEQ ID NO: 163 - protein, 116 aa, Artificial Sequence
  Description of Artificial Sequence Synthetic polypeptide
EVQLLESGGGLVQPGGSLRLSCAASGFTFSNYAMSWVRQAPGKGLEWVSAITSSGRSIYYADSVKGRFTISRDNSKNTLYLQMNSLRAEDTAVYYCARVHRAFDYWGQGTLVTVSS

SEQ ID NO: 164 - protein, 116 aa, Artificial Sequence
  Description of Artificial Sequence Synthetic polypeptide
EVQLLESGGGLVQPGGSLRLSCAASGFTFSNYAMSWVRQAPGKGLEWVSAITSSGANIYYADSVKGRFTISRDNSKNTLYLQMNSLRAEDTAVYYCARVHRAFDYWGQGTLVTVSS

SEQ ID NO: 165 - protein, 116 aa, Artificial Sequence
  Description of Artificial Sequence Synthetic polypeptide
EVQLLESGGGLVQPGGSLRLSCAASGFTFSNYAMSWVRQAPGKGLEWVSAITSSGGNIYYADSVKGRFTISRDNSKNTLYLQMNSLRAEDTAVYYCARVHRAFDYWGQGTLVTVSS

SEQ ID NO: 166 - protein, 116 aa, Artificial Sequence
  Description of Artificial Sequence Synthetic polypeptide
EVQLLESGGGLVQPGGSLRLSCAASGFTFSNYAMSWVRQAPGKGLEWVSAITAGGGSIYYADSVKGRFTISRDNSKNTLYLQMNSLRAEDTAVYYCARVHRAFDYWGQGTLVTVSS

SEQ ID NO: 167 - protein, 116 aa, Artificial Sequence
  Description of Artificial Sequence Synthetic polypeptide
EVQLLESGGGLVQPGGSLRLSCAASGFTFSRHAMAWVRQAPGKGLEWVSAITSSGRSIYYADSVKGRFTISRDNSKNTLYLQMNSLRAEDTAVYYCARVHRAFDYWGQGTLVTVSS

SEQ ID NO: 168 - protein, 110 aa, Artificial Sequence
  Description of Artificial Sequence Synthetic polypeptide
QSVLTQPPSASGTPGQRVTISCSGSSSNIGNNYVNWYQQLPGTAPKLLIYDNSHRPSGVPDRFSGSKSGTSASLAISGLRSEDEADYYCGTWDASLSAYVFGGGTKLTVL

SEQ ID NO: 169 - protein, 110 aa, Artificial Sequence
  Description of Artificial Sequence Synthetic polypeptide
QSVLTQPPSASGTPGQRVTISCSGSSSNIGNNNVSWYQQLPGTAPKLLIYANSKRPSGVPDRFSGSKSGTSASLAISGLRSEDEADYYCGSWDDSLSAYVFGGGTKLTVL

SEQ ID NO: 170 - protein, 110 aa, Artificial Sequence
  Description of Artificial Sequence Synthetic polypeptide
QSVLTQPPSAPGTPGQRVTISCTGSSSNIGSNSVNWYQQLPGTAPKLLIYDDSHRPSGVPDRFSGSKSGTSASLAISGLRSEDEADYYCDAWDSSLSAYVFGGGTKLTVL

SEQ ID NO: 171 - protein, 110 aa, Artificial Sequence
  Description of Artificial Sequence Synthetic polypeptide
QSVLTQPPSASGTPGQRVTLSCTGSSSNIGSNYVSWYQQLPGTAPKLLIYADSQRPSGVPDRFSGSKSGTSASLAISGLRSEDEADYYCGTWDDSLSGYVFGGGTKLTVL

SEQ ID NO: 172 - protein, 110 aa, Artificial Sequence
  Description of Artificial Sequence Synthetic polypeptide
QSVLTQPPSASGTPGQRVTISCSSSSSNIGSNYVSWYQQLPGTAPKLLIYSDSHRPSGVPDRFSGSKSGTSASLAISGLRSEDEADYYCGSWDYSLSAYVFGGGTKLTVL

SEQ ID NO: 173 - protein, 110 aa, Artificial Sequence
  Description of Artificial Sequence Synthetic polypeptide
QSVLTQPPSASGTPGQRVTISCTGSSSNIGNNTVSWYQQLPGTAPKLLIYDNSHRPSGVPDRFSGSKSGTSASLAISGLQSEDEADYYCGSWDYSLSAYVFGGGTKLTVL

SEQ ID NO: 174 - protein, 110 aa, Artificial Sequence
  Description of Artificial Sequence Synthetic polypeptide
QSVLTQPPSASGTPGQRVTISCTGSSSNIGNNDVNWYQQLPGTAPKLLIYYDSQRPSGVPDRFSGSKSGTSASLAISGLRSEDEADYYCATWDASLSAYVFGGGTKLTVL

SEQ ID NO: 175 - protein, 110 aa, Artificial Sequence
  Description of Artificial Sequence Synthetic polypeptide
QSVLTQPPSASGTPGQRVTISCSGSSSNIGSNAVNWYQQLPGTAPKLLIYYDNQRPSGVPDRFSGSKSGTSASLAISGLRSEDEADYYCGTWDDSLNGYVFGGGTKLTVL

SEQ ID NO: 176 - protein, 110 aa, Artificial Sequence
  Description of Artificial Sequence Synthetic polypeptide
QSVLTQPPSASGTPGQRVTISCSGSSSNIGNNAVTWYQQLPGTAPKLLIYDDSHRPSGVPDRFSGSKSGTSASLAISGLRSEDEADYYCGSWDYSLSAYVFGGGTKLTVL

SEQ ID NO: 177 - protein, 110 aa, Artificial Sequence
  Description of Artificial Sequence Synthetic polypeptide
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLSGYVLGGGTKLTVL

SEQ ID NO: 178 - protein, 110 aa, Artificial Sequence; identical to SEQ ID NO: 177
  Description of Artificial Sequence Synthetic polypeptide

SEQ ID NO: 179 - protein, 110 aa, Artificial Sequence
  Description of Artificial Sequence Synthetic polypeptide
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLRGYVLGGGTKLTVL

SEQ ID NO: 180 - protein, 110 aa, Artificial Sequence
  Description of Artificial Sequence Synthetic polypeptide
QSVLTQPPSASGTPGQRVTISCSGSSSNIGSNTFNWYQQLPGTAPKLLIYADSHRPSGVPDRFSGSKSGTSASLAISGLRSEDEADYYCGYWDYSLSGYVLGGGTKLTVL

SEQ ID NO: 181 - protein, 110 aa, Artificial Sequence; identical to SEQ ID NO: 177
  Description of Artificial Sequence Synthetic polypeptide

SEQ ID NO: 182 - protein, 111 aa, Artificial Sequence
  Description of Artificial Sequence Synthetic polypeptide
QSVLTQPPSASGTPGQRVTISCSGSSANSRTDGFNWYQQLPGTAPKLLIYADSHRPSGVPDRFSGSKSGTSASLAISGLRSEDEADYYCGTWDYSLSGYVLGGGTKLTVLG

SEQ ID NO: 183 - protein, 111 aa, Artificial Sequence
  Description of Artificial Sequence Synthetic polypeptide
QSVLTQPPSASGTPGQRVTISCSGSAQFGSRDNFNWYQQLPGTAPKLLIYADSHRPSGVPDRFSGSKSGTSASLAISGLRSEDEADYYCGTWDYSLSGYVLGGGTKLTVLG

SEQ ID NO: 184 - protein, 111 aa, Artificial Sequence
  Description of Artificial Sequence Synthetic polypeptide
QSVLTQPPSASGTPGQRVTISCSGSTKQMHNYQFNWYQQLPGTAPKLLIYADSHRPSGVPDRFSGSKSGTSASLAISGLRSEDEADYYCGTWDYSLSGYVLGGGTKLTVLG

SEQ ID NO: 185 - protein, 111 aa, Artificial Sequence
  Description of Artificial Sequence Synthetic polypeptide
QSVLTQPPSASGTPGQRVTISCSGSLLRGENLQFNWYQQLPGTAPKLLIYADSHRPSGVPDRFSGSKSGTSASLAISGLRSEDEADYYCGTWDYSLSGYVLGGGTKLTVLG

SEQ ID NO: 186 - protein, 111 aa, Artificial Sequence
  Description of Artificial Sequence Synthetic polypeptide
QSVLTQPPSASGTPGQRVTISCSGSPLFPDSGSFNWYQQLPGTAPKLLIYADSHRPSGVPDRFSGSKSGTSASLAISGLRSEDEADYYCGTWDYSLSGYVLGGGTKLTVLG

SEQ ID NO: 187 - protein, 111 aa, Artificial Sequence
  Description of Artificial Sequence Synthetic polypeptide
QSVLTQPPSASGTPGQRVTISCSGSAALDLSPSFNWYQQLPGTAPKLLIYADSHRPSGVPDRFSGSKSGTSASLAISGLRSEDEADYYCGTWDYSLSGYVLGGGTKLTVLG

SEQ ID NO: 188 - protein, 117 aa, Artificial Sequence
  Description of Artificial Sequence Synthetic polypeptide
QVQLVQSGAEVKKPGASVKVSCKASGYTFTNYGISWVRQAPGQGLEWMGWISVYNGKTNYAQKLQGRVTMTTDTSTTTAYMEMRSLRSDDTAVYYCARGSGYDLDYWGQGTLVSVSS

SEQ ID NO: 189 - protein, 128 aa, Artificial Sequence
  Description of Artificial Sequence Synthetic polypeptide
EVQLVESGGGLVQPGGSLRLSCAASGFTFSSFWMTWVRQAPGKGLEWVANIKQDGSEKYYVDSVKGRFTISRDNAKNSLYLQMNSLRAEDTAVYYCARDPGRTMVRGGIRYYYGMDVWGQGTTVTVSS

SEQ ID NO: 190 - protein, 122 aa, Artificial Sequence
  Description of Artificial Sequence Synthetic polypeptide
EVKLAESGGGLVQPGGSLRLSCAASGFTFSSHWMNWVRQAPGKGLEWVANIKQDGSDKYYVDSVKGRFTISRDNAKNSLYLQLNSLIAEDTAVYYCARDRGVRPPRGAFDIWGQGTMVTVSS

SEQ ID NO: 191 - protein, 128 aa, Artificial Sequence
  Description of Artificial Sequence Synthetic polypeptide
QVQLVQSGAEVKKPGASVKVSCKASGYTFNSYGISWVRQAPGQGLEWMGWIRTYNGNTNYAQKLQGRVTMTTDTSTSTAYMELRSLRSDDTAVYYCARDEARIVVAGTTPYYYGMDVWGQGTTVTVSS

SEQ ID NO: 192 - protein, 117 aa, Artificial Sequence
  Description of Artificial Sequence Synthetic polypeptide
QVQLVESGGGLVQPGGSLRLSCAVSGFTISDHYMSWIRQAPGKGLEWISYISSSGSKIYYADSVKGRFTISRDNAKNSLFLQMNSLRAEDTAVYYCARTRQLVGDYWGQGTLVTVSS

SEQ ID NO: 193 - protein, 124 aa, Artificial Sequence
  Description of Artificial Sequence Synthetic polypeptide
EVQLVESGGGLVQPGRSLRLSCAASGFTFDNYAMHWVRQAPGKGLEWVSGIRWNSGSIGYADSVKGRFTISRDNAKNSLYLQMNSLRAEDTALYYCAKEGGYSGYRPGPFFDYWGQGTLVTVSS

SEQ ID NO: 194 - protein, 117 aa, Artificial Sequence
  Description of Artificial Sequence Synthetic polypeptide
QVQLVQSGAEVKKPGASVKVSCKASGYTFTNYGISWVRQAPGQGLEWMGWISVYNGHTNYAQKLQGRVTMTTDTSTSTAYMELRSLRSDDTAVYYCARGSGYDFDSWGQGTLVTVSS

SEQ ID NO: 195 - protein, 116 aa, Artificial Sequence
  Description of Artificial Sequence Synthetic polypeptide
QVQLVQSGAEVKKPGASVKVSCKASRYTFTSYDINWVRQATGQGLEWMGWMNPNSGNTGYAQKFQGRVTMTRNTSTSTAYMELSSLRSEDTAVYYCARVRRFFDYWGQGTLVTVSS

SEQ ID NO: 196 - protein, 117 aa, Artificial Sequence
  Description of Artificial Sequence Synthetic polypeptide
QVQLVQSGPEVKKPGASVKVSCKASGYTFTNYGISWVRQAPGQGLEWMGWISVYNGNINYAQKLQGRVTMTTDTSTSTAYMDLRSLRSDDTAVYYCARGSGYDFDYWGQGTLVTVSS

SEQ ID NO: 197 - protein, 122 aa, Artificial Sequence
  Description of Artificial Sequence Synthetic polypeptide
QVQLVQSGAEVKKPGASVKVSCKDSAYTFNRYGISWVRQAPGQGLEWMGWISAYTGNTVYAQKLQGRVTMTTDNSTSTAYMELRSLRSDDTAVYYCARDKSIFGVVRGFDYWGQGTLVTVSS

SEQ ID NO: 198 - protein, 107 aa, Artificial Sequence
  Description of Artificial Sequence Synthetic polypeptide
AIQMTQSPSSLSASVGDRVTITCRASQGIRNALGWYQQKPGKAPKLLIYAASSLQSGVPSRFSGSGSGTDFTLTFSSLQPEDFATYYCLQDFNYPYTFGQGTKLEIK

SEQ ID NO: 199 - protein, 107 aa, Artificial Sequence
  Description of Artificial Sequence Synthetic polypeptide
DIQMTQSPSSVSASVGDRVTISCRASQGVSSWLAWYQQKPGNAPKLLISAASSIQSGVPSRFSGSGSGTDFTLTISSLQPEDFATYYCQQANSFPLTFGGGTKVEIK

SEQ ID NO: 200 - protein, 107 aa, Artificial Sequence
  Description of Artificial Sequence Synthetic polypeptide
DIQMTQSPSSVSASVGDRVTITCRASQGISSWLAWYQQKPGKAPKLLIYAASSFQSGVPSRFSGSGSGTDFTLTISSLQPEDFATYFCQQANSFPLTFGGGTTVEIK

SEQ ID NO: 201 - protein, 107 aa, Artificial Sequence
  Description of Artificial Sequence Synthetic polypeptide
DIQMTQSPSSVSASVGDRVTITCRASQDISIWLAWYQQSPGKAPKLLINVASRLQSGVPSRFSGSGSGTDFTLTINSLQPEDFVTYYCQQANSFPITFGQGTRLATK

SEQ ID NO: 202 - protein, 107 aa, Artificial Sequence
  Description of Artificial Sequence Synthetic polypeptide
DIQLTQSPSFLSASVGDRVTITCWASQGISSYLAWYQQKPGKAPKLLIFAASTLQSGVPSRFSGSGSGTEFTLTISSLQPEDFATYYCQQLNSYPLTFGGGTKVEIR

SEQ ID NO: 203 - protein, 107 aa, Artificial Sequence
  Description of Artificial Sequence Synthetic polypeptide
EIVMTQSPATLSVSPGERATLSCRASQSVNYNLAWYQHKPGQAPRLLIYGASTRATGIPARFSGSGSGTEFTLTISSLQSEDFAVYYCQQYNNWPLTFGGGTKVEIK

SEQ ID NO: 204 - protein, 107 aa, Artificial Sequence
  Description of Artificial Sequence Synthetic polypeptide
AIQMTQSSSSLSASVGDRVTITCRASQAIRNALGWYQQKPGKAPKVLIYAASSLQSGIPSRFSGSGSGTDFTLTISSLQPEDFATYYCLQDYDYPYTFGQGTKLEIK

SEQ ID NO: 205 - protein, 107 aa, Artificial Sequence
  Description of Artificial Sequence Synthetic polypeptide
DIQLTQSPSFLSASVGDRVTITCWASQGIISYLAWYQQKPGKAPKLLIYAASTLHSGVPSRFSGSGSGTEFTLTISSLQPEDFATYYCHQLKSYPITFGQGTRLEIK

SEQ ID NO: 206 - protein, 107 aa, Artificial Sequence
  Description of Artificial Sequence Synthetic polypeptide
AIQMTQSPSSLSASVGDRVTITCRASQDIRNALGWYQQKPGKAPKLLIYAASSLQSGVPSRFSGSASGTDFTLTISSLQPEDFAAYYCLQDYNYPYTFGQGTKLEIK

SEQ ID NO: 207 - protein, 107 aa, Artificial Sequence
  Description of Artificial Sequence Synthetic polypeptide
EIVMTQSPVTLSLSPGERATLPCRASQSVSSSLAWYQQKAGQSPRLLIYGASTRATGIPARFSGSGSGTEFTLTISNLQSEDFAVYYCQQYNNWPLTFGGGTKVEIK